(19) United States
(12) Patent Application Publication
(10) Pub. No.: US 2024/0424023 A1
(43) Pub. Date: Dec. 26, 2024

(54) METHOD

(71) Applicants: Pietro Genovese, Boston, MA (US); UCL BUSINESS LTD, London (GB)

(72) Inventors: Pietro Genovese, Boston, MA (US); Thomas Fox, London (GB); Emma Morris, London (GB); Claire Booth, London (GB); Ben Houghton, London (GB)

(21) Appl. No.: 18/690,333

(22) PCT Filed: Sep. 9, 2022
(86) PCT No.: PCT/GB2022/052295

§ 371 Date: Mar. 8, 2024

(30) Foreign Application Priority Data
Sep. 10, 2021 (GB) 2112922.6

(51) Int. Cl. A61K 35/17; A61K 39/00; A61K 48/00; A61P 35/00; C12N 5/0783; C12N 9/22; C12N 15/11; C12N 15/86

(52) U.S. Cl. CPC A61K 35/17; A61K 39/4611; A61K 39/464411; A61K 48/005; A61P 35/00; C12N 5/0636; C12N 9/22; C12N 15/111; C12N 15/86; C12N 2310/20; C12N 2750/14143

(57) ABSTRACT

The present invention provides a method of engineering a cell, comprising the steps of introducing into the cell: i) a site-directed nuclease which is capable of cleaving a target nucleotide sequence at the 3′-end of Intron 1 of a Cytotoxic T-Lymphocyte Associated Protein 4 gene (CTLA4); and ii) a nucleic acid construct which comprises a nucleic acid sequence comprising one or more of exon 2, exon 3 and exon 4 of CTLA4, or a sequence with at least 70% identity to exon 2, exon 3 and/or exon 4 of the CTLA4 gene, and 5′- and 3′-homology arms, wherein each of the 5′ and '3 homology arms is essentially complementary to a sequence flanking the target nucleotide sequence at the 3′-end of Intron 1 of CTLA4.

### FIELD OF THE INVENTION

The present invention relates to a method for engineering a cell in order to restore and/or modulate CTLA4 expression, site-directed nucleases targeting intron 1 of CTLA4, and nucleic acid constructs comprising CTLA4 exon sequences.

### BACKGROUND TO THE INVENTION

Cytotoxic T-Lymphocyte Associated Protein 4 (CTLA4) is a critical negative immune regulator. CTLA4 is expressed constitutively on regulatory T cells (T_{regs}) and on conventional T cells (T_{cons}) upon activation. CTLA4 competes with its opposing stimulatory receptor, CD28 for the shared ligands, CD80 and CD86, which are expressed on antigen presenting cells (APCs). CTLA4 binds its ligands and then removes them from APCs by the process of transendocytosis (TE). CTLA4 has a higher affinity for CD80/86 than CD28 and can therefore prevent T cell costimulatory signals through this pathway.

Germline heterozygous mutations in CTLA4 results in an immune dysregulation syndrome in humans arising from hyperactivation of effector T cells and dysregulation of T_{regs}. CTLA4 haploinsufficiency is characterised by hypogammaglobulinemia, recurrent infections and profound autoimmunity including cytopenias, enteropathy and lymphocytic infiltration.

CTLA4 haploinsufficiency has a heterogeneous genetic landscape with over 45 different germline heterozygous mutations which result in a clinical phenotype described. Disease-causing mutations have been described in the first three of the four exons, however the majority (>80%) are found in exons 2 and 3, the ligand binding and transmembrane domains, respectively (Schwab et al., 2018, J Allergy Clin Immunol, 142(6): 1932-1946).

The immunological phenotype amongst patients and carriers is also heterogenous, although hypogammaglobulinaemia is common. Patients and carriers have an increased percentage of CD4+FOXP3+T_{reg} cells compared to healthy controls (Schwab et al., 2018, J Allergy Clin Immunol, 142(6): 1932-1946). Within the T_{reg} fraction, CTLA4 expression is reduced in both resting and activated cells compared to healthy controls in a majority of affected individuals (Schubert et al., 2014, Nat Med, 20(12): 1410-1416).

Management of CTLA4 deficiency is challenging. The CTLA4 mimetic fusion proteins abatacept and belatacept can result in clinical improvement, however concomitant immunosuppression is often required to control the autoimmune manifestations.

Allogeneic haematopoietic stem cell transplantation (AlloHSCT) has been successfully performed in patients with CTLA4 deficiency prompted by severe, life-threatening complications such as treatment-resistant cytopenias, enteropathy, lymphoproliferation or development of malignancy or severe infections. However, alloHSCT carries a significant risk of mortality in addition to the risks of morbidity, in particular from graft-versus-host disease (GVHD). In addition, patients can deteriorate rapidly after developing a severe complication of their disease, making the window in which to transplant extremely narrow.

Autologous gene therapy would offer the benefit of a potentially curative intervention without the immunological complications of an allogeneic approach. Autologous gene therapy using viral gene-addition strategies has been shown to be effective in inborn errors of immunity (IEIs) including adenosine-deaminase (ADA) deficient severe combined immunodeficiency (SCID), X-linked SCID, chronic granulomatous disease (CGD) and Wiskott-Aldrich syndrome (WAS).

Gene therapy using retrovirus or lentivirus vectors can correct the immune defect in disorders where the protein is absent. However, these platforms are unlikely to be successful in disorders where the gene requires close regulation, or in haploinsufficiency where some normal protein is present. Without wishing to be bound by theory, in CTLA4 haploinsufficiency, gene addition by viral vector would likely result in supraphysiological expression of CTLA4 in T cells in both resting and activated states. Furthermore, gene addition by viral vector carries a risk of insertional mutagenesis.

Given the challenges associated with gene therapy in haploinsufficiency and the differential expression of CTLA4 in conventional T cells in the resting and activated state, there is a need for an improved method for a targeted gene editing approach to modulate and/or restore expression of CTLA4 in deficient cells.

### SUMMARY OF THE INVENTION

The present invention provides a method for engineering cells in order to restore and/or modulate expression of CTLA4, comprising targeted insertion of replacement CTLA4 sequences in to the 3′-end of intron 1 of the endogenous CTLA4 gene.

Without wishing to be bound by theory, it is considered that the present methods provide several advantages, including regulation of expression by the endogenous CTLA4 promoter and preservation of intronic machinery.

Furthermore, the coding sequence of endogenous CTLA4 remains intact. Gene insertion is typically facilitated by the repair of nuclease-induced DNA double-stranded breaks (DSBs) by homology-directed repair (HDR). However, this process is limited by other repair pathways, such as non-homologous end joining (NHEJ), competing with HDR. In CTLA4 haploinsufficiency, preservation of the coding sequence ensures that some level of functional CTLA4 expression is retained in cells undergoing NHEJ (therefore acquiring NHEJ-mediated indel mutations). As a result, the total level of functional protein in the cell population may be increased.

The method of the present invention may further provide a “one-size-fits-most” strategy for correcting CTLA4 deficiency. While correction of single point mutations is useful in diseases largely attributed to a single mutation, CTLA4 deficiency has a heterogeneous mutational landscape and would therefore require numerous targeted gene edits. As more than 80% of disease-causing mutations occur in exons 2 and 3 of CTLA4, the methods of the present invention are expected to benefit the majority of patients.

In addition, the ability to manipulate CTLA4 has potential applications outside of correction of CTLA4 haploinsufficiency. Given its critical role in immune regulation and constitutive expression in T regulatory cells, CTLA4 gene edited cellular therapies have application to autoimmune diseases. As understanding of the interaction of CTLA4 with its ligands CD80 and CD86 increases it is becoming apparent that certain mutations in CTLA4 can alter the function of the protein in terms of affinity and avidity to ligand, ligand binding and cellular signalling.

Thus, in a first aspect, the present invention provides a method of engineering a cell, comprising the steps of introducing into the cell:
i) a site-directed nuclease which is capable of cleaving a target nucleotide sequence at the 3′-end of Intron 1 of a Cytotoxic T-Lymphocyte Associated Protein 4 gene (CTLA4); and
ii) a nucleic acid construct which comprises a nucleic acid sequence comprising one or more of exon 2, exon 3 and exon 4 of CTLA4, or a sequence with at least 70% identity to exon 2, exon 3 and/or exon 4 of the CTLA4 gene, and 5′- and 3′-homology arms, wherein each of the 5′ and 3′ homology arms is essentially complementary to a sequence flanking the target nucleotide sequence at the 3′-end of Intron 1 of CTLA4.

In a further aspect, the present invention provides a site-directed nuclease which is capable of cleaving a target nucleotide sequence at the 3′-end of Intron 1 of CTLA4.

The target nucleotide sequence may be within the sequence which corresponds to position 1268-2534, 1550-2400, 1900-2330 or 2097-2116 of SEQ ID No: 2.

Suitably the target nucleotide sequence is within the sequence which corresponds to position 1900-2330 of SEQ ID No: 2.

Suitably the target nucleotide sequence is within the sequence which corresponds to position 2097-2116 of SEQ ID No: 2.

The nuclease may be a CRISPR-associated protein (Cas) in complex with a gRNA molecule, wherein the gRNA is complementary to the target nucleotide sequence at the 3′-end of Intron 1 of CTLA4.

The CRISPR-associated protein may be Cas9.

In another aspect, the present invention provides a gRNA comprising any of SEQ ID Nos: 11, 14, or 17, preferably SEQ ID No: 11.

In another aspect, the present invention provides a vector comprising the gRNA according to the invention.

In a further aspect, the present invention provides a nucleic acid construct which comprises a nucleic acid sequence comprising one or more of exon 2, exon 3 and exon 4 of the CTLA4 gene, or a sequence with at least 70% identity to exon 2, exon 3 and/or exon 4 of the CTLA4 gene, and 5′- and 3′-homology arms, wherein each of the 5′ and 3′ homology arms is essentially complementary to a sequence flanking a target nucleotide sequence at the 3′-end of Intron 1 of CTLA4

The nucleic acid construct may further comprise a reporter gene.

In a further aspect, the present invention provides a vector comprising the nucleic acid construct according to the invention.

In another aspect, the present invention provides a kit of polynucleotides comprising:
i) a polynucleotide comprising the gRNA according to the invention;
ii) a polynucleotide comprising the nucleic acid construct according to the invention; and
iii) optionally, a polynucleotide encoding a Cas protein.

In another aspect, the present invention provides a kit of vectors comprising:
i) a vector according to the invention;
ii) a vector according to the invention.

In a further aspect, the present invention provides a cell comprising the nucleic acid construct according to the invention.

In another aspect, the present invention provides a cell engineered according to the method of the invention.

The cell may be a haematopoietic stem cell (HSC).

The cell may be a CD3+ T cell, preferably a regulatory T cell.

The cell may be isolated from a subject.

The cell may comprise a mutation in endogenous CTLA4 and/or be deficient in endogenous CTLA4 expression.

In another aspect, the present invention provides a pharmaceutical composition comprising the cell according to the invention.

In a further aspect, the present invention provides a method of treating and/or preventing a disease in a subject comprising administering the cell according to invention.

The method may comprise the following steps:
i) isolation of a cell-containing sample from a subject;
ii) introducing into the cells a nucleic acid construct according to the invention or a vector according to the invention and a site-directed nuclease according to the second aspect of the invention; and
iii) administering the cells from (ii) to a subject.

In a further aspect, the present invention provides use of a cell according to the invention in the manufacture of a medicament for the treatment and/or prevention of a disease.

In another aspect, the present invention provides a cell according to the invention, for use in treating and/or preventing disease in a subject.

The disease may be an immune deficiency disease, an autoimmune disease, a cancer or associated with solid organ and/or haematopoietic stem cell transplantation.

### DESCRIPTION OF THE FIGURES

FIG. 1—(A) Schematic representation of the editing strategy targeting the first exon of CTLA4 with a CTLA4 cDNA-P2A-GFP-WPRE sequence in an AAV6 HDR donor flanked by two asymmetrical homology arms. (B) FACS plots of the editing strategy shown in (A) demonstrating a non-edited control (left), gRNA only control with resulting knock down of CTLA4 (centre) and HDR mediated by the CTLA4 cDNA-P2A-GFP-WPRE AAV6 donor (48.8% GFP positive cells). (C) Schematic representation of the two intronic editing strategies with the difference being the substitution of the WPRE sequence for the CTLA4 3′UTR sequence downstream of the transgene and GFP reporter. (D) Graph showing % CD4+ cells expressing surface CTLA4 in wild type unedited cells (mean 51.83%, SD 14.52), cells edited with the exon 1 approach (mean 7.2%, SD 4.3), and cells edited with the intronic approach (mean 58.67%, SD 5.13), n=3, demonstrating that the intronic approach results is less disruption of the CTLA4 ORF and comparable protein expression compared to an unedited CD4+ T cell. (E) ICE analysis showing the spectrum of indels created by the intronic gRNA with no change in protein expression by FACS (shown in F). (F) FACS plots showing CTLA4 expression and GFP expression (HDR) in cells edited with the intronic gRNA alone (centre left), the P2A-GFP-WPRE AAV6 donor (centre right) and the P2A-GFP-3′UTR donor (far right). (G) Average HDR rate (n=3, percentage GFP+ in cells from separate healthy donors). Exon 1 approach mean=42.47% GFP+, SD 8.12, intronic WPRE donor mean=64.63% GFP+, SD 3.06, Intron 3′UTR donor mean=38.13% GFP+, SD 2.70. (H) Schematic representation of the lentivirus vector incorporating the CTLA4 cDNA, P2A sequence followed by a GFP reporter. Below is a representative FACS plot demonstrating transduction of 87.9% of healthy donor CD4+ T cells.

FIG. 2—(A) Schematic representation of transendocytosis assay. Cells (edited or unedited controls) are incubated in a 5:1 ratio with DG75 cells expressing either fluorescent labelled (mCherry) CD80 or CD86. Uptake of ligand can then be assessed by flow cytometry (mCherry uptake into T cells). (B) FACS plots gated on lymphocytes>singlets>live cells>CD3+>CD4+ and GFP+ cells in the edited conditions, demonstrating transendocytosis of CD80 and CD86 in cells from healthy individuals subjected to different conditions. The top row is unedited healthy CD4+ cells. Normal uptake of CD80 and CD86 is observed as mCherry/CTLA4 positivity in the upper right quadrants. When a gRNA is used in the absence of a repair template, resulting knock out of CTLA4, transendocytosis of CD80 and CD86 is abolished (Second row). Using the exon 1 editing approach (third row) transendocytosis is restored but to a lower level than the unedited control. When the intronic editing approach is used (bottom row) transendocytosis of CD80 and CD86 is comparable to wild-type cells. (C) mCherry uptake relative to the unedited control with the different conditions. Transduction of healthy CD4+ T cells with the lentiviral vector results in supraphysiological TE of CD80 and CD86. (D) Kinetics of surface CTLA4 expression in healthy CD4+ T cells following transduction with the lentiviral vector or editing with the intronic approach (WPRE-containing donor).

FIG. 3—(A) HDR rates (% cells GFP+) in edited CD4+ cells from patients with CTLA4 haploinsufficiency resulting from three different mutations. (B) Overnight TE assay gated on CD3+ CD4+ FOXP3+ cells in healthy control (top row), c. 193_203delAAAGCCACTGA heterozygote mutant cells (middle row) and c.193_203delAAAGCCACTGA corrected with the intron editing strategy (bottom row). (C) Overnight TE assay gated on CD3+ CD4+ FOXP3+ cells in healthy control (top row), c.371A>C heterozygote mutant cells (middle row) and c.371A>C corrected with the intronic editing strategy (bottom row). (D) Overnight TE assay gated on CD3+ CD4+ FOXP3+ cells in healthy control (top row), c.223C>T heterozygote mutant cells (middle row), and c.223C>T heterozygote mutant cells corrected with the intronic editing strategy (bottom row). (E) Graph showing the increase in ligand acquisition (% CD4+ FOXP3+ T cells mCherry positive in patient cells after editing relative to healthy control. (F) Graph showing restoration of total CTLA4 in hetoerozygote mutant T_{regs} following editing. Note that the c.223C>T had increased total CTLA4 compared to healthy control and this was unchanged after editing.

FIG. 4—(A) Schematic of experimental protocol for expansion and editing of T_{regs}. (B) HDR rates in healthy donor T_{regs} across three separate experiments from three healthy donors. (C) Confocal microscopy images of edited T_{reg} (green and centre) following overnight TE. Colocalisation of CTLA4 with its ligands CD80 and CD86 is observed.

FIG. 5—Assessment of T cell GT for CTLA4 insufficiency using an in vivo murine model: (A) FACS plots demonstrating typical editing efficiencies achieved in murine CTLA4−/− T cells (GFP+ cells, upper plots) with restoration of CTLA4 expression in both the FOXP3+ and FOXP3− compartments (lower plots). (B) FACS plots post sort demonstrating % GFP+ in the sorted edited cells (upper plots) and CTLA4 expression in FOXP3− and FOXP3+ cells in the two sorted populations (GFP− left lower plot, GFP+ left upper plot). (C) Serial tail vein bleeds demonstrating persistence and stability of the GFP+ population after adoptive transfer. (D) Lymph node and spleen size in mice that received wild type T cells (left) mock edited and GFP− cells (middle) and GFP+ enriched edited cells (right). (E) Lymph node (left) and spleen (right) cell counts in mice that received wild-type T cells, mock edited, GFP− and GFP+ T cells. (F) Number of Tconv per μg of heart tissue in mice that received WT, mock edited, GFP− and GFP+ T cells. (G) Representative FACS plots (left) and collated data (right) showing CTLA4 expression in lymph node Treg and Tconv cells. (H) Representative FACS plots (left) and collated data (right) showing CTLA4 expression in spleen Treg and Tconv cells. Data collated from 2 independent experiments; n=4-5. One-way ANOVA; mean±SD are shown; ****, p<0.0001; ***, p<0.001; **, p<0.01; *, p<0.05; ns, not significant.

FIG. 6—(A) Graphs showing increase in CTLA4 and FOXP3 MFI in unedited cells and edited cells when costimulated with CD80 and CD86. (B) Frequency of FOXP3+ amongst dividing unedited and edited T cells following incubation with either CD80 or CD86. CD86-CD28 interactions drive accumulation or Treg.

### DETAILED DESCRIPTION

### Method of Engineering a Cell

The present invention provides a method of engineering a cell, comprising the steps of introducing into the cell:
i) a site-directed nuclease which is capable of cleaving a target nucleotide sequence at the 3′-end of Intron 1 of a Cytotoxic T-Lymphocyte Associated Protein 4 gene (CTLA4); and
ii) a nucleic acid construct which comprises a nucleic acid sequence comprising one or more of exon 2, exon 3 and exon 4 of CTLA4, or a sequence with at least 70% identity to exon 2, exon 3 and/or exon 4 of the CTLA4 gene, and 5′- and 3′-homology arms, wherein each of the 5′ and 3′ homology arms is essentially complementary to a sequence flanking the target nucleotide sequence at the 3′-end of Intron 1 of CTLA4.

The site-directed nuclease and nucleic acid construct may be introduced into the cell by any suitable means. For example, by transfecting the cell with DNA or RNA coding for the site-directed nuclease and/or nucleic acid construct, or transfection with a viral vector.

Alternatively, the nuclease may be introduced into the cell as a protein. Methods for introducing proteins into cells include, but are not limited to, physical methods such as microinjection and electroporation.

The site-directed nuclease used in the methods of the present invention may create a targeted DNA double stranded break (DSB).

There are two major pathways that repair DSB: non-homologous end joining (NHEJ) and homology directed repair (HDR). NHEJ uses a variety of enzymes to directly join the DNA ends, while HDR uses a homologous sequence as a template for regeneration of missing DNA sequences at the break point.

Thus, in the methods of the present invention, the site-directed nuclease creates a targeted DSB within the 3′-end of intron 1 of CTLA4, and the nucleic acid construct comprising replacement CTLA4 sequences, flanked by 5′ and 3′ homology arms, provides a repair template which enables incorporation of the replacement CTLA4 sequences into the 3′-end of intron 1 of the CTLA4 gene by homology directed repair at the site of the DSB.

Accordingly, the endogenous CTLA4 promoter, exon 1 sequence, and intronic machinery of intron 1 is preserved.

The method may comprise culturing the cells under conditions which enable incorporation of the replacement CTLA4 sequences into the 3′-end of intron 1 of the CTLA4 gene by homology directed repair at the site of the DSB.

The method may comprise the step of selecting cells in which productive incorporation of the replacement CTLA4 sequences into the 3′-end of intron 1 of the CTLA4 gene by homology directed repair at the site of the DSB has occurred. Methods for selecting cells in which productive incorporation of the replacement CTLA4 sequences has occurred are described herein.

### Site Directed Nuclease

The present invention also provides a site-directed nuclease which is capable of cleaving a target nucleotide sequence at the 3′-end of Intron 1 of a CTLA4 gene.

The site directed nuclease may be used in the methods of the present invention.

A nuclease is an enzyme that is capable of cleaving the phosphodiester bonds between nucleotides of nucleic acids. Nucleases may create single stranded breaks (nick) or double stranded breaks in their target molecules.

The term “site-directed nuclease” is synonymous with “site-specific nuclease”.

A site-directed nuclease cleaves the DNA at a predetermined location by way of a DNA binding system.

Examples of site-directed nucleases include Zinc Finger Nucleases (ZFN), TAL Effector Nucleases (TALENs), clustered regularly interspaced short palindromic repeats-associated protein (CRISPR/Cas), and Meganucleases.

Transcription activator-like effector nucleases (TALEN) are restriction enzymes that can be engineered to cut specific sequences of DNA. They are made by fusing a TAL effector (TALE) DNA-binding domain to a DNA cleavage domain.

Zinc-finger nucleases (ZFNs) are restriction enzymes generated by fusing a zinc finger DNA-binding domain to a DNA-cleavage domain. A zinc finger DNA binding domain (or binding protein) is a protein that binds DNA in a sequence-specific manner through one or more zinc fingers (regions of amino acid sequence within the binding domain whose structure is stabilized through coordination of a zinc ion). Zinc finger domains can be engineered to target specific desired DNA sequences.

Typically, the non-specific cleavage domain from the Fokl restriction endonuclease is used as the cleavage domain in ZFNs and TALENs. This cleavage domain must dimerize in order to cleave DNA therefore two ZFNs/TALENs must be designed for each target site. The two individual ZFNs/TALENs must bind opposite strands of DNA a certain distance apart.

ZFNs and TALENs make use of protein-DNA interactions for targeting specific sequences, and thus a new nuclease pair must be designed and generated for every genomic target.

In contrast, the bacterial CRISPR-Cas system uses RNA to target a nuclease to a desired DNA sequence, by means of Watson-Crick base pairing between an engineered RNA and the target DNA site.

Two components form the core of a CRISPR nuclease system, a Cas nuclease and a guide RNA (gRNA).

In one embodiment, the site-directed nuclease of the present invention comprises a Cas protein and a gRNA molecule, wherein the gRNA is complementary to a nucleotide sequence at the 3′-end of Intron 1 of CTLA4.

In one embodiment, the site-directed nuclease of the present invention is a Cas protein in complex with a gRNA molecule, wherein the gRNA is complementary to a nucleotide sequence at the 3′-end of Intron 1 of CTLA4.

The Cas may be a Class 2 Cas enzyme, for example, Cas9 (type II) or Cas12a (type V). The Cas enzyme may be CasX. Preferably, the Cas enzyme is a Cas9 enzyme.

The Cas9 enzyme may be a S. pyogenes, S. thermophiles, or S. pneumoniae Cas9, and may include mutated Cas9 derived from these organisms. The enzyme may be a Cas9 homolog or ortholog. The Cas enzyme may be codon-optimized for expression in a eukaryotic cell.

### Guide RNA (gRNA)

A guide RNA (gRNA) or single guide RNA (sgRNA) is a specific RNA sequence that recognises the target DNA region of interest and directs the Cas nuclease there for editing. The gRNA may comprise, or is made up of two parts,: crispr RNA (crRNA), a 17-20 nucleotide sequence complementary to the target DNA, and a tracr RNA, which serves as a binding scaffold for the Cas nuclease. The genomic target of the Cas enzyme may be altered by changing the crRNA sequence present in the gRNA.

It is established in the art that a Cas nuclease binds to its target sequence only in the presence of a protospacer adjacent motif (PAM). Thus, the locations in the genome that can be targeted by different Cas proteins are limited by the locations of PAM sequences. The Cas nuclease cleaves 3-4 nucleotides upstream of the PAM sequence.

For example, Cas9 may cleave upstream of the PAM sequence 5′-NGG-3′, where N is any nucleotide base.

CRISPR systems from other species of bacteria, which recognize alternative PAM sequences and utilize different crRNA and tracrRNA sequences could also be used in the methods of the invention.

Suitable gRNA target sequences can be identified using software (see for example, the Benchling online tool: https://www.benchling.com/crispr/). gRNAs can be analysed in silico for their predicted off-target effects. For example, although the specificity of Cas nucleases is tightly controlled by the target sequence of the gRNA and the presence of a PAM adjacent to the target sequence in the genome, potential off-target cleavage activity could occur on a DNA sequence with base pair mismatches. Three to five mismatches in a ˜20 nucleotide target sequence may be sufficient to cause off-target activity, with mismatches at the 5′-end of the gRNAs better tolerated than those at the 3′-end. Whilst such software may aid in the initial design and selection of candidate gRNAs, the efficiency and off-target activity of candidate gRNAs requires further in vitro validation.

The present invention provides a gRNA that is complementary to a nucleotide sequence at the 3′-end of Intron 1 of CTLA4.

In one embodiment, the gRNA comprises any of the sequences AGCUCCGGAACUAUAAUGAG (SEQ ID No: 11), GAGAAAUAGAUUCUUCAAGA (SEQ ID No: 14) or GAUAUGACAAACAGAAGACC (SEQ ID No: 17).

Preferably, the gRNA comprises the sequence SEQ ID No: 11.

A preferred gRNA of the present invention (SEQ ID NO: 11) has surprisingly high efficiency. For example, the gRNA of the present invention produces indels in over 85% of cells, as assessed by ICE (inference of CRISPR edits).

A preferred gRNA of the present invention (SEQ ID NO: 11) has surprisingly low off-target activity. For example, the gRNA of the present invention has no off-target activity, as assessed by guide SEQ analysis (an unbiased in vitro assay that assesses off-target dsDNA breaks across the whole genome).

The gRNA of the present invention may be used in combination with a Cas9 enzyme.

### Cytotoxic T-Lymphocyte-Associated Protein 4

CTLA4 also known as CD152 (cluster of differentiation 152), is a protein receptor that functions as an immune checkpoint and downregulates immune responses. CTLA4 is constitutively expressed in regulatory T cells but only upregulated in conventional T cells after activation.

CTLA4 is encoded by the CTLA4 gene.

CTLA4 comprises four exons. Exon 1 contains the leader peptide sequence and exon 2 the ligand binding site, which are both parts of the extracellular region of CTLA4. Exon 3 encodes the transmembrane region, and exon 4 the cytoplasmic tail.

An illustrative example of the human CTLA4 gene is NCBI RefSeq: NC_000002.12 (SEQ ID No: 1).

An illustrative example of the mouse CTLA4 gene is NCBI RefSeq: NC_000067.7 (SEQ ID No: 6).

Human CTLA4
(SEQ ID No: 1)
GCTTTCTATTCAAGTGCCTTCTGTGTGTGCACATGTGTAATACATATCTGGGATCAAAGCTATCTATATAAAGTC
CTTGATTCTGTGTGGGTTCAAACACATTTCAAAGCTTCAGGATCCTGAAAGGTTTTGCTCTACTTCCTGAAGACC
TGAACACCGCTCCCATAAAGCCATGGCTTGCCTTGGATTTCAGCGGCACAAGGCTCAGCTGAACCTGGCTACCAG
GACCTGGCCCTGCACTCTCCTGTTTTTTCTTCTCTTCATCCCTGTCTTCTGCAAAGGTGAGTGAGACTTTTGGAG
CATGAAGATGGAGGAGGTGTTTCTCCTACCTGGGTTTCATTTGTTTCAGCAGTCAAAGGCAGTGATTTATAGCAA
AGCCAGAAGTTAAAGGTAAAACTCCAATCTGGCTTGGCTGGCTCTGTATTCCAGGGCCAGCAGGGAGCAGTTGGG
CGGCAGCAAATAAGGCAAAGAGATAGCTCAGAACAGAGCGCCAGGTATTTAGTAGGGGCTTCATGAATGCATGTG
AGTTGGTTTAGTAGAGAGACACAGGCAATTTCAGACCCTTCTATGAGACTGGAAGTGATTTAAGAGGGAAAGGAT
AGCCATAGTCCTGAATACATTTGAGCTGGGTTTCAGGATGAGCTCACAAGTTCCTTTAAAAAAAATTGACTTAAG
CAAATCCTGGGAAGAGTTTTTTTGCTATACAATTCAAGGTTTTAAGGTCCTCGGATTCATATACTTTATAAATGA
ATTAGCCAGCTTGTTTAAAATGTAGGGAAATTGTGGGAAGAATGCCTTCTTTACTTAATTCAAGGTTTTAAGGTT
CTCTTAATCAATTCTACTAGCTAATTAGCCAATTATTTAAAAATAAAAGTTTGAAATTGCCAAAAAAAAAAGACA
AGGAAAAGGAAAGAAAGAAAGCCACCAGTCTGTTTGGCATACAATACTTAATTGTTGCCTGACCTACGTGTGGGT
TTCAGATGCAGATCCTCAGTTTTCAGCTCTTCAGAGACTGACACCAGGTTTGTTACACGGCTTAAAATGATGAGT
ATATCCATTGAATCTCAACCTTATCTCTCTCTAGACCTTCTTGGTTAAGAAACCATGTAGTTTGTATGAAGTAGG
TACTCAAAAGATATTTGATGATTTAATTTTTACTGGAGAAGAAATATTCATATATGTTTTCTTATTTTTACATGT
TTTAAATATGTAAAGATTAAATAAACACTCTTAGAAGTATTTAAATTTCCTAAAGTAAATTTATCTCAACCAGTA
ACAGGACCCTCCCAATACTGGAAAGTTGAGTGTGACCGCATTTAGTGGTGATGAGTGTGAGCTTGCTTGGGGAGA
GGGCAGGACATTTAGGATTTCTTAAGCTTAGAGTCAATACAATAAAGATTATTGAGTGCTCACTTGGGTGGGCTA
TAATCACTGCTCACAGGAGTTCATGAACCACAAGTAAAAGAGTGAGGAGATATGATTAGCTCACAAATAACTTTA
ATACAGAGCAGAAAGTAATGAACTACTGCAATGGAGTTATCACAGTGCTAAGGATGCTCAGAGGGCATCTCTGAT
AGGCAGAGGTGAGGGTTAGGGAAGGAAGCTGTAGTCTAGCTAGCTAGAGCTGCTGGAATAGACATGACAATGGCT
GCTGCCAAACTGTTTTCTCTTCTGAGGACAGATGTCCCGTGCAAGTGGCTTGGTGGAAGGGACTAGTGTCTCTAA
TATAGGGTGATTTATAAGCAGGAAAGTGTGTCCTAGAAATTCAGACCAGAGTGATAGATTGGAATTGGATCATGG
GGGACTCATTGAATGTTATTTATTGTATTTGTTTTTGCGATCAGTGTTAGTAAAGTGTCAAAGGGATTGAGCAGA
TGAGTGACATCATGCAACACAAGTTTTGAGTTTCACTTGTCAGACTGACTGGAGAGGGGCCTGGTTAGTTACAGG
AAGGTAATTTGGCATGCAGCCACTATTTTTGAGTTGATGCAAGCCTCTCTGTATGGAGAGCTGGTCTCCTTTATC
CTGTGGGAAAAGAGAACAAAGGAGCATGGGAGTGTTCAAGGGAAGGAGAAATAAAGGGCAGAGAGGCAGCGGTGG
TGTCAGGGGAAGCCCACAGGAGTTAACAGCAGGGTTGCCTCAACCTAGAGAGGAAGCGACCTGGTGCCCTCGGCT
CTGTGGCTTCCTTCATCTAACAACATCTTCCACTCTACAACAATGCCAGGGAAGGCGGAGGCTGGTACAGTGCAT
CAAGACACAGCTACTCCTGGGTGACAGAGGTTCAGGGCCAGCTCACTAAGTAGGCAGAAGTTTTTGACATATACT
TTGAGAGATAAAGCAAGATTCTGTACCTCAACCTTCAGAATTTCCCCTACCACTCATTATAGTTCCGGAGCTATA
TAGCTCCTATCATTCTATCATAACCTTAGAATACCAGAGAACATATCATCTCATCTAATTATCTCTTACTATATG
TGAAAAAAATGAAGGACATGGGGGAAGTGTGACTTGCCCCAAATCACATATTTCATGGTAGAGCCAGGTCTTCTG
TTTGTCATATCAGTGTTCTTCCTGCCACAACCATCTTGAAGAATCTATTTCTCAGTAAGAAAATATCTTTATGGA
GAGTAGCTGGAAAACAGTTGAGAGATGGAGGGGAGGCTGGGGGTGTGGAGAGGGGAAGGGGTAAGTGATAGATTC
GTTGAAGGGGGGAGAAAAGGCCGTGGGGATGAAGCTAGAAGGCAGAAGGGCTTGCCTGGGCTTGGCCATGAAGGA
GCATGAGTTCACTGAGTTCCCTTTGGCTTTTCCATGCTAGCAATGCACGTGGCCCAGCCTGCTGTGGTACTGGCC
AGCAGCCGAGGCATCGCCAGCTTTGTGTGTGAGTATGCATCTCCAGGCAAAGCCACTGAGGTCCGGGTGACAGTG
CTTCGGCAGGCTGACAGCCAGGTGACTGAAGTCTGTGCGGCAACCTACATGATGGGGAATGAGTTGACCTTCCTA
GATGATTCCATCTGCACGGGCACCTCCAGTGGAAATCAAGTGAACCTCACTATCCAAGGACTGAGGGCCATGGAC
ACGGGACTCTACATCTGCAAGGTGGAGCTCATGTACCCACCGCCATACTACCTGGGCATAGGCAACGGAACCCAG
ATTTATGTAATTGGTGAGCAAAGCCATTTCACTGAGTTGACACCTGTTGCATTGCAGTCTTCTATGCACAAAAAC
AGTTTTGTTCCTTAATTTCAGGAGGTTTACTTTTAGGACTGTGGACATTCTCTTTAAGAGTTCTGTACCACATGG
TAGCCTTGCTTATTGTGGGTGGCAACCTTAATAGCATTCTGACTGTAAAATAAAATGATTTGGGGAAGTTGGGGC
TCTCGCTCTGGAGTGCTAACCATCATGACGTTTGATCTGTACTTTTGATATGATATGATGCTCCTGGGGAAGTAG
TCCCAAATAGCCAAACCTATTGGTGGGCTACCCATGCAATTTAGGGGTGGACCTCAAGGCCTGGAAGCTCTAATG
TCCTTTTTTCACCAATGTTGGGGAGTAGAGCCCTAGAGTTTAAAACTGTCTCAGGGAGGCTCTGCTTTGTTTTCT
GTTGCAGATCCAGAACCGTGCCCAGATTCTGACTTCCTCCTCTGGATCCTTGCAGCAGTTAGTTCGGGGTTGTTT
TTTTATAGCTTTCTCCTCACAGCTGTTTCTTTGAGCAAAATGGTGAGTGTGGTGCTGATGGTGCACCATGTCTGA
TGGGGATACCTTTAGTGGTATCAACTGGCCAAAAGATGATGTTGAGTTTAGTGTTCTTGAGATGAGATGAGGCAA
TAAATGAAGAGGAAGGACAGTGGTAAAGAACGCACTAGAACCGTAGGCATTGGCATTTGAGGTTTCAGAATGACT
AATATTTTAGATGAATTTGTTTGACATTGAATGTTCATGTGCTTCTGAGCAGGGTTTCAATTTGAGTAACCGTTG
CAATAACATGGGGCAGCTGTTTTGCTCTTTGTCTTCATGACAACTGTACTTAAGCTAACAGCCCTGAAACATGAG
ATTAGGCTGGGCAGAATGCTGCTAGAGAGGACCACTTGGATGGTCTTTATTCTCCTTCTCCATGTCCCTCTCCAT
CACCTGGAAGTCACCTCTGGGTGCCACTCTGGTGCCTTCCTTGTCGAAGCTGTAGCTGCTCACATGACACCTATC
CCTGTTATCCAGTTTGCTTGACTGGGACGTTTTGCCTTCCCCTTCAGCCAGGAAGTGAAAGTCCCAGTTTTTATT
TATCACAGGTGTTGGTATTGGTGGTAGAAGAGGTAGAATTATGGAATCAGGCCTCCTGTCAGGATTTCTTTTTGA
CAGTCCCTCTCAGACACCTCTGCCTAAGGCCAGCTTTGCCATTACAAACTCTCCCTTCTCCCTCTCTCCCTTCTT
CTCTTCCTCTTCCTTCTTCTCGCTCTTTCTCTCTCTCTCTTTCTCCCTCTCTGTCTCTTATACACATACACAAAG
ATATACTCTATTCCAACATCCTCTACCCAACCTGACAGAGATGTCCTTTGCTGTAGGTTCAGCAGTGGGGATGAG
AAATACAGCTCTCAAACAGGATAACTAAAGCTTATTATCTTATCAAGCTTGTTCCCTTGCAGACAAGATTGATCA
ATTATCATAGGCTTTCTGGGTGTTCTTTCTGAAGCTTTCTCAAAGTCTCTTTCTCCTATCTTCCATTCAAGGCAA
ATGATTGCCATTTAACATCAAAATCACAGTTATTTATCTAAAATAAATTTTAATAGCTGAATCAAGAAAATCTCC
TGAGGTTTATAATTCTGTATGCTGTGAACATTCATTTTTAACCAGCTAGGGACCCAATATGTGTTGAGTTCTATT
ATGGTTAGAAGTGGCTTCCGTATTCCTCAGTAGTAATTACTGTTTCTTTTTGTGTTTGACAGCTAAAGAAAAGAA
GCCCTCTTACAACAGGGGTCTATGTGAAAATGCCCCCAACAGAGCCAGAATGTGAAAAGCAATTTCAGCCTTATT
TTATTCCCATCAATTGAGAAACCATTATGAAGAAGAGAGTCCATATTTCAATTTCCAAGAGCTGAGGCAATTCTA
ACTTTTTTGCTATCCAGCTATTTTTATTTGTTTGTGCATTTGGGGGGAATTCATCTCTCTTTAATATAAAGTTGG
ATGCGGAACCCAAATTACGTGTACTACAATTTAAAGCAAAGGAGTAGAAAGACAGAGCTGGGATGTTTCTGTCAC
ATCAGCTCCACTTTCAGTGAAAGCATCACTTGGGATTAATATGGGGATGCAGCATTATGATGTGGGTCAAGGAAT
TAAGTTAGGGAATGGCACAGCCCAAAGAAGGAAAAGGCAGGGAGCGAGGGAGAAGACTATATTGTACACACCTTA
TATTTACGTATGAGACGTTTATAGCCGAAATGATCTTTTCAAGTTAAATTTTATGCCTTTTATTTCTTAAACAAA
TGTATGATTACATCAAGGCTTCAAAAATACTCACATGGCTATGTTTTAGCCAGTGATGCTAAAGGTTGTATTGCA
TATATACATATATATATATATATATATATATATATATATATATATATATATATATATATATATTTTAATTTGATA
GTATTGTGCATAGAGCCACGTATGTTTTTGTGTATTTGTTAATGGTTTGAATATAAACACTATATGGCAGTGTCT
TTCCACCTTGGGTCCCAGGGAAGTTTTGTGGAGGAGCTCAGGACACTAATACACCAGGTAGAACACAAGGTCATT
TGCTAACTAGCTTGGAAACTGGATGAGGTCATAGCAGTGCTTGATTGCGTGGAATTGTGCTGAGTTGGTGTTGAC
ATGTGCTTTGGGGCTTTTACACCAGTTCCTTTCAATGGTTTGCAAGGAAGCCACAGCTGGTGGTATCTGAGTTGA
CTTGACAGAACACTGTCTTGAAGACAATGGCTTACTCCAGGAGACCCACAGGTATGACCTTCTAGGAAGCTCCAG
TTCGATGGGCCCAATTCTTACAAACATGTGGTTAATGCCATGGACAGAAGAAGGCAGCAGGTGGCAGAATGGGGT
GCATGAAGGTTTCTGAAAATTAACACTGCTTGTGTTTTTAACTCAATATTTTCCATGAAAATGCAACAACATGTA
TAATATTTTTAATTAAATAAAAATCTGTGGTGGTCGTTTTCCGGA
Mouse CTLA4
(SEQ ID No: 6)
CTACACATATGTAGCACGTACCTTGGATCAAAGCTGTCTATATAAAGTCCCCGAGTCTGTGTGGGTTCAAACACA
TCTCAAGGCTTCTGGATCCTGTTGGGTTTTACTCTGCTCCCTGAGGACCTCAGCACATTTGCCCCCCAGCCATGG
CTTGTCTTGGACTCCGGAGGTACAAAGCTCAACTGCAGCTGCCTTCTAGGACTTGGCCTTTTGTAGCCCTGCTCA
CTCTTCTTTTCATCCCAGTCTTCTCTGAAGGTGAGTGGAGACTTCTGGAACATGGAGGTGGAGGAGGTGTTTTTC
CTACATGGGTTTCGTTTTTTTTTTTTCTTTCAGAAGCTGAGGACAGTGATTTATAGAAATGCCAGAAGTTAAAGC
CAAACCCTCTACTTTTTGGTTTAACAGCTTCCTACAGACAAGTTCCAAGAAGAATATAGGAGGTCTAGCAGTAGC
GAAGGAGCTGAGACTAGCCACAGCAGCTTGCTGGGATCTCAGCTGGGACTTAAGGAACCATGAGAGTTCCTTAAG
TCCCATATGGGGGAGAACAAGTTCTTTGTCACTGCTAGAAAATCCTGTAAAGTTAAGAGATTTAAGGGGGGCATT
TGAAATCGTGAGTACATTTGATCTTGATATCACAGTAACATTAAGGTTTTTCTCCTTACCCCCAATCCCACCCCC
AAAACTTACCTAAATTCAAATAAATAAGAAGTTTTCCTCATTCACTTTAAGATTTTGAGACACTCTTAGTTCTTT
AAATTATCACACCTAAGGCTTAGGGGACATAGCAGAAGAGGGGGCAGAAAGACTATGAGAGCCAAAGGTACATAG
AGTTTACTGAGAGATTGTGTCTCTTAGGATGTCAAATGGTACACCCATAAAGTCTCACCAACACGACTGCCTAAA
CTTGAGCTGAATAAGGACATGAATAACAATAGACATACCCAAGTGGATGGGGAAAGACCAAGATGCTTCAGCACT
ATAGCAATAACTACAGGAAGCCAAGGAATATGGAGAGTAGGAAATTGCCAAGGGAAAAGCACACCAGCTGACTAT
CCAATAACATATGGTCAGCCTTCAAAACAAAAAGCAAATAACATATAGAATGGGCAGGTTTCATTAAGAATGACT
ATAACTATAGTCTGTGCATATCACAACAAATAATGAAAAGGGGGGCCATGAATTTGAAAGAAAAGCAAGGAGGGG
TACATGAGAAGGTTTGGAGGAGAGAAAGGAGAAATAATGTAATTATATTAATCTCAAAGAATAGAAGAAGTAGTA
AGTAAAGGGAAAATAAATTAGTTAATTATTTTAAAGATGGAATAGTTTTAAGTCGAAGGAAGAAAGAAAGGCAAA
GTGATAGAAAGACAAGAAAAGGGGAGGAAGGAAGAGAGAAGAGAGGAAGGAAGAAAGGAAGGAAGGAAGGAAGGG
AGGAAGGGAGGGAAAAAGAAAGAAGAGAGAAGGGAGGAGGACAGGAAGGAGGGAGGGAGGGAGGGAGGAAGGGAG
AAAGGGAGAAAGGAAGAAAACAATTATCAGCTTTTTGGCCTAAGAGTCTTTTTATTATTTTTTTTCTTCATAAAA
TCCTTAATTGTTTCATTAACTATCCATGGATTTTTAAGTACGAATCCCTGTTTTTTCAGATGAAATAGAAACTTT
TGAAAGGCTAATACCAGGCTTGTTATGTGCCTTGAGATGTACATATCTACTGGACCCTGAGCATCTCTCTCTAGA
TCTTTTAAATTAAGAAATCACATAGTTTGTGTGACACTGATGCACAAATGATATTTGATAATCTAATTTTACTAA
ATAAGTCATGGACAGTTTCTTGTCTGAATAGGCCACAAAAACTTTAATGTGGGAGAGAACATTTTTTAAACACTC
TAGTGGGGTTTCCATGAGCAAAGGGTGCTTGCAGAAAGTCTCTGATAGTAGAGATGAAGGCTAGGCAGACACCTG
CTGTTTCACCCGCTAAGCTGATGGAGTAACCATGGCAACTGCCACCATATTGTTCTCTTTTCTGAGGACAGATGC
TAATCAGTACAGGTGCTTTCAGAAGAGACTAGGGTATCTATATAGCCTGGTTTATGGATAGGAGAGGTGGTCTTG
GAAACTAAGCCTGGGGTAGTATTCAAGATTGCAATACACTGAAAACTAATTATTGTCTTGTTTTTACAATCTATG
TTAGTAAACTACCAATGACATTGTTCAGTTTAAGTTTTGGGTGTAATCTTCAATACTGACCGGAAAACATCCAGG
TTAGTTATGAAAAGGCAATATGACAGAAAGCCACTTTTGTGTGCTGAGAGTACAACCCGAGATCGTGTGTATTCT
AGGCAAGCACTCTACCACCGACCTACATCTCCAGCCCTTCTGCCTGTGGTTCTTTGTCTTGTAAAGCAATGTCTT
GTTGTTTAGCTGATGCTGGCCTTGCACTTGCTATGTAGCCTTCATCTGCTGGCTGCTAGGACTGCAGATTTGTAC
CACCAATACCGGACTGCAAACCCACTAATTTCTAATGATGAAGGCATGTTTGTATAGAGAGCTGGACTCCTTTCT
TCTGTAGTATACAGGGAGGAAAGAGAAAACAACAAAAACAGCAGCAGCAGCAGCAGCAACAACAACAACAACAAA
AACCCCAAGGACAAGGAAAGTGTTAAGTGAAGGAAAGAAGGGAGGCAGAAGAGGTGGCAGGGAAGCAGGGGAAGC
CCACAGAAGTTAAAGCAGGGTTGTCTCAACCCAGAGAGGAAATGACCCTGGTGCCCTCAGCTCTGTGGCTTCCTT
GACTGATGTATACACCACTCTACCACAGTGATGCCAGGAAAAGGGTGACCAATGCATTGACCTGAGGTTCAACTG
CTCCTGGTTGACAGAGGTACGCTTATAAATAAGTAGGTAGGAAAATTTTGAAGCTTACTTTGAGAGATGAGGCAA
GGTTCTGCACCTCAAGCTCCAGGAATGTCTCGACTGCCATTCACTATGTTTCCTGCGTGATATAGTTCTATTATC
ACCAAAGAAGGCGCTGTACTGACATGTAGGCTACCCCCTTTTCTTACTGCAGGGGAGAATAAATGAAAGGAAGAA
TTATTTGCCAAAATGACACATTTTATGAGAGCCAGATCTTCTTTTTGCTATACCAGTATTCTCCTTGCCATAGCC
AACTGTCTTCAATAAACTATCAATAAGGGGATCTTGGAGAGTGACTGACTACAGCTGAAAGATGGGAAGTGGAGT
GCCAGGGTGGATGGGTGGAGAGGCAAAGGGTGAAGGGAGTGATGAGTTTGTTGAGGGGTGAGCTTGCAGGAGTTC
ATCCAAGATGAACCTCCCCTGGCCTCAGGTGTGGCCTAATAGTTCAAACCGTGGATGATCATGAGCCCACTAAGT
GCCCTTTGGACTTTCCATGTCAGCCATACAGGTGACCCAACCTTCAGTGGTGTTGGCTAGCAGCCATGGTGTCGC
CAGCTTTCCATGTGAATATTCACCATCACACAACACTGATGAGGTCCGGGTGACTGTGCTGCGGCAGACAAATGA
CCAAATGACTGAGGTCTGTGCCACGACATTCACAGAGAAGAATACAGTGGGCTTCCTAGATTACCCCTTCTGCAG
TGGTACCTTTAATGAAAGCAGAGTGAACCTCACCATCCAAGGACTGAGAGCTGTTGACACGGGACTGTACCTCTG
CAAGGTGGAACTCATGTACCCACCGCCATACTTTGTGGGCATGGGCAACGGGACGCAGATTTATGTCATTGGTGA
GCAAAGCCATTCCACTAAGAACAAGTCTGTTGCATTATTATTGTCTTTACACCAGAATAGTTTTGTTCCTTGGTT
TGGAGTCCTTCATAGTTAGGTCTGTGATGCATAGCTAGGAATTCCCTAGTAGATAGTAGTCTTGCTTATACTGAG
AAGTTACATAACCATCACTCTGATTGCAATGAAACGCATTTGGGGATGTGTTTTTTATACTGCTTGCTGATAGTC
TAGGACACTTGTTCTTGAAGTTTAGTCTTGTCCCTTTGATGGCACTCTGGGAAAGTCATGTATTAAATAAGTAGC
CAGACTTCCCTATAGTTTACCAATACAAGTTAGGGTTGACTAGCAAAACCTGGAACCTCTAACTTCCTTTTACTA
CCCATGAGGAACTAGGACCCCACAATTGGAAACTCTCTCAGGAGGTTGATGCTTCGTCTTCTGTTGCAGATCCAG
AACCATGCCCGGATTCTGACTTCCTCCTTTGGATCCTTGTCGCAGTTAGCTTGGGGTTGTTTTTTTACAGTTTCC
TGGTCACTGCTGTTTCTTTGAGCAAGATGGTGAGTGTGATGTTGACGTTTCCCCACAGTTAATGGGGATACTTTT
AGTTGTACCCTACTGACCAATTGGTGTTGAGTTGAAGCAATAAACAAGGAGCAGGAAGGATAGGGTAAAGAACAC
GCTAGAACCCCATGCACTTGCCTTAGAGGTTTCGGGATGACTAATACTGTACGTGAGCATGTTTGACAGTGAATG
TTTGTGTGCTTCTGAGCAGGGTTTCAGTTTGAGTAACTGTTTGAACAACATGGAGCAGCTGTTTTGGTTGTCACT
GTCATGGCAATGTCCTTAATCCTAGGACACACAGCAGTCTCTGGGCAACCCTTTCTAGTTAGAACCACCTAGATG
GATTTTTGTCCTTTACCAAGCACCATCTCTTGGTCCCTCTTGTGTCTTCTTTGTTCAAGGCATAGATACTTGTAG
AATACCCATCCCCATTATCTATGTACTTACTTACCTTGCCTTGATGCCCTCCCTAGCTCAGTGAATGAAAGCCCT
TGTTTTTATTTAGTATATTGGCAGTGAAAAGAGAGAAGTGTAGAATCAAGTCTCCAGTCAGGGTTCTGAACAGTC
CTCCACCATGTCTGTGTAAGGCTGGCTTTCCTTATCACAAGCAATATCCTTTGACCCCATCTCTTCTCTTCTTTT
TCTTACTCTTCCTACTCCTCCATTTTCCTCATCTCTCTACCTTTGCCACTTTCTGTATCTCTTTCTGCATCTATA
TTCACACACACACACACACACACACACACACACACACACACACACACACATACACACACACTTTCACAGAATCTT
TGTTCTGTTGCTGTCTTCCTTTTTTATCTACCATTAAGCTACCACCCAGGCTGGCTGACTAGACATCCATTGTTG
AAGTGTTGAGGTGACAGAGACTGGAAACTAACACAGACAAAGAAAAAGCACTAACAAACATAAGAGGAATGGAGA
TGCAGAATTCAGCTTCAAAGCAGGTTATCAGAGCCACCTTCAAATATTTCAGGTGCTTTGATTACCACAGACACG
ATCTAGCCAACTATCTTGGGCATTTTGGTTGTTCTTGGTGAAGTTGTCTCAAAGCCCCTTTCTCCCTTTTGCAGA
ATTATTTATCTAAGGTGAATTTGACTAGCTCAGTTAAGAAAATCTCCCAGAGTTTATAATTCTGTATGTCGTTGT
GGATGTTTTATTTTTGCTGGGGAGGGACCCGATATTTACTGGGTCCTATTATGATTGCAATTGGCTTCTATATTC
TTTGATAATAATTGCCTTTTAAAAAATGTTTTTTGCAGCTAAAGAAAAGAAGTCCTCTTACAACAGGGGTCTATG
TGAAAATGCCCCCAACAGAGCCAGAATGTGAAAAGCAATTTCAGCCTTATTTTATTCCCATCAACTGAAAGGCCG
TTTATGAAGAAGAAGGAGCATACTTCAGTCTCTAAAAGCTGAGGCAATTTCAACTTTCCTTTTCTCTCCAGCTAT
TTTTACCTGTTTGTATATTTTAAGGAGAGTATGCCTCTCTTTAATAGAAAGCTGGATGCAAAATTCCAATTAAGC
ATACTACAATTTAAAGCTAAGGAGCATGAACAGAGAGCTGGGATATTTCTGTTGTGTCAGAACCATTTTACTAAA
AGCATCACTTGGAAGCAGCATAAGGATATAGCATTATGGTGTGGGGTCAAGGGAACATTAGGGAATGGCACAGCC
CAAAGAAAGGAAGGGGGTGAAGGAAGAGATTATATTGTACACATCTTGTATTTACCTGAGAGATGTTTATGACTT
AAATAATTTTTAAATTTTTCATGCTGTTATTTTCTTTAACAATGTATAATTACACGAAGGTTTAAACATTTATTC
ACAGAGCTATGTGACATAGCCAGTGGTTCCAAAGGTTGTAGTGTTCCAAGATGTATTTTTAAGTAATATTGTACA
TGGGTGTTTCATGTGCTGTTGTGTATTTGCTGGTGGTTTGAATATAAACACTATGTATCAGTGTCGTCCCACAGT
GGGTCCTGGGGAGGTTTGGCTGGGGAGCTTAGGACACTAATCCATCAGGTTGGACTCGAGGTCCTGCACCAACTG
GCTTGGAAACTAGATGAGGCTGTCACAGGGCTCAGTTGCATAAACCGATGGTGATGGAGTGTAAACTGGGTCTTT
ACACTCATTTTATTTTTTGTTTCTGCTTTTGTTTTCTTCAATGATTTGCAAGGAAACCAAAAGCTGGCAGTGTTT
GTATGAACCTGACAGAACACTGTCTTCAAGGAAATGCCTCATTCCTGAGACCAGTAGGTTTGTTTTTTTAGGAAG
TTCCAATACTAGGACCCCCTACAAGTACTATGGCTCCTCGAAAACACAAAGTTAATGCCACAGGAAGCAGCAGAT
GGTAGGATGGGATGCACAAGAGTTCCTGAAAACTAACACTGTTAGTGTTTTTTTTTTAACTCAATATTTTCCATG
AAAATGCAACCACATGTATAATATTTTTAATTAAATAAAAGTTTCTTGTGATTGTTTT

In the methods of the present invention, the site-directed nuclease is capable of cleaving a target nucleotide sequence at the 3′-end of intron 1 of a CTLA4 gene.

An illustrative intron 1 of human CTLA4 may comprise or consist of nucleotides 282-2815 of SEQ ID No: 1. The intron 1 of CTLA4 may comprise or consist of a sequence which is at least 70%, at least 80%, at least 90%, at least 95%, at least 99% or 100% identical to nucleotides 282-2815 of SEQ ID No: 1. An illustrative sequence of intron 1 of human CTLA4 is shown as SEQ ID No: 2. The intron 1 of CTLA4 may comprise or consist of a sequence which is at least 70%, at least 80%, at least 90%, at least 95%, at least 99% or 100% identical to SEQ ID No: 2.

An illustrative intron 1 of mouse CTLA4 may comprise or consist of nucleotides 256-3398 of SEQ ID No: 6. The intron 1 of CTLA4 may comprise or consist of a sequence which is at least 70%, at least 80%, at least 90%, at least 95%, at least 99% or 100% identical to nucleotides 256-3398 of SEQ ID No: 6. An illustrative sequence of intron 1 of mouse CTLA4 is shown as SEQ ID No: 7. The intron 1 of CTLA4 may comprise or consist of a sequence which is at least 70%, at least 80%, at least 90%, at least 95%, at least 99% or 100% identical to SEQ ID No: 7.

Suitably, the 3′-end of intron 1 may refer to the nucleotides located in the downstream half of the intron.

The 3′-end of intron 1 may comprise nucleotides 1268-2534 of SEQ ID No: 2. The 3′-end of intron 1 may comprise a sequence which is at least 70%, at least 80%, at least 90%, at least 95%, at least 99% or 100% identical to nucleotides 1268-2534 of SEQ ID No: 2. The 3′-end of intron 1 may consist of nucleotides 1268-2534 of SEQ ID No: 2. The 3′-end of intron 1 may consist of a sequence which is at least 70%, at least 80%, at least 90%, at least 95%, at least 99% or 100% identical to nucleotides 1268-2534 of SEQ ID No: 2.

For example, the 3′-end of intron 1 may comprise nucleotides 1550-2400, 1900-2330 or 2097-2116 of SEQ ID No: 2. The 3′-end of intron 1 may comprise a sequence which is at least 70%, at least 80%, at least 90%, at least 95%, at least 99% or 100% identical to nucleotides 1550-2400, 1900-2330 or 2097-2116 of SEQ ID No: 2. The 3′-end of intron 1 may consist of nucleotides 1550-2400, 1900-2330 or 2097-2116 of SEQ ID No: 2. The 3′-end of intron 1 may consist of a sequence which is at least 70%, at least 80%, at least 90%, at least 95%, at least 99% or 100% identical to nucleotides 1550-2400, 1900-2330 or 2097-2116 of SEQ ID No: 2.

The 3′-end of intron 1 may comprise nucleotides 1573-3143 of SEQ ID No: 7. The 3′-end of intron 1 may comprise a sequence which is at least 70%, at least 80%, at least 90%, at least 95%, at least 99% or 100% identical to nucleotides 1573-3143 of SEQ ID No: 7. The 3′-end of intron 1 may consist of nucleotides 1573-3143 of SEQ ID No: 7. The 3′-end of intron 1 may consist of a sequence which is at least 70%, at least 80%, at least 90%, at least 95%, at least 99% or 100% identical to nucleotides 1573-3143 of SEQ ID No: 7.

Intron 1 of human CTLA4 (SEQ ID No: 2) with 3′-end highlighted in underline:
GTGAGTGAGACTTTTGGAGCATGAAGATGGAGGAGGTGTTTCTCCTACCTGGGTTTCATTTGTTTCAGCAGTCAA
AGGCAGTGATTTATAGCAAAGCCAGAAGTTAAAGGTAAAACTCCAATCTGGCTTGGCTGGCTCTGTATTCCAGGG
CCAGCAGGGAGCAGTTGGGCGGCAGCAAATAAGGCAAAGAGATAGCTCAGAACAGAGCGCCAGGTATTTAGTAGG
GGCTTCATGAATGCATGTGAGTTGGTTTAGTAGAGAGACACAGGCAATTTCAGACCCTTCTATGAGACTGGAAGT
GATTTAAGAGGGAAAGGATAGCCATAGTCCTGAATACATTTGAGCTGGGTTTCAGGATGAGCTCACAAGTTCCTT
TAAAAAAAATTGACTTAAGCAAATCCTGGGAAGAGTTTTTTTGCTATACAATTCAAGGTTTTAAGGTCCTCGGAT
TCATATACTTTATAAATGAATTAGCCAGCTTGTTTAAAATGTAGGGAAATTGTGGGAAGAATGCCTTCTTTACTT
AATTCAAGGTTTTAAGGTTCTCTTAATCAATTCTACTAGCTAATTAGCCAATTATTTAAAAATAAAAGTTTGAAA
TTGCCAAAAAAAAAAGACAAGGAAAAGGAAAGAAAGAAAGCCACCAGTCTGTTTGGCATACAATACTTAATTGTT
GCCTGACCTACGTGTGGGTTTCAGATGCAGATCCTCAGTTTTCAGCTCTTCAGAGACTGACACCAGGTTTGTTAC
ACGGCTTAAAATGATGAGTATATCCATTGAATCTCAACCTTATCTCTCTCTAGACCTTCTTGGTTAAGAAACCAT
GTAGTTTGTATGAAGTAGGTACTCAAAAGATATTTGATGATTTAATTTTTACTGGAGAAGAAATATTCATATATG
TTTTCTTATTTTTACATGTTTTAAATATGTAAAGATTAAATAAACACTCTTAGAAGTATTTAAATTTCCTAAAGT
AAATTTATCTCAACCAGTAACAGGACCCTCCCAATACTGGAAAGTTGAGTGTGACCGCATTTAGTGGTGATGAGT
GTGAGCTTGCTTGGGGAGAGGGCAGGACATTTAGGATTTCTTAAGCTTAGAGTCAATACAATAAAGATTATTGAG
TGCTCACTTGGGTGGGCTATAATCACTGCTCACAGGAGTTCATGAACCACAAGTAAAAGAGTGAGGAGATATGAT
TAGCTCACAAATAACTTTAATACAGAGCAGAAAGTAATGAACTACTGCAATGGAGTTATCACAGTGCTAAGGATG
CTCAGAGGGCATCTCTGATAGGCAGAGGTGAGGGTTAGGGAAGGAAGCTGTAGTCTAGCTAGCTAGAGCTGCTGG
AATAGACATGACAATGGCTGCTGCCAAACTGTTTTCTCTTCTGAGGACAGATGTCCCGTGCAAGTGGCTTGGTGG
AAGGGACTAGTGTCTCTAATATAGGGTGATTTATAAGCAGGAAAGTGTGTCCTAGAAATTCAGACCAGAGTGATA
GATTGGAATTGGATCATGGGGGACTCATTGAATGTTATTTATTGTATTTGTTTTTGCGATCAGTGTTAGTAAAGT
GTCAAAGGGATTGAGCAGATGAGTGACATCATGCAACACAAGTTTTGAGTTTCACTTGTCAGACTGACTGGAGAG
GGGCCTGGTTAGTTACAGGAAGGTAATTTGGCATGCAGCCACTATTTTTGAGTTGATGCAAGCCTCTCTGTATGG
AGAGCTGGTCTCCTTTATCCTGTGGGAAAAGAGAACAAAGGAGCATGGGAGTGTTCAAGGGAAGGAGAAATAAAG
GGCAGAGAGGCAGCGGTGGTGTCAGGGGAAGCCCACAGGAGTTAACAGCAGGGTTGCCTCAACCTAGAGAGGAAG
CGACCTGGTGCCCTCGGCTCTGTGGCTTCCTTCATCTAACAACATCTTCCACTCTACAACAATGCCAGGGAAGGC
GGAGGCTGGTACAGTGCATCAAGACACAGCTACTCCTGGGTGACAGAGGTTCAGGGCCAGCTCACTAAGTAGGCA
GAAGTTTTTGACATATACTTTGAGAGATAAAGCAAGATTCTGTACCTCAACCTTCAGAATTTCCCCTACCACTCA
TTATAGTTCCGGAGCTATATAGCTCCTATCATTCTATCATAACCTTAGAATACCAGAGAACATATCATCTCATCT
AATTATCTCTTACTATATGTGAAAAAAATGAAGGACATGGGGGAAGTGTGACTTGCCCCAAATCACATATTTCAT
GGTAGAGCCAGGTCTTCTGTTTGTCATATCAGTGTTCTTCCTGCCACAACCATCTTGAAGAATCTATTTCTCAGT
AAGAAAATATCTTTATGGAGAGTAGCTGGAAAACAGTTGAGAGATGGAGGGGAGGCTGGGGGTGTGGAGAGGGGA
AGGGGTAAGTGATAGATTCGTTGAAGGGGGGAGAAAAGGCCGTGGGGATGAAGCTAGAAGGCAGAAGGGCTTGCC
TGGGCTTGGCCATGAAGGAGCATGAGTTCACTGAGTTCCCTTTGGCTTTTCCATGCTAG
Intron 1 of mouse CTLA4 (SEQ ID No: 7) with 3′-end highlighted in underline:
GTGAGTGGAGACTTCTGGAACATGGAGGTGGAGGAGGTGTTTTTCCTACATGGGTTTCGTTTTTTTTTTTTCTTT
CAGAAGCTGAGGACAGTGATTTATAGAAATGCCAGAAGTTAAAGCCAAACCCTCTACTTTTTGGTTTAACAGCTT
CCTACAGACAAGTTCCAAGAAGAATATAGGAGGTCTAGCAGTAGCGAAGGAGCTGAGACTAGCCACAGCAGCTTG
CTGGGATCTCAGCTGGGACTTAAGGAACCATGAGAGTTCCTTAAGTCCCATATGGGGGAGAACAAGTTCTTTGTC
ACTGCTAGAAAATCCTGTAAAGTTAAGAGATTTAAGGGGGGCATTTGAAATCGTGAGTACATTTGATCTTGATAT
CACAGTAACATTAAGGTTTTTCTCCTTACCCCCAATCCCACCCCCAAAACTTACCTAAATTCAAATAAATAAGAA
GTTTTCCTCATTCACTTTAAGATTTTGAGACACTCTTAGTTCTTTAAATTATCACACCTAAGGCTTAGGGGACAT
AGCAGAAGAGGGGGCAGAAAGACTATGAGAGCCAAAGGTACATAGAGTTTACTGAGAGATTGTGTCTCTTAGGAT
GTCAAATGGTACACCCATAAAGTCTCACCAACACGACTGCCTAAACTTGAGCTGAATAAGGACATGAATAACAAT
AGACATACCCAAGTGGATGGGGAAAGACCAAGATGCTTCAGCACTATAGCAATAACTACAGGAAGCCAAGGAATA
TGGAGAGTAGGAAATTGCCAAGGGAAAAGCACACCAGCTGACTATCCAATAACATATGGTCAGCCTTCAAAACAA
AAAGCAAATAACATATAGAATGGGCAGGTTTCATTAAGAATGACTATAACTATAGTCTGTGCATATCACAACAAA
TAATGAAAAGGGGGGCCATGAATTTGAAAGAAAAGCAAGGAGGGGTACATGAGAAGGTTTGGAGGAGAGAAAGGA
GAAATAATGTAATTATATTAATCTCAAAGAATAGAAGAAGTAGTAAGTAAAGGGAAAATAAATTAGTTAATTATT
TTAAAGATGGAATAGTTTTAAGTCGAAGGAAGAAAGAAAGGCAAAGTGATAGAAAGACAAGAAAAGGGGAGGAAG
GAAGAGAGAAGAGAGGAAGGAAGAAAGGAAGGAAGGAAGGAAGGGAGGAAGGGAGGGAAAAAGAAAGAAGAGAGA
AGGGAGGAGGACAGGAAGGAGGGAGGGAGGGAGGGAGGAAGGGAGAAAGGGAGAAAGGAAGAAAACAATTATCAG
CTTTTTGGCCTAAGAGTCTTTTTATTATTTTTTTTCTTCATAAAATCCTTAATTGTTTCATTAACTATCCATGGA
TTTTTAAGTACGAATCCCTGTTTTTTCAGATGAAATAGAAACTTTTGAAAGGCTAATACCAGGCTTGTTATGTGC
CTTGAGATGTACATATCTACTGGACCCTGAGCATCTCTCTCTAGATCTTTTAAATTAAGAAATCACATAGTTTGT
GTGACACTGATGCACAAATGATATTTGATAATCTAATTTTACTAAATAAGTCATGGACAGTTTCTTGTCTGAATA
GGCCACAAAAACTTTAATGTGGGAGAGAACATTTTTTAAACACTCTAGTGGGGTTTCCATGAGCAAAGGGTGCTT
GCAGAAAGTCTCTGATAGTAGAGATGAAGGCTAGGCAGACACCTGCTGTTTCACCCGCTAAGCTGATGGAGTAAC
CATGGCAACTGCCACCATATTGTTCTCTTTTCTGAGGACAGATGCTAATCAGTACAGGTGCTTTCAGAAGAGACT
AGGGTATCTATATAGCCTGGTTTATGGATAGGAGAGGTGGTCTTGGAAACTAAGCCTGGGGTAGTATTCAAGATT
GCAATACACTGAAAACTAATTATTGTCTTGTTTTTACAATCTATGTTAGTAAACTACCAATGACATTGTTCAGTT
TAAGTTTTGGGTGTAATCTTCAATACTGACCGGAAAACATCCAGGTTAGTTATGAAAAGGCAATATGACAGAAAG
CCACTTTTGTGTGCTGAGAGTACAACCCGAGATCGTGTGTATTCTAGGCAAGCACTCTACCACCGACCTACATCT
CCAGCCCTTCTGCCTGTGGTTCTTTGTCTTGTAAAGCAATGTCTTGTTGTTTAGCTGATGCTGGCCTTGCACTTG
CTATGTAGCCTTCATCTGCTGGCTGCTAGGACTGCAGATTTGTACCACCAATACCGGACTGCAAACCCACTAATT
TCTAATGATGAAGGCATGTTTGTATAGAGAGCTGGACTCCTTTCTTCTGTAGTATACAGGGAGGAAAGAGAAAAC
AACAAAAACAGCAGCAGCAGCAGCAGCAACAACAACAACAACAAAAACCCCAAGGACAAGGAAAGTGTTAAGTGA
AGGAAAGAAGGGAGGCAGAAGAGGTGGCAGGGAAGCAGGGGAAGCCCACAGAAGTTAAAGCAGGGTTGTCTCAAC
CCAGAGAGGAAATGACCCTGGTGCCCTCAGCTCTGTGGCTTCCTTGACTGATGTATACACCACTCTACCACAGTG
ATGCCAGGAAAAGGGTGACCAATGCATTGACCTGAGGTTCAACTGCTCCTGGTTGACAGAGGTACGCTTATAAAT
AAGTAGGTAGGAAAATTTTGAAGCTTACTTTGAGAGATGAGGCAAGGTTCTGCACCTCAAGCTCCAGGAATGTCT
CGACTGCCATTCACTATGTTTCCTGCGTGATATAGTTCTATTATCACCAAAGAAGGCGCTGTACTGACATGTAGG
CTACCCCCTTTTCTTACTGCAGGGGAGAATAAATGAAAGGAAGAATTATTTGCCAAAATGACACATTTTATGAGA
GCCAGATCTTCTTTTTGCTATACCAGTATTCTCCTTGCCATAGCCAACTGTCTTCAATAAACTATCAATAAGGGG
ATCTTGGAGAGTGACTGACTACAGCTGAAAGATGGGAAGTGGAGTGCCAGGGTGGATGGGTGGAGAGGCAAAGGG
TGAAGGGAGTGATGAGTTTGTTGAGGGGTGAGCTTGCAGGAGTTCATCCAAGATGAACCTCCCCTGGCCTCAGGT
GTGGCCTAATAGTTCAAACCGTGGATGATCATGAGCCCACTAAGTGCCCTTTGGACTTTCCATGTCAG

A target nucleotide sequence refers to a sequence that is recognised (i.e. bound) by a site-directed nuclease. The site-directed nuclease may act on (i.e. cleave) a site between two nucleotides within the target sequence. For example, the site-directed nuclease may cause a double strand break within the target sequence. Thus, the target sequence directs the position of the cleavage.

For example, the target nucleotide sequence may comprise or consist of around 5-50, 10-40, or 20-30 nucleotides within the sequence corresponding to the 3′-end of intron 1 of CTLA4.

Preferably, the target sequence is unique compared to the rest of the genome. The target sequence may be located immediately adjacent to a Protospacer Adjacent Motif (PAM), i.e. the target sequence may be located at the 5′-end of the PAM.

For example, the target sequence may comprise the sequence AGCTCCGGAACTATAATGAG (SEQ ID No: 12), immediately upstream of the PAM sequence TGG. This sequence is complementary to the sequence CTCATTATAGTTCCGGAGCT (SEQ ID No: 13), which corresponds to nucleotides 2097-2116 of SEQ ID No: 2.

The target sequence may comprise the sequence GAGAAATAGATTCTTCAAGA (SEQ ID No: 15), immediately upstream of the PAM sequence TGG. This sequence is complementary to the sequence TCTTGAAGAATCTATTTCTC (SEQ ID No: 16), which corresponds to nucleotides 2303-2322 of SEQ ID No: 2.

The target sequence may comprise the sequence GATATGACAAACAGAAGACC (SEQ ID No: 18), immediately upstream of the PAM sequence TGG. This sequence is complementary to the sequence GGTCTTCTGTTTGTCATATC (SEQ ID No: 19), which corresponds to nucleotides 2261-2280 of SEQ ID No: 2.

### Nucleic Acid Construct

In order to utilise HDR for gene insertion, a repair template must be delivered into the cell. The repair template should contain the desired gene insertion as well as additional homologous sequences immediately upstream and downstream of the target (5′ and 3′ homology arms).

Thus, the present invention provides a nucleic acid construct, which comprises a nucleic acid sequence comprising replacement CTLA4 sequences flanked by 5′ and 3′ homology arms.

The nucleic acid construct of the invention may comprise a nucleotide sequence comprising one or more of exon 2, exon 3 and/or exon 4 of the CTLA4 gene, or a sequence with at least 70% identity to exon 2, exon 3 and/or exon 4 of the CTLA4 gene.

In one embodiment, the nucleic acid construct comprises a nucleotide sequence comprising exons 2, 3 and 4.

In one embodiment, the nucleic acid construct comprises a nucleotide sequence comprising exons 2 and 4.

In one embodiment, the nucleic acid construct comprises a nucleotide sequence comprising exon 4.

In one embodiment, the nucleic acid construct according to the invention comprises one or more of SEQ ID Nos: 3, 4 and/or 5. In one embodiment, the nucleic acid construct according to the invention comprises a nucleotide sequence comprising one or more sequences which are at least 70%, at least 80%, at least 90%, at least 95%, at least 99% or 100% identical to SEQ ID Nos: 3, 4 and/or 5.

In one embodiment, the nucleic acid construct according to the invention comprises one or more of SEQ ID Nos: 8, 9 and/or 10. In one embodiment, the nucleic acid construct according to the invention comprises a nucleotide sequence comprising one or more sequences which are at least 70%, at least 80%, at least 90%, at least 95%, at least 99% or 100% identical to SEQ ID Nos: 8, 9 and/or 10.

It will be apparent to the skilled person that the exons should be ordered such that they will encode a functional CTLA4 protein. For example, the exons may be ordered exon 2-exon 3-exon 4. As described above, one or more exons may be omitted.

The nucleic acid construct may further comprise intronic sequences.

Exon 2 of human CTLA4 (SEQ ID No: 3)-corresponds to nucleotides
2816-3163 of SEQ ID No: 1
CAATGCACGTGGCCCAGCCTGCTGTGGTACTGGCCAGCAGCCGAGGCATCGCCAGCTTTGTGTGTGAGTATGCAT
CTCCAGGCAAAGCCACTGAGGTCCGGGTGACAGTGCTTCGGCAGGCTGACAGCCAGGTGACTGAAGTCTGTGCGG
CAACCTACATGATGGGGAATGAGTTGACCTTCCTAGATGATTCCATCTGCACGGGCACCTCCAGTGGAAATCAAG
TGAACCTCACTATCCAAGGACTGAGGGCCATGGACACGGGACTCTACATCTGCAAGGTGGAGCTCATGTACCCAC
CGCCATACTACCTGGGCATAGGCAACGGAACCCAGATTTATGTAATTG
Exon 3 of human CTLA4 (SEQ ID No: 4)-corresponds to nucleotides
3608-3717 of SEQ ID No: 1
ATCCAGAACCGTGCCCAGATTCTGACTTCCTCCTCTGGATCCTTGCAGCAGTTAGTTCGGGGTTGTTTTTTTATA
GCTTTCTCCTCACAGCTGTTTCTTTGAGCAAAATG
Exon 4 of human CTLA4 (SEQ ID No: 5)-corresponds to nucleotides
4938-6195 of SEQ ID No: 1
CTAAAGAAAAGAAGCCCTCTTACAACAGGGGTCTATGTGAAAATGCCCCCAACAGAGCCAGAATGTGAAAAGCAA
TTTCAGCCTTATTTTATTCCCATCAATTGAGAAACCATTATGAAGAAGAGAGTCCATATTTCAATTTCCAAGAGC
TGAGGCAATTCTAACTTTTTTGCTATCCAGCTATTTTTATTTGTTTGTGCATTTGGGGGGAATTCATCTCTCTTT
AATATAAAGTTGGATGCGGAACCCAAATTACGTGTACTACAATTTAAAGCAAAGGAGTAGAAAGACAGAGCTGGG
ATGTTTCTGTCACATCAGCTCCACTTTCAGTGAAAGCATCACTTGGGATTAATATGGGGATGCAGCATTATGATG
TGGGTCAAGGAATTAAGTTAGGGAATGGCACAGCCCAAAGAAGGAAAAGGCAGGGAGCGAGGGAGAAGACTATAT
TGTACACACCTTATATTTACGTATGAGACGTTTATAGCCGAAATGATCTTTTCAAGTTAAATTTTATGCCTTTTA
TTTCTTAAACAAATGTATGATTACATCAAGGCTTCAAAAATACTCACATGGCTATGTTTTAGCCAGTGATGCTAA
AGGTTGTATTGCATATATACATATATATATATATATATATATATATATATATATATATATATATATATATATATA
TTTTAATTTGATAGTATTGTGCATAGAGCCACGTATGTTTTTGTGTATTTGTTAATGGTTTGAATATAAACACTA
TATGGCAGTGTCTTTCCACCTTGGGTCCCAGGGAAGTTTTGTGGAGGAGCTCAGGACACTAATACACCAGGTAGA
ACACAAGGTCATTTGCTAACTAGCTTGGAAACTGGATGAGGTCATAGCAGTGCTTGATTGCGTGGAATTGTGCTG
AGTTGGTGTTGACATGTGCTTTGGGGCTTTTACACCAGTTCCTTTCAATGGTTTGCAAGGAAGCCACAGCTGGTG
GTATCTGAGTTGACTTGACAGAACACTGTCTTGAAGACAATGGCTTACTCCAGGAGACCCACAGGTATGACCTTC
TAGGAAGCTCCAGTTCGATGGGCCCAATTCTTACAAACATGTGGTTAATGCCATGGACAGAAGAAGGCAGCAGGT
GGCAGAATGGGGTGCATGAAGGTTTCTGAAAATTAACACTGCTTGTGTTTTTAACTCAATATTTTCCATGAAAAT
GCAACAACATGTATAATATTTTTAATTAAATAAAAATCTGTGGTGGTCGTTTTCCGGA
Exon 2 of mouse CTLA4 (SEQ ID No: 8)-corresponds to nucleotides
3399-3746 of SEQ ID No: 6
CCATACAGGTGACCCAACCTTCAGTGGTGTTGGCTAGCAGCCATGGTGTCGCCAGCTTTCCATGTGAATATTCAC
CATCACACAACACTGATGAGGTCCGGGTGACTGTGCTGCGGCAGACAAATGACCAAATGACTGAGGTCTGTGCCA
CGACATTCACAGAGAAGAATACAGTGGGCTTCCTAGATTACCCCTTCTGCAGTGGTACCTTTAATGAAAGCAGAG
TGAACCTCACCATCCAAGGACTGAGAGCTGTTGACACGGGACTGTACCTCTGCAAGGTGGAACTCATGTACCCAC
CGCCATACTTTGTGGGCATGGGCAACGGGACGCAGATTTATGTCATTG
Exon 3 of mouse CTLA4 (SEQ ID No: 9)-corresponds to nucleotides
4195-4304 of SEQ ID No: 6
ATCCAGAACCATGCCCGGATTCTGACTTCCTCCTTTGGATCCTTGTCGCAGTTAGCTTGGGGTTGTTTTTTTACA
GTTTCCTGGTCACTGCTGTTTCTTTGAGCAAGATG
Exon 4 of mouse CTLA4 (SEQ ID No: 10)-corresponds to nucleotides
5589-6808 of SEQ ID No: 6
CTAAAGAAAAGAAGTCCTCTTACAACAGGGGTCTATGTGAAAATGCCCCCAACAGAGCCAGAATGTGAAAAGCAA
TTTCAGCCTTATTTTATTCCCATCAACTGAAAGGCCGTTTATGAAGAAGAAGGAGCATACTTCAGTCTCTAAAAG
CTGAGGCAATTTCAACTTTCCTTTTCTCTCCAGCTATTTTTACCTGTTTGTATATTTTAAGGAGAGTATGCCTCT
CTTTAATAGAAAGCTGGATGCAAAATTCCAATTAAGCATACTACAATTTAAAGCTAAGGAGCATGAACAGAGAGC
TGGGATATTTCTGTTGTGTCAGAACCATTTTACTAAAAGCATCACTTGGAAGCAGCATAAGGATATAGCATTATG
GTGTGGGGTCAAGGGAACATTAGGGAATGGCACAGCCCAAAGAAAGGAAGGGGGTGAAGGAAGAGATTATATTGT
ACACATCTTGTATTTACCTGAGAGATGTTTATGACTTAAATAATTTTTAAATTTTTCATGCTGTTATTTTCTTTA
ACAATGTATAATTACACGAAGGTTTAAACATTTATTCACAGAGCTATGTGACATAGCCAGTGGTTCCAAAGGTTG
TAGTGTTCCAAGATGTATTTTTAAGTAATATTGTACATGGGTGTTTCATGTGCTGTTGTGTATTTGCTGGTGGTT
TGAATATAAACACTATGTATCAGTGTCGTCCCACAGTGGGTCCTGGGGAGGTTTGGCTGGGGAGCTTAGGACACT
AATCCATCAGGTTGGACTCGAGGTCCTGCACCAACTGGCTTGGAAACTAGATGAGGCTGTCACAGGGCTCAGTTG
CATAAACCGATGGTGATGGAGTGTAAACTGGGTCTTTACACTCATTTTATTTTTTGTTTCTGCTTTTGTTTTCTT
CAATGATTTGCAAGGAAACCAAAAGCTGGCAGTGTTTGTATGAACCTGACAGAACACTGTCTTCAAGGAAATGCC
TCATTCCTGAGACCAGTAGGTTTGTTTTTTTAGGAAGTTCCAATACTAGGACCCCCTACAAGTACTATGGCTCCT
CGAAAACACAAAGTTAATGCCACAGGAAGCAGCAGATGGTAGGATGGGATGCACAAGAGTTCCTGAAAACTAACA
CTGTTAGTGTTTTTTTTTTAACTCAATATTTTCCATGAAAATGCAACCACATGTATAATATTTTTAATTAAATAA
AAGTTTCTTGTGATTGTTTT
CTLA4 nucleic acid construct
(SEQ ID No: 31)
CAATGCACGTGGCCCAGCCTGCTGTGGTACTGGCCAGCAGCCGAGGCATCGCCAGCTTTGTGTGTGAGTATGCAT
CTCCAGGCAAAGCCACTGAGGTCCGGGTGACAGTGCTTCGGCAGGCTGACAGCCAGGTGACTGAAGTCTGTGCGG
CAACCTACATGATGGGGAATGAGTTGACCTTCCTAGATGATTCCATCTGCACGGGCACCTCCAGTGGAAATCAAG
TGAACCTCACTATCCAAGGACTGAGGGCCATGGACACGGGACTCTACATCTGCAAGGTGGAGCTCATGTACCCAC
CGCCATACTACCTGGGCATAGGCAACGGAACCCAGATTTATGTAATTGATCCAGAACCGTGCCCAGATTCTGACT
TCCTCCTCTGGATCCTTGCAGCAGTTAGTTCGGGGTTGTTTTTTTATAGCTTTCTCCTCACAGCTGTTTCTTTGA
GCAAAATGCTAAAGAAAAGAAGCCCTCTTACAACAGGGGTCTATGTGAAAATGCCCCCAACAGAGCCAGAATGTG
AAAAGCAATTTCAGCCTTATTTTATTCCCATCAAT

In one embodiment, the nucleic acid construct comprises a nucleotide sequence comprising SEQ ID No: 31 or a variant which is at least 70%, at least 80%, at least 90%, at least 95%, at least 99% identical to SEQ ID No: 31. The variant should enable the production of a functional CTLA4 protein.

The nucleic acid sequence comprising CTLA4 exons may be flanked by one or more homology arms, as described below.

Suitably, further intervening sequences may be present between the nucleic acid sequence comprising CTLA4 exons and the homology arms (for example, splice acceptor sequence or self-cleaving peptide).

The terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Furthermore, the nucleotide sequences comprising exon 2, exon 3 or exon 4 may comprise mutations intended to modulate (e.g. enhance or inhibit) the interaction between CTLA4 and CD80 and/or CD86.

Accordingly, the nucleic acid construct of the present invention may comprise a nucleotide sequence comprising one or more sequences which are at least 70%, 75%, 80%, 85%, 90%, 95% or 99% identical to exon 2, exon 3 and/or exon 4 of a CTLA4 gene (e.g. SEQ ID Nos: 3, 4, 5, 7, 8 and/or 9).

The percentage identity between two nucleotide sequences may be readily determined by programs such as EMBOSS Needle, which is available at https://www.ebi.ac.uk/Tools/psa/. Suitably, the percentage identity is determined across the entirety of the reference and/or the query sequence.

The nucleic acid construct of the invention comprises 5′ and 3′ homology arms, wherein each of the 5′ and 3′ homology arms is essentially complementary to a sequence flanking a target nucleotide sequence at the 3′-end of Intron 1 of CTLA4.

The length of each homology arm is dependent on the size of the change being introduced, with larger insertions requiring longer homology arms. The overall length could be limited by parameters such as plasmid size or viral packaging limits.

For example, the homology arm may include at least 20, 50, 100, 250, 500, 750, or 1000 nucleotides. The homology arm may be 20-1000, 50-750, 100-500, preferably 300-500 nucleotides in length.

The 5′ and 3′ homology arms may be different lengths (asymmetrical).

In one aspect, one homology arm comprises or consists of SEQ ID Nos: 22. In one aspect, the homology arm comprises or consists of a sequence which is at least 70%, at least 80%, at least 90%, at least 95%, at least 99% or 100% identical to SEQ ID No: 22.

In one aspect, one homology arm comprises or consists of SEQ ID Nos: 23. In one aspect, the homology arm comprises or consists of a sequence which is at least 70%, at least 80%, at least 90%, at least 95%, at least 99% or 100% identical to SEQ ID No: 23.

In one aspect, the homology arms comprise or consist of the sequences of SEQ ID Nos: 22 and 23. In one aspect, the homology arms comprise sequences which are at least 70%, at least 80%, at least 90%, at least 95%, at least 99% or 100% identical to SEQ ID Nos: 22 and 23.

In one aspect, the homology arms have the sequences of SEQ ID Nos: 22 and 23. In one aspect, the homology arms comprise sequences which are at least 70%, at least 80%, at least 90%, at least 95%, at least 99% or 100% identical to SEQ ID Nos: 22 and 23.

Sequence of 5′ homology arm
(SEQ ID No: 22)
ATTTTTGAGTTGATGCAAGCCTCTCTGTATGGAGAGCTGGTCTCCTTTATCCTGTGGGAAAAGAGAACAAAGGAG
CATGGGAGTGTTCAAGGGAAGGAGAAATAAAGGGCAGAGAGGCAGCGGTGGTGTCAGGGGAAGCCCACAGGAGTT
AACAGCAGGGTTGCCTCAACCTAGAGAGGAAGCGACCTGGTGCCCTCGGCTCTGTGGCTTCCTTCATCTAACAAC
ATCTTCCACTCTACAACAATGCCAGGGAAGGCGGAGGCTGGTACAGTGCATCAAGACACAGCTACTCCTGGGTGA
CAGAGGTTCAGGGCCAGCTCACTAAGTAGGCAGAAGTTTTTGACATATACTTTGAGAGATAAAGCAAGATTCTGT
ACCTCAACCTTCAGAATTTCCCCT
Sequence of 3′ homology arm
(SEQ ID No: 23)
ATTATAGTTCCGGAGCTATATAGCTCCTATCATTCTATCATAACCTTAGAATACCAGAGAACATATCATCTCATC
TAATTATCTCTTACTATATGTGAAAAAAATGAAGGACATGGGGGAAGTGTGACTTGCCCCAAATCACATATTTCA
TGGTAGAGCCAGGTCTTCTGTTTGTCATATCAGTGTTCTTCCTGCCACAACCATCTTGAAGAATCTATTTCTCAG
TAAGAAAATATCTTTATGGAGAGTAGCTGGAAAACAGTTGAGAGATGGAGGGGAGGCTGGGGGTGTGGAGAGGGG
AAGGGGTAAGTGATAGATTCGTTGAAGGGGGGAGAAAAGGCCGTGGGGATGAAGCTAGAAGGCAGAAGGGCTTGC
CTGGGCTTGGCCATGAAGGAGCATGAGTTCACTGAGTTC

The term “essentially complementary” refers to a sequence that has a degree of identity to a sequence flanking the target sequence that is sufficient to mediate homology directed repair.

For example, each 5′ and 3′ homology arm may be at least 80%, 85%, 90%, 95%, 99% or 100% identical to a sequence flanking the target nucleotide sequence.

In some embodiments, each 5′ and 3′ homology arm may comprise no more than 10, 9, 8, 7, 6, 5, 4, 3, 2, or 1 nucleotide change(s) compared to a sequence flanking the target nucleotide sequence.

The nucleic acid construct of the invention may comprise a splice acceptor (SA) sequence.

The splice acceptor sequence may be located at the 5′-end of the first exon included in the repair template, such that it facilitates joining of the replacement CTLA4 exon sequences with endogenous exon 1. For example, the splice acceptor sequence may be located at the 5′-end of the nucleotide sequence comprising exon 2 or exon 4.

In one aspect, the splice acceptor comprises the sequence ACTGACCTCTTCTCTTCCTCCCACAG (SEQ ID No: 24).

The nucleic acid construct may further comprise a reporter gene sequence. The reporter gene may be used as an indicator of whether the inserted CTLA4 sequences are expressed by the cell. Expression of the reporter gene may confer characteristics to the cell that are easily identified and/or measured. Suitable reporter genes are known to those skilled in the art.

In one embodiment, the reporter gene sequence encodes a green fluorescent protein (GFP).

The reporter gene sequence may be separated from the CTLA4 sequences by a sequence encoding a cleavage site.

The cleavage site may encode a self-cleaving peptide. A ‘self-cleaving peptide’ refers to a peptide which functions such that when the polypeptide comprising the first and second polypeptides (e.g. CTLA4 and GFP) and the self-cleaving peptide is produced, it is immediately “cleaved” or separated into distinct and discrete first and second polypeptides without the need for any external cleavage activity.

In one embodiment, the sequence is a P2A sequence. For example, GGAAGCGGAGCCACCAACTTCTCCCTGCTGAAGCAGGCCGGCGACGTGGAGGAGAAC CCCGGCCCC (SEQ ID No. 25).

The nucleic acid construct of the invention may further comprise regulatory elements. For example, the 3′ untranslated region (3′-UTR) of CTLA4 or a woodchuck hepatitis virus posttranscriptional regulatory element (WPRE), and/or a poly(A) tail. The regulatory elements may improve mRNA stability and protein yield.

In one embodiment, the repair template (nucleic acid construct) has the structure:
[5′ homology arm]-[splice acceptor]-[replacement CTLA4 sequence(s)]-[cleavage site]-[reporter gene]-[regulatory elements]-[3′ homology arm].

In one embodiment, the nucleic acid construct of the invention comprises the sequence SEQ ID No: 20. In one embodiment, the nucleic acid construct of the invention comprises a sequence which is at least 70%, at least 80%, at least 90%, at least 95%, at least 99% or 100% identical to SEQ ID No: 20.

In one embodiment, the nucleic acid construct of the invention comprises the sequence SEQ ID No: 21. In one embodiment, the nucleic acid construct of the invention comprises a sequence which is at least 70%, at least 80%, at least 90%, at least 95%, at least 99% or 100% identical to SEQ ID No: 21.

Sequence of CTLA4-P2A-GFP-WPRE
(SEQ ID No: 20)
GCTTTTAATTAAATTTTTGAGTTGATGCAAGCCTCTCTGTATGGAGAGCTGGTCTCCTTTATCCTGTGGGAAAAG
AGAACAAAGGAGCATGGGAGTGTTCAAGGGAAGGAGAAATAAAGGGCAGAGAGGCAGCGGTGGTGTCAGGGGAAG
CCCACAGGAGTTAACAGCAGGGTTGCCTCAACCTAGAGAGGAAGCGACCTGGTGCCCTCGGCTCTGTGGCTTCCT
TCATCTAACAACATCTTCCACTCTACAACAATGCCAGGGAAGGCGGAGGCTGGTACAGTGCATCAAGACACAGCT
ACTCCTGGGTGACAGAGGTTCAGGGCCAGCTCACTAAGTAGGCAGAAGTTTTTGACATATACTTTGAGAGATAAA
GCAAGATTCTGTACCTCAACCTTCAGAATTTCCCCTACTGACCTCTTCTCTTCCTCCCACAGCAATGCACGTGGC
CCAGCCTGCTGTGGTACTGGCCAGCAGCCGAGGCATCGCCAGCTTTGTGTGTGAGTATGCATCTCCAGGCAAAGC
CACTGAGGTCCGGGTGACAGTGCTTCGGCAGGCTGACAGCCAGGTGACTGAAGTCTGTGCGGCAACCTACATGAT
GGGGAATGAGTTGACCTTCCTAGATGATTCCATCTGCACGGGCACCTCCAGTGGAAATCAAGTGAACCTCACTAT
CCAAGGACTGAGGGCCATGGACACGGGACTCTACATCTGCAAGGTGGAGCTCATGTACCCACCGCCATACTACCT
GGGCATAGGCAACGGAACCCAGATTTATGTAATTGATCCAGAACCGTGCCCAGATTCTGACTTCCTCCTCTGGAT
CCTTGCAGCAGTTAGTTCGGGGTTGTTTTTTTATAGCTTTCTCCTCACAGCTGTTTCTTTGAGCAAAATGCTAAA
GAAAAGAAGCCCTCTTACAACAGGGGTCTATGTGAAAATGCCCCCAACAGAGCCAGAATGTGAAAAGCAATTTCA
GCCTTATTTTATTCCCATCAATGGAAGCGGAGCCACCAACTTCTCCCTGCTGAAGCAGGCCGGCGACGTGGAGGA
GAACCCCGGCCCCATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATGCCACCTACGGCAAGCTGACCCTGAA
GTTCATCTGCACCACCGGCAAGCTGCCCGTGCCCTGGCCCACCCTCGTGACCACCCTGACCTACGGCGTGCAGTG
CTTCAGCCGCTACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTGGAGTACAACTA
CAACAGCCACAACGTCTATATCATGGCCGACAAGCAGAAGAACGGCATCAAGGTGAACTTCAAGATCCGCCACAA
CATCGAGGACGGCAGCGTGCAGCTCGCCGACCACTACCAGCAGAACACCCCCATCGGCGACGGCCCCGTGCTGCT
GCCCGACAACCACTACCTGAGCACCCAGTCCGCCCTGAGCAAAGACCCCAACGAGAAGCGCGATCACATGGTCCT
GCTGGAGTTCGTGACCGCCGCCGGGATCACTCTCGGCATGGACGAGCTGTACAAGTAAAATCAACCTCTGGATTA
CAAAATTTGTGAAAGATTGACTGGTATTCTTAACTATGTTGCTCCTTTTACGCTATGTGGATACGCTGCTTTAAT
GCCTTTGTATCATGCTATTGCTTCCCGTATGGCTTTCATTTTCTCCTCCTTGTATAAATCCTGGTTGCTGTCTCT
TTATGAGGAGTTGTGGCCCGTTGTCAGGCAACGTGGCGTGGTGTGCACTGTGTTTGCTGACGCAACCCCCACTGG
TTGGGGCATTGCCACCACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATTCCGTGGTGTTGTCGGG
GAAATCATCGTCCTTTCCTTGGCTGCTCGCCTGTGTTGCCACCTGGATTCTGCGCGGGACGTCCTTCTGCTACGT
CCCTTCGGCCCTCAATCCAGCGGACCTTCCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGCCTGAAGATACATTGATGAGTTTGGACAA
ACCACAACTAGAATGCAGTGAAAAAAATGCTTTATTTGTGAAATTTGTGATGCTATTGCTTTATTTGTAACCATT
ATAAGCTGCAATAAACAAGTTATTATAGTTCCGGAGCTATATAGCTCCTATCATTCTATCATAACCTTAGAATAC
CAGAGAACATATCATCTCATCTAATTATCTCTTACTATATGTGAAAAAAATGAAGGACATGGGGGAAGTGTGACT
TGCCCCAAATCACATATTTCATGGTAGAGCCAGGTCTTCTGTTTGTCATATCAGTGTTCTTCCTGCCACAACCAT
CTTGAAGAATCTATTTCTCAGTAAGAAAATATCTTTATGGAGAGTAGCTGGAAAACAGTTGAGAGATGGAGGGGA
GGCTGGGGGTGTGGAGAGGGGAAGGGGTAAGTGATAGATTCGTTGAAGGGGGGAGAAAAGGCCGTGGGGATGAAG
CTAGAAGGCAGAAGGGCTTGCCTGGGCTTGGCCATGAAGGAGCATGAGTTCACTGAGTTCCCTAGGCATGTCTCA
TGCTAGCTCTCGAT
Sequence of CTLA4-P2A-GFP-3′UTR
(SEQ ID No: 21)
GTTTAAACCCTGCAGGATTTTTGAGTTGATGCAAGCCTCTCTGTATGGAGAGCTGGTCTCCTTTATCCTGTGGGA
AAAGAGAACAAAGGAGCATGGGAGTGTTCAAGGGAAGGAGAAATAAAGGGCAGAGAGGCAGCGGTGGTGTCAGGG
GAAGCCCACAGGAGTTAACAGCAGGGTTGCCTCAACCTAGAGAGGAAGCGACCTGGTGCCCTCGGCTCTGTGGCT
TCCTTCATCTAACAACATCTTCCACTCTACAACAATGCCAGGGAAGGCGGAGGCTGGTACAGTGCATCAAGACAC
AGCTACTCCTGGGTGACAGAGGTTCAGGGCCAGCTCACTAAGTAGGCAGAAGTTTTTGACATATACTTTGAGAGA
TAAAGCAAGATTCTGTACCTCAACCTTCAGAATTTCCCCTAACTGACCTCTTCTCTTCCTCCCACAGCAATGCAC
GTGGCCCAGCCTGCTGTGGTACTGGCCAGCAGCCGAGGCATCGCCAGCTTTGTGTGTGAGTATGCATCTCCAGGC
AAAGCCACTGAGGTCCGGGTGACAGTGCTTCGGCAGGCTGACAGCCAGGTGACTGAAGTCTGTGCGGCAACCTAC
ATGATGGGGAATGAGTTGACCTTCCTAGATGATTCCATCTGCACGGGCACCTCCAGTGGAAATCAAGTGAACCTC
ACTATCCAAGGACTGAGGGCCATGGACACGGGACTCTACATCTGCAAGGTGGAGCTCATGTACCCACCGCCATAC
TACCTGGGCATAGGCAACGGAACCCAGATTTATGTAATTGATCCAGAACCGTGCCCAGATTCTGACTTCCTCCTC
TGGATCCTTGCAGCAGTTAGTTCGGGGTTGTTTTTTTATAGCTTTCTCCTCACAGCTGTTTCTTTGAGCAAAATG
CTAAAGAAAAGAAGCCCTCTTACAACAGGGGTCTATGTGAAAATGCCCCCAACAGAGCCAGAATGTGAAAAGCAA
TTTCAGCCTTATTTTATTCCCATCAATGGAAGCGGAGCCACCAACTTCTCCCTGCTGAAGCAGGCCGGCGACGTG
GAGGAGAACCCCGGCCCCATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCCACCTACGGCAAGCTGACC
CTGAAGTTCATCTGCACCACCGGCAAGCTGCCCGTGCCCTGGCCCACCCTCGTGACCACCCTGACCTACGGCGTG
CAGTGCTTCAGCCGCTACCCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGTGAAGTTCGAGGGCGACACC
CTGGTGAACCGCATCGAGCTGAAGGGCATCGACTTCAAGGAGGACGGCAACATCCTGGGGCACAAGCTGGAGTAC
AACTACAACAGCCACAACGTCTATATCATGGCCGACAAGCAGAAGAACGGCATCAAGGTGAACTTCAAGATCCGC
CACAACATCGAGGACGGCAGCGTGCAGCTCGCCGACCACTACCAGCAGAACACCCCCATCGGCGACGGCCCCGTG
CTGCTGCCCGACAACCACTACCTGAGCACCCAGTCCGCCCTGAGCAAAGACCCCAACGAGAAGCGCGATCACATG
GTCCTGCTGGAGTTCGTGACCGCCGCCGGGATCACTCTCGGCATGGACGAGCTGTACAAGTAAGAAACCATTATG
AAGAAGAGAGTCCATATTTCAATTTCCAAGAGCTGAGGCAATTCTAACTTTTTTGCTATCCAGCTATTTTTATTT
GTTTGTGCATTTGGGGGGAATTCATCTCTCTTTAATATAAAGTTGGATGCGGAACCCAAATTACGTGTACTACAA
TTTAAAGCAAAGGAGTAGAAAGACAGAGCTGGGATGTTTCTGTCACATCAGCTCCACTTTCAGTGAAAGCATCAC
TTGGGATTAATATGGGGATGCAGCATTATGATGTGGGTCAAGGAATTAAGTTAGGGAATGGCACAGCCCAAAGAA
GGAAAAGGCAGGGAGCGAGGGAGAAGACTATATTGTACACACCTTATATTTACGTATGAGACGTTTATAGCCGAA
ATGATCTTTTCAAGTTAAATTTTATGCCTTTTATTTCTTAAACAAATGTATGATTACATCAAGGCTTCAAAAATA
CTCACATGGCTATGTTTTAGCCAGTGATGCTAAAGGTTGTATTGCATATATACATATATATATATATATATATAT
ATATATATATATATATATATATATATATATATATTTTAATTTGATAGTATTGTGCATAGAGCCACGTATGTTTTT
GTGTATTTGTTAATGGTTTGAATATAAACACTATATGGCAGTGTCTTTCCACCTTGGGTCCCAGGGAAGTTTTGT
GGAGGAGCTCAGGACACTAATACACCAGGTAGAACACAAGGTCATTTGCTAACTAGCTTGGAAACTGGATGAGGT
CATAGCAGTGCTTGATTGCGTGGAATTGTGCTGAGTTGGTGTTGACATGTGCTTTGGGGCTTTTACACCAGTTCC
TTTCAATGGTTTGCAAGGAAGCCACAGCTGGTGGTATCTGAGTTGACTTGACAGAACACTGTCTTGAAGACAATG
GCTTACTCCAGGAGACCCACAGGTATGACCTTCTAGGAAGCTCCAGTTCGATGGGCCCAATTCTTACAAACATGT
GGTTAATGCCATGGACAGAAGAAGGCAGCAGGTGGCAGAATGGGGTGCATGAAGGTTTCTGAAAATTAACACTGC
TTGTGTTTTTAACTCAATATTTTCCATGAAAATGCAACAACATGTATAATATTTTTAATTAAATAAAAATCTGTG
GTGGTCGTTTTATTATAGTTCCGGAGCTATATAGCTCCTATCATTCTATCATAACCTTAGAATACCAGAGAACAT
ATCATCTCATCTAATTATCTCTTACTATATGTGAAAAAAATGAAGGACATGGGGGAAGTGTGACTTGCCCCAAAT
CACATATTTCATGGTAGAGCCAGGTCTTCTGTTTGTCATATCAGTGTTCTTCCTGCCACAACCATCTTGAAGAAT
CTATTTCTCAGTAAGAAAATATCTTTATGGAGAGTAGCTGGAAAACAGTTGAGAGATGGAGGGGAGGCTGGGGGT
GTGGAGAGGGGAAGGGGTAAGTGATAGATTCGTTGAAGGGGGGAGAAAAGGCCGTGGGGATGAAGCTAGAAGGCA
GAAGGGCTTGCCTGGGCTTGGCCATGAAGGAGCATGAGTTCACTGAGTTCCCTAGGCATGTCTCATGCTAGCTCT
CGATGCTTTA

Nucleic acids according to the invention may comprise DNA or RNA. They may be single-stranded or double-stranded. They may also be polynucleotides which include within them synthetic or modified nucleotides. A number of different types of modification to oligonucleotides are known in the art These include methylphosphonate and phosphorothioate backbones, addition of acridine or polylysine chains at the 3′- and/or 5′-ends of the molecule. For the purposes of the use as described herein, it is to be understood that the polynucleotides may be modified by any method available in the art. Such modifications may be carried out in order to enhance the in vivo activity or life span of polynucleotides of interest.

### Vector

The present invention also provides a vector which comprises the nucleic acid construct or a nucleic acid sequence encoding a site-directed nuclease according to the present invention.

Such a vector may be used to introduce the nucleic acid sequence(s) into a host cell so that it expresses CTLA4.

The vector may be, for example, a plasmid or a viral vector, such as a retroviral vector or a lentiviral vector, or a transposon based vector or synthetic mRNA.

The vector may be capable of transfecting or transducing a cell, such as a T cell.

In one embodiment, the vector is an adeno-associated virus (AAV) vector. Preferably, the vector is AAV serotype 6.

### Cell

The present invention further provides a cell or population of cells produced according to the methods of the invention.

The present invention further provides a cell or population of cells comprising the nucleic acid construct or vector according to the invention.

Thus, the present invention provides a cell or population of cells comprising a nucleic acid sequence comprising one or more of exon 2, exon 3 and exon 4 of CTLA4, or a sequence with at least 70% identity to exon 2, exon 3 and/or exon 4 of the CTLA4 gene, within a target nucleotide sequence at the 3′-end of Intron 1 of endogenous CTLA4.

The present invention provides a cell or population of cells comprising a nucleic acid sequence comprising one or more of exon 2, exon 3 and exon 4 of CTLA4, or a sequence with at least 70% identity to exon 2, exon 3 and/or exon 4 of the CTLA4 gene, within a target nucleotide sequence, wherein the target sequence is within the sequence which corresponds to position 1268-2534, 1550-2400, 1900-2330 or 2097-2116 of SEQ ID No: 2.

The nucleic acid sequence comprising one or more of exon 2, exon 3 and exon 4 of CTLA4 may be any suitable sequence as described herein.

Suitably the target nucleotide sequence is within the sequence which corresponds to position 1900-2330 of SEQ ID No: 2.

Suitably the target nucleotide sequence is within the sequence which corresponds to position 2097-2116 of SEQ ID No: 2.

The cell according to the invention may express exogenous CTLA4.

The cell may comprise a mutation in the endogenous CTLA4 gene and/or be deficient in endogenous CTLA4 expression.

In one aspect, the cell according to the present invention expresses exogenous CTLA4 at normal physiological levels, or at supraphysiological levels, in comparison to a healthy control. Cells produced according to the methods of the present invention, or which comprise a nucleic acid construct according to the present invention, may be identified by determining CTLA4 surface expression and/or function.

Suitable methods for assaying CTLA4 function are known in the art. For example, CTLA4 function can be assessed using transendocytosis (TE) assays, which determine the ability of CTLA4 in a cell population to capture fluorescent-marked ligands from opposing cells. Edited cells are incubated with cells expressing fluorescently labelled CD80 or CD86. Uptake of ligand by the cells may then be assessed by flow cytometry.

In some embodiments, the repair template may comprise a reporter gene, for example, GFP. Accordingly, cells that are successfully edited express GFP and may be determined by fluorescence-activated cell sorting (FACS).

Alternatively, successful gene editing may be demonstrated by in-out PCR and Sanger sequencing of the edited locus. The primers GCTACTCCTGGGTGACAGAGG (SEQ ID No: 26) and TCATGTAGGTTGCCGCACAGA (SEQ ID No: 27) may be used to determine successful insertion of the replacement CTLA4 sequences in to the 3′-end of intron 1 of the endogenous CTLA4 gene.

The cell may be a CD3+ T lymphocyte or haematopoietic stem cell (HSC).

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ T_{reg} cells have been described—naturally occurring T_{reg} cells and adaptive T_{reg} cells. Naturally occurring T_{reg} cells (also known as CD4+CD25+FoxP3+ T_{reg} cells) arise in the thymus and have been linked to interactions between developing T cells with both myeloid (CD11c+) and plasmacytoid (CD123+) dendritic cells that have been activated with TSLP.

Naturally occurring T_{reg} cells can be distinguished from other T cells by the presence of an intracellular molecule called FoxP3. Mutations of the FOXP3 gene can prevent regulatory T cell development, causing the fatal autoimmune disease IPEX.

Adaptive T_{reg} cells (also known as Tr1 cells or Th3 cells) may originate during a normal immune response.

Preferably, the cell is a regulatory T cell.

Suitably, the cell may be a haematopoietic stem cell or haematopoietic progenitor cell. Suitably, the cell is an induced pluripotent stem cell (iPSC). Suitably, the cell may be obtained from umbilical cord blood. Suitably, the cell may be obtained from adult peripheral blood.

In some aspects, hematopoietic stem and progenitor cell (HSPCs) may be obtained from umbilical cord blood. Cord blood can be harvested according to techniques known in the art (e.g., U.S. Pat. Nos. 7,147,626 and 7,131,958, which are incorporated herein by reference).

In one aspect, HSPCs may be obtained from pluripotent stem cell sources, e.g., induced pluripotent stem cells (iPSCs) and embryonic stem cells (ESCs).

As used herein, the term “hematopoietic stem and progenitor cell” or “HSPC” refers to a cell which expresses the antigenic marker CD34 (CD34+) and populations of such cells. In particular embodiments, the term “HSPC” refers to a cell identified by the presence of the antigenic marker CD34 (CD34+) and the absence of lineage (lin) markers. The population of cells comprising CD34+ and/or Lin(−) cells includes haematopoietic stem cells and hematopoietic progenitor cells.

HSPCs can be obtained or isolated from bone marrow of adults, which includes femurs, hip, ribs, sternum, and other bones. Bone marrow aspirates containing HSPCs can be obtained or isolated directly from the hip using a needle and syringe. Other sources of HSPCs include umbilical cord blood, placental blood, mobilized peripheral blood, Wharton's jelly, placenta, fetal blood, fetal liver, or fetal spleen. In particular embodiments, harvesting a sufficient quantity of HSPCs for use in therapeutic applications may require mobilizing the stem and progenitor cells in the subject.

As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell (HSC and HPC respectively).

As used herein, the term “reprogramming” refers to a method of increasing the potency of a cell to a less differentiated state.

As used herein, the term “programming” refers to a method of decreasing the potency of a cell or differentiating the cell to a more differentiated state.

The cells of the invention may be any of the cell types mentioned above.

The cell may be autologous or allogenic.

For example, cells according to the invention may either be created ex vivo either from a patient's own peripheral blood, or in the setting of a haematopoietic stem cell transplant from donor peripheral blood, or peripheral blood from an unconnected donor.

Alternatively, cells may be derived from ex vivo differentiation of inducible progenitor cells or embryonic progenitor cells to, for example, T cells. Alternatively, an immortalized T cell line may be used.

The cell of the invention may be an ex vivo cell from a subject. The cell may be from a peripheral blood mononuclear cell (PBMC) sample. The cells may be activated and/or expanded prior to introduction of the site-directed nuclease and nucleic acid construct of the invention, for example by treatment with an anti-CD3 monoclonal antibody.

T cells may be isolated using methods which are well known in the art. For example, T cells may be purified from single cell suspensions generated from samples on the basis of expression of CD3, CD4 or CD8. T cells may be enriched from samples by passage through a Ficoll-paque gradient.

The cell of the invention may be made by:
i) isolating a cell-containing sample from a subject or other sources listed above; and
ii) introducing a nucleic acid sequence or vector, and a site-directed nuclease according to the present invention into the cells.

The cells may then by purified, for example, selected on the basis of expression of CTLA4 or a reporter gene.

### Kit

The present invention also provides a kit of polynucleotides or a kit of vectors.

The kit may comprise:
i) a polynucleotide comprising the gRNA according to the invention;
ii) a polynucleotide comprising the nucleic acid construct according to the invention;
iii) optionally, a polynucleotide encoding a Cas protein.

The kit may comprise:
i) a vector which comprises the gRNA according to the invention;
ii) a vector which comprises the nucleic acid construct according to the invention;
iii) optionally, a vector which comprises a polynucleotide encoding a Cas protein.

### Pharmaceutical Composition

The present invention also relates to a pharmaceutical composition containing a cell or plurality of cells of the present invention. In particular, the invention relates to a pharmaceutical composition containing a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating and/or preventing a disease which comprises the step of administering a cell of the present invention (for example in a pharmaceutical composition as described above) to a subject.

Suitably, the present methods for treating and/or preventing a disease may comprise administering a cell of the invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
i) isolation of a cell-containing sample;
ii) introducing into the cells a nucleic acid construct or vector according to the invention and a site-directed nuclease according to the invention;
iii) administering the cells from (ii) to a subject.

Preferably, the subject is a mammal, preferably a human, cat, dog, horse, donkey, sheep, pig, goat, cow, mouse, rat, rabbit or guinea pig, but most preferably the subject is a human.

The present invention provides a cell of the present invention for use in treating and/or preventing a disease.

The invention also relates to the use of a cell in the manufacture of a medicament for the treatment and/or prevention of a disease.

The disease to be treated and/or prevented by the method of the present invention may be an immune deficiency disease.

The immune deficiency disease may be common variable immune deficiency (CVID), hypogammaglobulinemia, recurrent infections, granulomatous lymphocytic interstital lung disease (GLILD), fibrosis, and/or bronchiectasis.

The disease to be treated and/or prevented by the method of the present invention may be an autoimmune disease.

The autoimmune disease may be type 1 diabetes, autoimmune thyroiditis, arthritis, rheumatoid arthritis, juvenile idiopathic arthritis, psoriasis, psoriatic arthritis, lupus, uveitis, vitiligo, myasthenia gravis, immune thrombocytopenia, enteropathy, autoimmune hemolytic anemia, and/or autoimmune neutropenia.

The disease to be treated and/or prevented by the method of the present invention may be cancer.

The cancer may be such as bladder cancer, breast cancer, colon cancer, endometrial cancer, kidney cancer (renal cell), leukaemia, lung cancer, melanoma, pancreatic cancer, prostate cancer and thyroid cancer.

The cancer may be acute lymphoblastic leukemia (ALL), acute myelogenous leukemia (AML), chronic lymphocytic leukemia (CLL), chronic myelogenous leukemia (CML), acute monocytic leukemia (AMoL), Hodgkin's lymphoma and/or non-Hodgkin's lymphoma.

The disease to be treated and/or prevented by the method of the present invention may be associated with solid organ and/or haematopoietic stem cell transplantation, for example transplant rejection and/or graft-versus-host disease (GvHD).

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

### Aspects of the Invention

Aspects of the invention are provided in the following numbered paragraphs (paras):
1. A method of engineering a cell, comprising the steps of introducing into the cell:
   i) a site-directed nuclease which is capable of cleaving a target nucleotide sequence at the 3′-end of Intron 1 of a Cytotoxic T-Lymphocyte Associated Protein 4 gene (CTLA4); and
   ii) a nucleic acid construct which comprises a nucleic acid sequence comprising one or more of exon 2, exon 3 and exon 4 of CTLA4, or a sequence with at least 70% identity to exon 2, exon 3 and/or exon 4 of the CTLA4 gene, and 5′- and 3′-homology arms, wherein each of the 5′ and 3′ homology arms is essentially complementary to a sequence flanking the target nucleotide sequence at the 3′-end of Intron 1 of CTLA4.
2. The method according to para 1, wherein the target nucleotide sequence is within the sequence which corresponds to position 1268-2534, 1550-2400, 1900-2330 or 2097-2116 of SEQ ID No: 2.
3. The method according to para 1 or para 2 wherein the nuclease comprises a CRISPR-associated protein (Cas) and a gRNA molecule, wherein the gRNA is complementary to the target nucleotide sequence at the 3′-end of Intron 1 of CTLA4, preferably wherein the CRISPR-associated protein is Cas9.
4. The method according to para 1 to 3, wherein the nuclease is a CRISPR-associated protein (Cas) in complex with a gRNA molecule, wherein the gRNA is complementary to the target nucleotide sequence at the 3′-end of Intron 1 of CTLA4.
5. The method according to para 4, wherein the gRNA comprises any one of SEQ ID Nos: 11, 14 or 17.
6. The method according to para 5, wherein the gRNA comprises SEQ ID No: 11.
7. The method according to any of paras 3 to 6, wherein the CRISPR-associated protein is Cas9.
8. The method according to any of paras 1 to 7, wherein the nucleic acid construct further comprises a reporter gene.
9. The method according to any of paras 1 to 8, wherein the nucleic acid construct is introduced into the cell in a vector, preferably wherein the vector is an adeno-associated virus (AAV) vector.
10. The method according to any of paras 1 to 9, wherein the cell is a haematopoietic stem cell (HSC).
11. The method according to any of paras 1 to 9, wherein the cell is a CD3+ T cell.
12. The method according to para 11, wherein the cell is a regulatory T cell.
13. The method according to any of paras 1 to 12, wherein the cell is isolated from a subject.
14. The method according to any of paras 1 to 13, wherein the cell comprises a mutation in endogenous CTLA4 and/or is deficient in endogenous CTLA4 expression.
15. A site-directed nuclease which is capable of cleaving a target nucleotide sequence at the 3′-end of Intron 1 of CTLA4.
16. The site-directed nuclease according to para 15, wherein the target nucleotide sequence is within the sequence which corresponds to position 1268-2534, 1550-2400, 1900-2330 or 2097-2116 of SEQ ID No: 2.
17. The site-directed nuclease according to para 15 or para 16, wherein the nuclease comprises a CRISPR-associated protein and a gRNA molecule, wherein the gRNA is complementary to a nucleotide sequence at the 3′-end of Intron 1 of CTLA4.
18. The site-directed nuclease according to para 15 or para 16, wherein the nuclease is a CRISPR-associated protein in complex with a gRNA molecule, wherein the gRNA is complementary to a nucleotide sequence at the 3′-end of Intron 1 of CTLA4.
19. The site-directed nuclease according to para 17 or 18, wherein the gRNA comprises any one of SEQ ID Nos: 11, 14 or 17.
20. The site-directed nuclease according to para 19, wherein the gRNA comprises SEQ ID No: 11.
21. The site-directed nuclease according to any of paras 17 to 20, wherein the CRISPR-associated protein is Cas9.
22. A gRNA comprising any of SEQ ID Nos: 11, 14 or 17.
23. A vector comprising the gRNA of para 22, preferably wherein the vector is an adeno-associated virus (AAV) vector.
24. A nucleic acid construct which comprises a nucleic acid sequence comprising one or more of exon 2, exon 3 and exon 4 of the CTLA4 gene, or a sequence with at least 70% identity to exon 2, exon 3 and/or exon 4 of the CTLA4 gene, and 5′- and 3′-homology arms, wherein each of the homology arms is essentially complementary to a sequence flanking a target nucleotide sequence at the 3′-end of Intron 1 of CTLA4.
25. The nucleic acid construct according to para 24, wherein the target nucleotide sequence is within the sequence which corresponds to position 1268-2534, 1550-2400, 1900-2330 or 2097-2116 of SEQ ID No: 2.
26. The nucleic acid construct according to para 24 or para 25, which further comprises a reporter gene.
27. A vector comprising the nucleic acid construct according to any of paras 24 to 26, preferably wherein the vector is an adeno-associated virus (AAV) vector.
28. A kit of polynucleotides comprising:
   i) a polynucleotide comprising the gRNA according to para 22,
   ii) a polynucleotide comprising the nucleic acid construct according to any of para 24 to 26, and
   iii) optionally, a polynucleotide encoding a Cas protein.
29. A kit of vectors comprising:
   i) a vector according to para 23.
   ii) a vector according to para 24.
30. A cell comprising the nucleic acid construct according to any of paras 24 to 26.
31. A cell engineered according to the method of any one of paras 1 to 14.
32. The cell according to para 30 or 31, wherein the cell is a haematopoietic stem cell (HSC).
33. The cell according to para 30 or 31, wherein the cell is a CD3+ T cell.
34. The cell according to para 33, wherein the cell is a regulatory T cell.
35. The cell according to any of paras 30 to 34, wherein the cell is isolated from a subject.
36. The cell according to any of paras 30 to 35, wherein the cell comprises a mutation in endogenous CTLA4 and/or is deficient in endogenous CTLA4 expression.
37. A pharmaceutical composition comprising the cell according to any of paras 30 to 36.
38. A method of treating and/or preventing a disease in a subject comprising administering the cell according to any of paras 30 to 36.
39. A method according to para 38, which comprises the following steps:
   i) isolation of a cell-containing sample from a subject;
   ii) introducing into the cells a nucleic acid construct according to any of paras 24 to 26 or a vector according to para 27 and a site-directed nuclease according to any of paras 15 to 21; and
   iii) administering the cells from (ii) to a subject.
40. Use of a cell according to any of paras 30 to 36 in the manufacture of a medicament for the treatment and/or prevention of a disease.
41. A cell according to any of paras 30 to 36, for use in treating and/or preventing disease in a subject.
42. The method according to para 38 or 39, use according to para 40, or cell for use according to para 41, wherein the disease is an immune deficiency disease.
43. The method, use, or cell for use according to para 42, wherein the disease is common variable immune deficiency (CVID), hypogammaglobulinemia, recurrent infections, granulomatous lymphocytic interstital lung disease (GLILD), fibrosis, and/or bronchiectasis.
44. The method according to para 38 or 39, use according to para 40, or cell for use according to para 41, wherein the disease is an autoimmune disease.
45. The method, use, or cell for use according to para 44, wherein the disease is type 1 diabetes, autoimmune thyroiditis, arthritis, rheumatoid arthritis, juvenile idiopathic arthritis, psoriasis, psoriatic arthritis, lupus, uveitis, vitiligo, myasthenia gravis, immune thrombocytopenia, enteropathy, autoimmune hemolytic anemia, and/or autoimmune neutropenia.
46. The method according to para 38 or 39, use according to para 40, or cell for use according to para 41, wherein the disease is cancer.
47. The method, use, or cell for use according to para 46, wherein the cancer is acute lymphoblastic leukemia (ALL), acute myelogenous leukemia (AML), chronic lymphocytic leukemia (CLL), chronic myelogenous leukemia (CML), acute monocytic leukemia (AMOL), Hodgkin's lymphoma and/or non-Hodgkin's lymphoma.
48. The method according to para 38 or 9, use according to para 40, or cell for use according to para 41, wherein the disease is associated with solid organ and/or haematopoietic stem cell transplantation, for example transplant rejection and/or graft-versus-host disease (GvHD).

The invention will now be further described by way of Examples, which are meant to serve to assist one of ordinary skill in the art in carrying out the invention and are not intended in any way to limit the scope of the invention.

### EXAMPLES

### Example 1—Cell Isolation and Culture

Peripheral blood mononuclear cells (PBMCs) were isolated from patients with CTLA4-deficiency and healthy controls.

T cells were isolated by MACS using the pan T cell or CD4+ T Cell Isolation Kit (Miltenyi Biotec). T cells were activated using Transact CD3/CD28 beads (Miltenyi Biotec 130-111-160) at a titre of 1:100.

CD3 or CD4 selected human cells were cultured in TexMACS media (Miltenyi Biotec, 130-097-196) supplemented with 1% Penicillin/Streptomycin (100 U/ml; GIBCO, 15070), IL-2 human (100 U/ml; Roche 11147528001), IL-7 human (BD, 554608) and IL-15 human (BD, G243-886). T cells were activated using Transact CD3/CD28 beads (Miltenyi Biotec 130-111-160) at a titre of 1:100.

### Example 2—rAAV6 Donor Manufacture and Production

Donor templates were designed using snapgene (Snapgene, USA) software. Sequences for the insert were manufactured by Geneart™ (Thermofisher, USA) with restriction sites Pacl and AvRII at the 5′- and 3′-ends respectively. This insert was then cloned into a rAAV6 vector. The correct sequence was confirmed with sanger sequencing. rAAV vectors were produced with a standard double transfection method that introduces an ITR-containing transfer plasmid and a single helper plasmid, pDGM6 which contains the AAV2 rep and AAV6 cap proteins and the adenoviral proteins and RNA required for helper functions. Vector production took place in HEK293T cells seeded at 15×10⁶ in 15×15 cm dish per construct. 24 μg of pDGM6 (per plate) and 12 μg of ITR-containing plasmid were transfected to each 15 cm dish and branched polyethylenimine added at a 4:1 ratio to DNA. Complete DMEM media (Life Tech, USA) was changed 4 hours after transfection. 24 hours after transfection the media was changed again but replaced this time by DMEM with 2% FCS and 1% penicillin/streptomycin. A further 48 hours later, supernatant was harvested and treated with ammonium sulphate (Sigma-Aldrich, UK) (31.3 g per 100 ml supernatant), pelleted (centrifuge at 8300×g for 30 minutes) and re-suspended in 10 ml total volume of 1×TD buffer (diluted from 5×TD: 5×PBS, 5 mM MgCl₂, 12.5 mM KCl). This solution was then treated with 50 U/ml Benzonase (Sigma-Aldrich, UK) and incubated at 37° C. for 30 minutes and stored at 4° C. for no more than 24 hours before purification. Simultaneously, cells were harvested using cell scrapers, pelleted (centrifuge 1400×g for 10 minutes), washed in PBS and resuspended in 10 ml 1×TD buffer. Three freeze-thaw cycles were performed to harvest AAV6 from the cell pellet, 0.5% deoxycholic acid sodium salt (VWR, USA) and benzonase 50 U/ml added and the solution incubated for 30 minutes at 37° C. The lysate was pelleted (4000×g for 30 minutes at 18° C.) and supernatant removed and stored at 4° C. prior to purification. The two solutions were combined and AAV6 vectors purified by lodixanol density gradient and ultra-centrifugation at 40,000 rpm (273,799×g) with no brake for 2 hours at 18° C. AAV6 particles were extracted using a needle and syringe between the 40% and 60% gradient interface and dialysed 3 times in 1×PBS (ThermoFisher, USA) with 5% sorbitol (Sigma-Aldrich, UK) in the third step using 10K MWCO Slide-a-Lyzer dialysis cassettes (ThermoFisher, USA). Titration was performed using Quick Titre AAV Quantification Kit (Cell Biolabs, USA) prior to aliquoting and storage at −80° C. before use.

### Example 3—Electroporation and Transduction

HiFi Cas9 protein was purchased from Integrated DNA technologies (IDT, USA) and synthetic gRNAs were custom-made by Synthego (Synthego, USA). Cas9 and gRNA were mixed at a 1:3 molar ratio and incubated at 25° C. for 30 minutes to form RNPs. A Lonza Nucleofector 4D was used for nucleofection (programme EO-115) with a P3 Primary Cell 4D-Nucleofector Kit (Lonza, V4XP-3032). 1×10⁶ CD4+ or T_{reg} cells per reaction were washed twice in PBS and resuspended in 15 μl/per reaction of P3 nucleofector solution. Cells were mixed 1:1 with their respective RNP solution (30 μl total volume) and transferred to the nucleofector strip. Immediately following nucleofection, 80 μl of warmed TexMACs (Milltenyi biotech, Germany) media was added to the cells and then 80 μl was transferred from the nucleofector strip to a 24 well plate containing artificial antigen presenting cells in 920 μl of warmed TexMACs media with IL-2 human (100 U/ml; Roche 11147528001), IL-7 human (BD, 554608) and IL-15 human (BD, G243-886) for CD4+ cells and IL-2 (100 units/μl) and aCD3 (100 ng/ml) for isolated T_{regs}. AAV6 was added at 13,000 MOI (vector genomes/cell) within 15 minutes of nucleofection and incubated for 24 hours. After 24 hours cell density was adjusted to 0.5×10⁶/ml using TexMACs media (with IL-2 100 units/μl). Cells were phenotyped >48 hours post editing by FACS to assess editing efficiency.

### Example 4—Targeted Insertion in to the 5′-UTR of Exon 1

Candidate gRNAs underwent in silico assessment of on-target and off-target activity and the four top scoring gRNAs were assessed in vitro. The target TGGCTTGCCTTGGATTTCAG (SEQ ID No: 28; gRNA: UGGCUUGCCUUGGAUUUCAG, SEQ ID No: 29) produced on-target indels in 91% of cells when analysed by ICE (inference of CRISPR edits) and resulted in almost complete knock out of CTLA4 expression on assessment by flow cytometry.

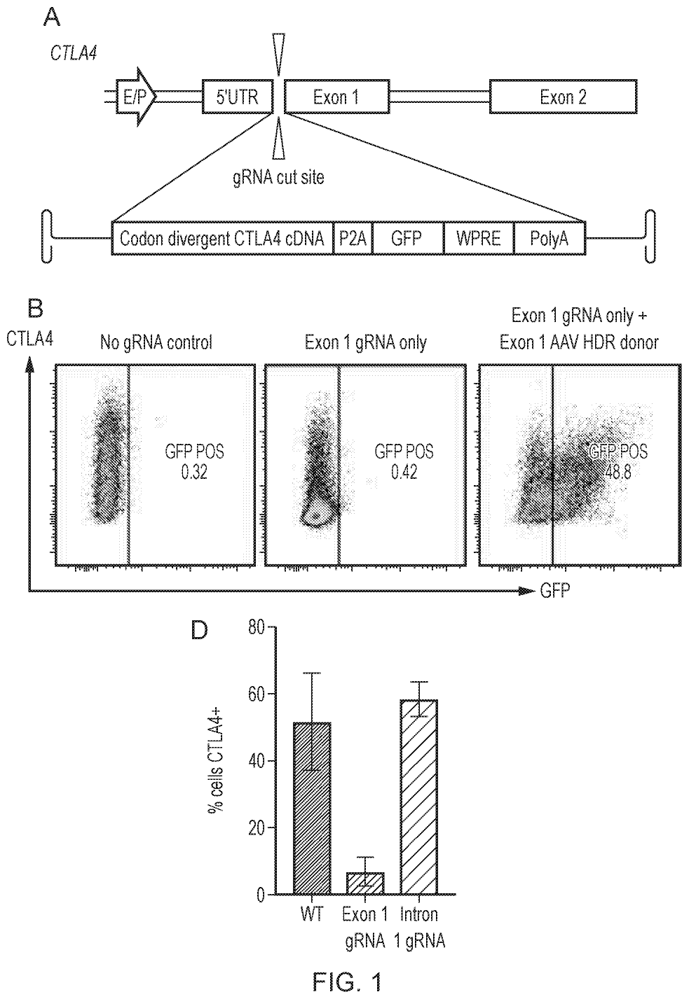

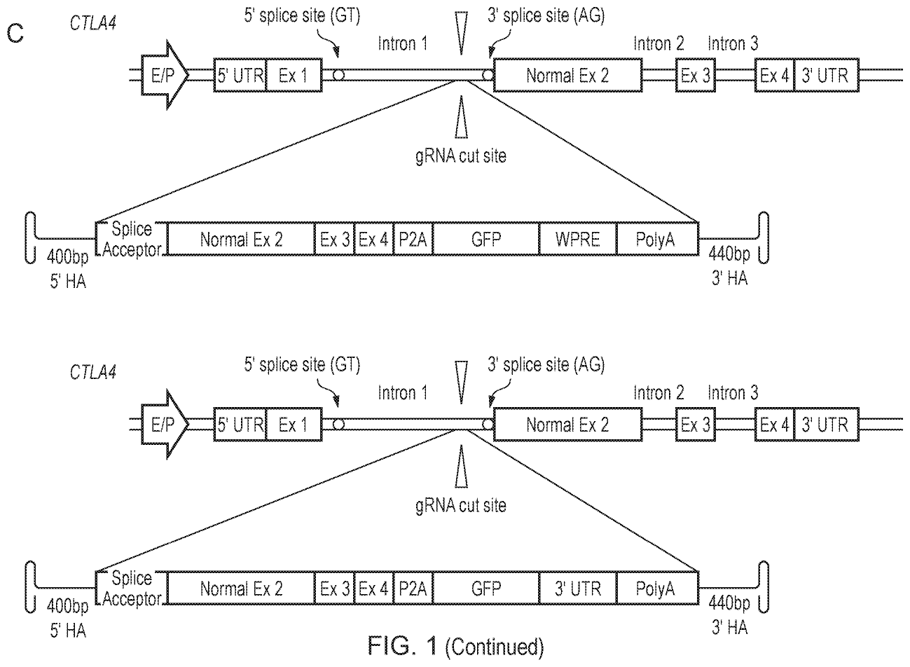

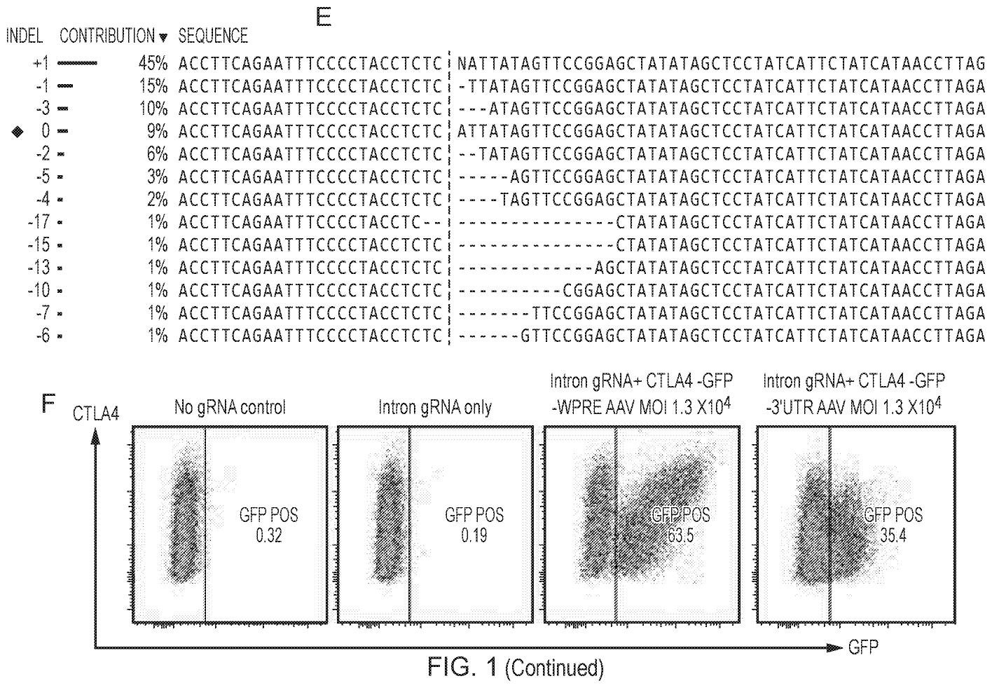

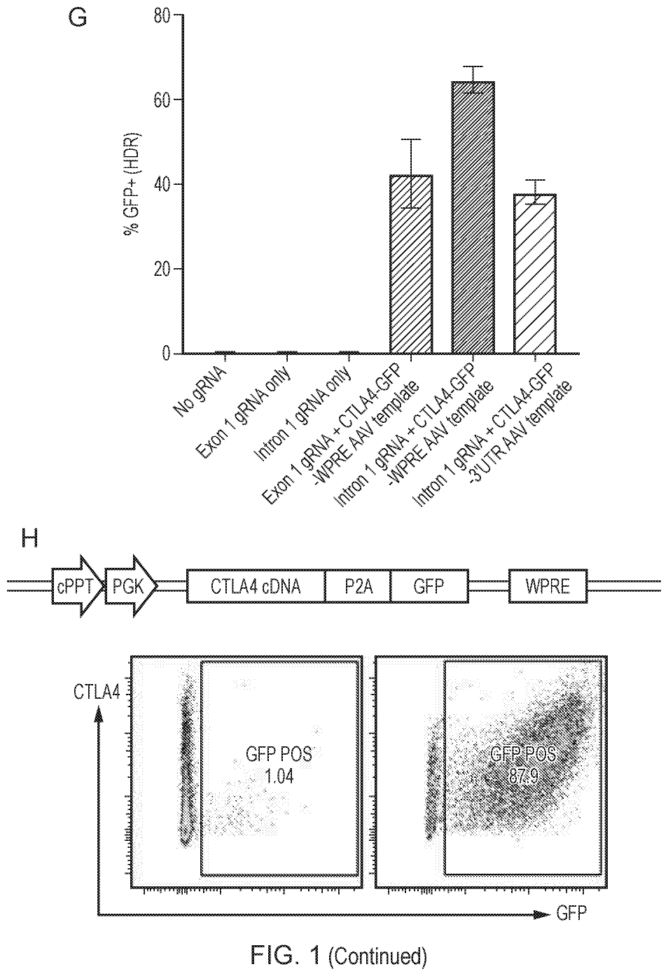

An rAAV HDR template was designed to insert a corrective cDNA template followed by a P2A sequence, GFP reporter cassette and WPRE sequence flanked by two asymmetrical homology arms (FIG. 1A).

This gRNA/rAAV editing strategy was assessed in wild type human T cells. After editing HDR was assessed by evaluating CTLA4 and GFP expression by flow cytometry (FIG. 1B). Editing efficiencies of >40% were reproducibly achieved using this approach in CD3+ T cells from healthy donors as measured by the percentage of GFP+ cells.

### Example 5—Targeted Insertion in to 3′-end of Intron 1

CRISPR guide RNAs (gRNAs) were designed using the Benchling online tool (https://www.benchling.com/crispr/). NGG PAM sequences were identified towards the 3′-end of the first intron of CTLA4 and assessed in silico for on-target and off-target activity using the Benchling online tool. The top three scoring gRNAs were ordered as synthetic gRNAs from Synthego (Synthego, USA) and assessed in vitro in primary human T cells (AGCUCCGGAACUAUAAUGAG, SEQ ID No: 11; GAGAAAUAGAUUCUUCAAGA, SEQ ID No: 14 and GAUAUGACAAACAGAAGACC, SEQ ID No: 17).

48 hours following nucleofection, DNA was extracted using QuickExtract™ (Cambio) as per product protocol and then amplified by PCR using primers to create an 800 bp amplicon that incorporated the cut-site. The PCR product was purified using MonarchR PCR & DNA clean-up kit (New England Biolabs, T1030S) and both control and edited samples were sent for sanger Sequencing. Sequencing results were then analysed using the Synthego ICE software (ice.synthego.com).

The gRNAs GAGAAAUAGAUUCUUCAAGA (SEQ ID No: 14) and GAUAUGACAAACAGAAGACC (SEQ ID No: 17) produced indels in approximately 50% and 60% cells, respectively. The gRNA sequence AGCUCCGGAACUAUAAUGAG (SEQ ID No: 11) reliably produced indels in >85% of cells as assessed by ICE (FIG. 1E) and was selected for the editing approach.

All three gRNAs had no effect on protein expression as assessed by flow cytometry, validating the hypothesis that a dsDNA break could be created in the intronic sequence without interrupting protein expression (FIG. 1D).

The gRNA AGCUCCGGAACUAUAAUGAG (SEQ ID No: 11) had no off-target activity, as assessed by guide SEQ analysis. Genome wide, off-target cleavage activities of the gRNA were assessed using capture of a short double-stranded oligonucleotide at double strand breaks (DSBs) through GUIDE-seq (genome wide, unbiased identification of DSBs enabled by sequencing). In brief, blunt double-stranded oligodeoxynucleotide (dsODNs) that have been phopsphothiorated on two of the phosphate linkages on the 5′ end of both strands were provided. These dsODNs preferentially integrate at any dsDNA break i.e. both on-target and off-target breaks. Human CD4+ T cells were sorted, activated and cultured, as described above. Nucleofection of the gRNA/Cas9 RNP was performed as above, with the dsODNs added to the nucleofection buffer. Cells were then cultured for 5 further days before being lysed and the genomic DNA extracted, purified, resuspended in 1×TE buffer. For analysis the extracted gDNA was sheared by sonication and the resulting sheared DNA underwent end-repair and adapter ligation. The DNA containing the dsODN insert was amplified via two rounds of PCR using primers complimentary to the dsODN. Next generation sequencing was used to identify the sequences flanking the dsODN cassette inserts.

An AAV6 HDR donor template was designed incorporating an artificial splice acceptor (SA) sequence, cDNA for exons 2, 3 and 4, P2A sequence, GFP reporter cassette and WPRE sequence flanked by two asymmetrical homology arms (FIG. 1C, top) (SEQ ID No: 20).

A second HDR donor was designed for this gRNA that swapped the WPRE sequence for the CTLA4 3′UTR in order to assess the effects of the WPRE sequence versus the 3′UTR sequence on mRNA transport, stability and thus gene expression (FIG. 1C, bottom) (SEQ ID No: 21). In contrast to the approach targeting the first exon, the intronic approach resulted in much higher editing efficiencies (reproducibly >65%) as measured by percentage of GFP+ cells by flow cytometry. Higher editing rates were observed using the WPRE containing HDR repair template compared to the 3′UTR construct, although the disparity in editing rates may be in-part due to the difference in size between the editing cassettes (FIGS. 1F and 1G).

### Example 6—Lentiviral Gene Addition

A lentiviral vector encoding CTLA4 cDNA followed by a P2A-GFP sequence under the influence of a phosphoglycerate kinase (PGK) promoter was designed (FIG. 1H, top). Wild type activated human T cells were transduced (at 80-90% efficiency) with this lentiviral vector (FIG. 1H, bottom).

### Example 7—Comparison of Gene Editing and Gene Addition Approaches on CTLA4 Function in Wild Type CD4+ T Cells

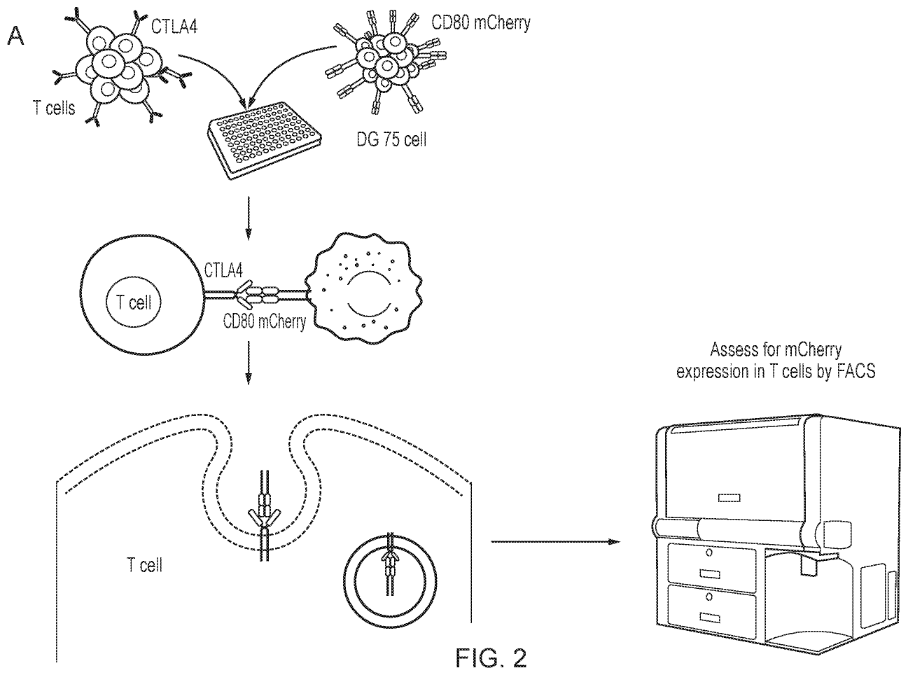

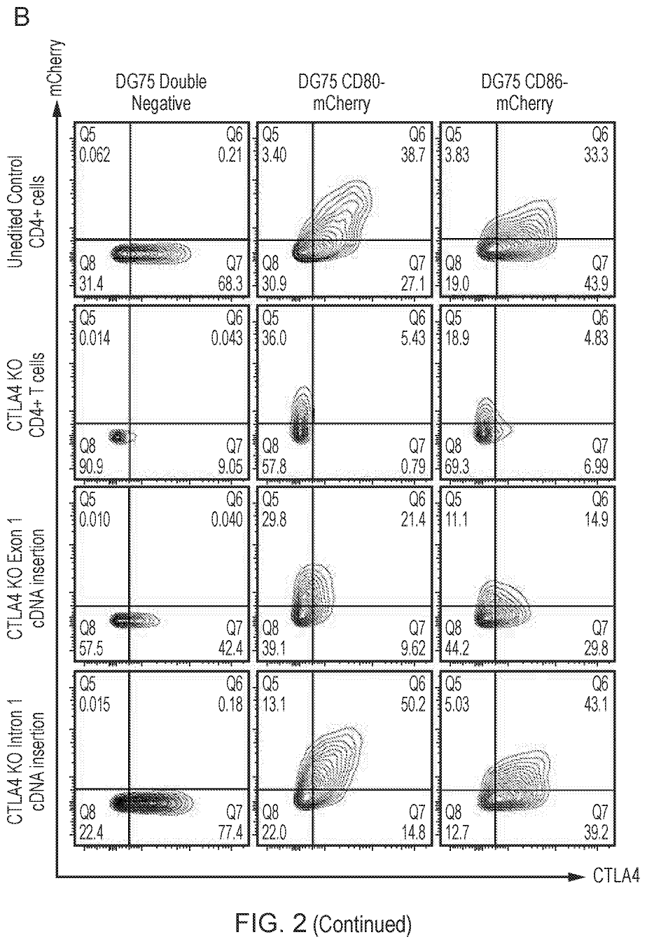

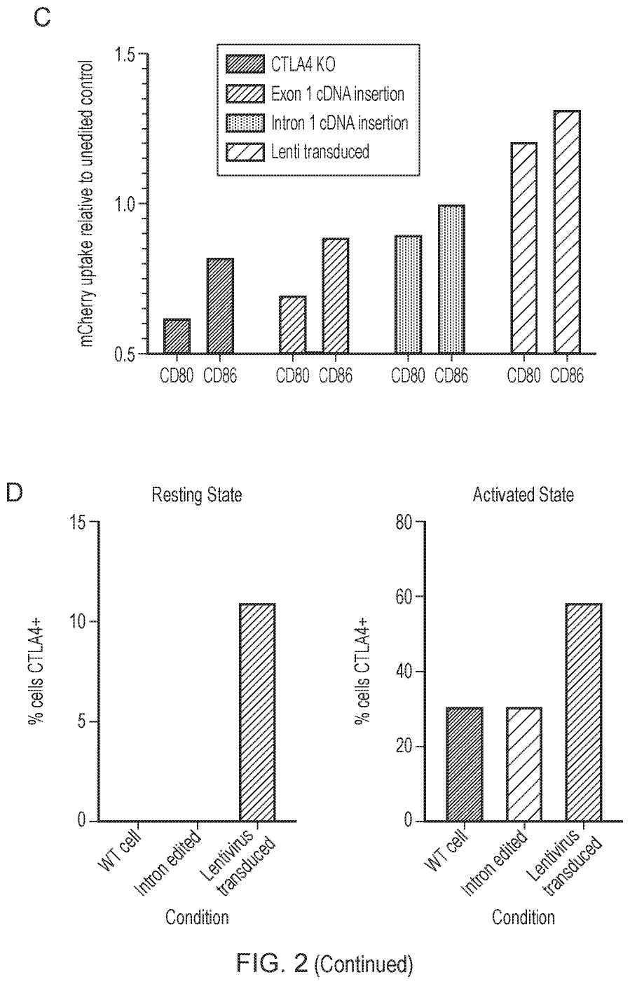

Whilst all three editing approaches (Exon 1, Intron 1—WPRE-donor, Intron 1—3′UTR donor) demonstrated high efficiencies in healthy T cells, the efficacy of these editing approaches and lentivirus transduction on CTLA4 function was also assessed. CTLA4 function can be assessed using TE assays which determine the ability of CTLA4 in a cell population to capture fluorescent-marked ligands (CD80 and CD86) from opposing cells (FIG. 2A).

Unedited healthy donor CD4+ cells were compared to cells that were edited with the exon 1 gRNA alone (resulting in knock down of CTLA4), the exon 1 cDNA repair strategy or the intronic editing strategy with the superior WPRE-containing HDR donor template. Five days after editing cells were put into an overnight TE assay with DG75 cells at a 5:1 ratio (T cells:DG75s) expressing either no ligand (double negative control), CD80-mCherry or CD86-mCherry. Ligand uptake (mCherry uptake into CD4+ T cells) was assessed 20 hours later by flow cytometry (FIG. 2B). As expected, knock down of CTLA4 almost entirely abolished CTLA4 mediated transendocytosis compared to the unedited control. Transendocytosis was restored to some degree in cells edited with the cDNA exon 1 insertion although the amount of ligand was nearly two-fold less than in the unedited cells. The cells edited with the intron 1 approach using the WPRE donor in comparison were able to transendocytose ligand in comparable amounts to the unedited cells further demonstrating the superiority of this approach (FIGS. 2B and 2C).

In a separate experiment, TE was assessed in healthy control T cells and healthy T cells which had been transduced with the CTLA4-P2A-GFP-WPRE lentivirus vector. High transduction efficiencies were obtained (>80%) using this vector (determined by GFP+ cells). As expected, the transduction of cells with the lentivirus vector resulted in supraphysiological levels of CTLA4 compared to untransduced healthy control T cells. CD4+ T cells transduced with the lentivirus vector also demonstrated increased TE of CD80 and CD86 compared to untransduced cells (FIG. 2C).

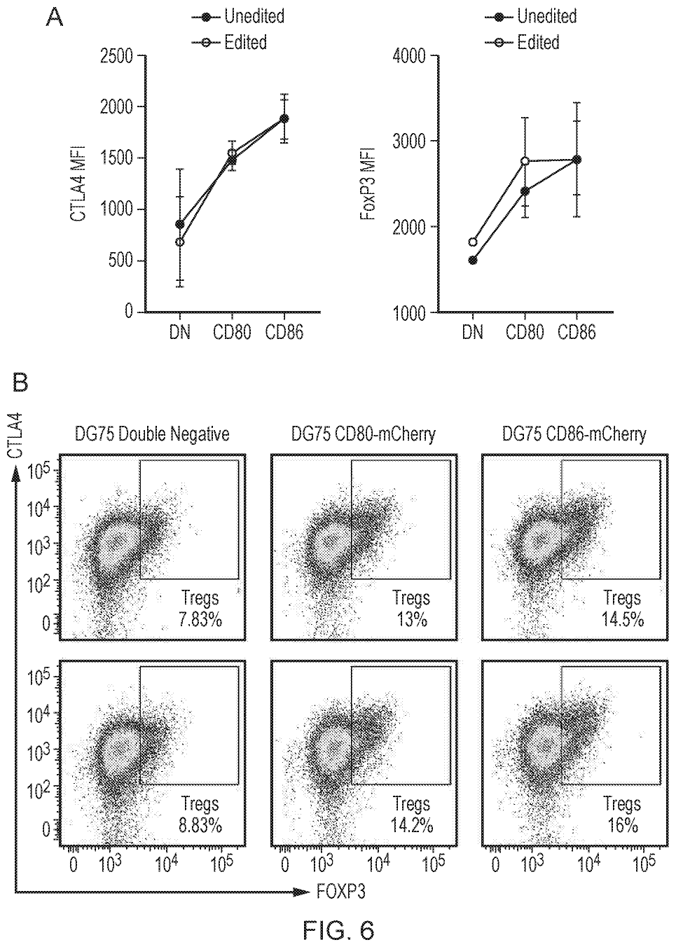

CD4+ T cells with edited CTLA4 retain normal functional characteristics To determine the functional characteristics of the edited T cells, we assessed the impact of gene editing on Treg survival. Since Treg require CD28 signalling for their homeostasis, we incubated edited and unedited Treg with DG75 B cells expressing either CD80, CD86 or no ligand. This also assessed the impact of CTLA4 expression since CTLA4 competes with CD28 for ligand binding in this system. Edited T cells were flow cytometrically sorted for GFP and compared with mock edited cells. After 5 days, both unedited and edited CD4+ T cells possessed a robust population of FoxP3+ Tregs following stimulation in the presence of CD80 or CD86, indicating that edited cells behaved indistinguishably from unedited cells (FIG. 6B). In addition, we observed that in both unedited and edited cells CD86-CD28 costimulation enhanced the expression of CTLA4 compared to CD80-CD28 costimulation (FIG. 6A) in line with previous observations. Together, this data indicated that Treg homeostasis, which integrates both CD28 and CTLA-4 function was normal in our edited cells.

### Example 8—Intronic Gene Editing Offers Advantages in Restoring Physiological Kinetics of Surface CTLA4 Expression Compared to Lentivirus Gene Addition

In order to assess the physiological kinetics of CTLA4 surface expression following gene editing or lentiviral transduction in conventional T cells, transduced/edited and untransduced/unedited cells were either rested or re-stimulated with CD3/CD28 beads and surface CTLA4 expression assessed by flow cytometry.

Following transduction of the CTLA4-P2A-GFP lentiviral vector, surface expression of CTLA4 was significantly increased compared to untransduced conventional T cells. Whilst resting cells transduced with the lentiviral vector had reduced CTLA4 expression compared to re-stimulated transduced cells, the surface expression of CTLA4 at rest was significantly increased compared to un-transduced controls (FIG. 2D). This demonstrates supraphysiological expression of CTLA4 that remains outside the normal regulatory machinery following lentiviral transduction (FIG. 2D). In contrast, healthy control conventional T cells which underwent gene editing with the intron 1 approach (WPRE-containing donor) demonstrated expression kinetics which mirrored those observed in unedited healthy cells (very low-level surface CTLA4 expression at rest with upregulation following stimulation) (FIG. 2D).

### Example 9—Intronic Gene Editing Restores CTLA4 Expression and Function in CD4+ T Cells With Heterozygous CTLA4 Mutations From Clinically Affected Patients

The previous experiments demonstrate that the intronic approach using the rAVV template encoding CTLA4 exons 2, 3 and 4—P2A-GFP—WPRE sequence resulted in the highest efficiency of editing. The functional profile and expression kinetics of cells edited using this approach mirrored those of healthy unedited cells. This approach was chosen to demonstrate proof-of-principle in cells isolated from patients with symptomatic CTLA4 deficiency. Due to the mutational landscape of CTLA4 deficiency with the majority of disease-causing mutations occurring in exons 2 and 3 this approach could be used in >80% of patients.

Peripheral blood mononuclear cells (PBMCs) were obtained from symptomatic patients with CTLA4 deficiency. CD4+ T cells were selected (MACSs sort) in order to maximise the number of edited T_{regs} from the limited patient samples that were available. Following editing of CD4+ cells with the intronic gRNA, rAAV donor, cells were assessed by flow cytometry for gene editing efficiency and CTLA4 surface expression. For comparison, a healthy control sample underwent a mock nucleofection and was kept in the same culture conditions as the patient samples and flow cytometry and functional assays performed at the same time points. As experiments were performed at separate time points due to sample collection, function was assessed as relative to the healthy control in order to mitigate slight differences in culture conditions and activation state that might occur between experiments separated in time.

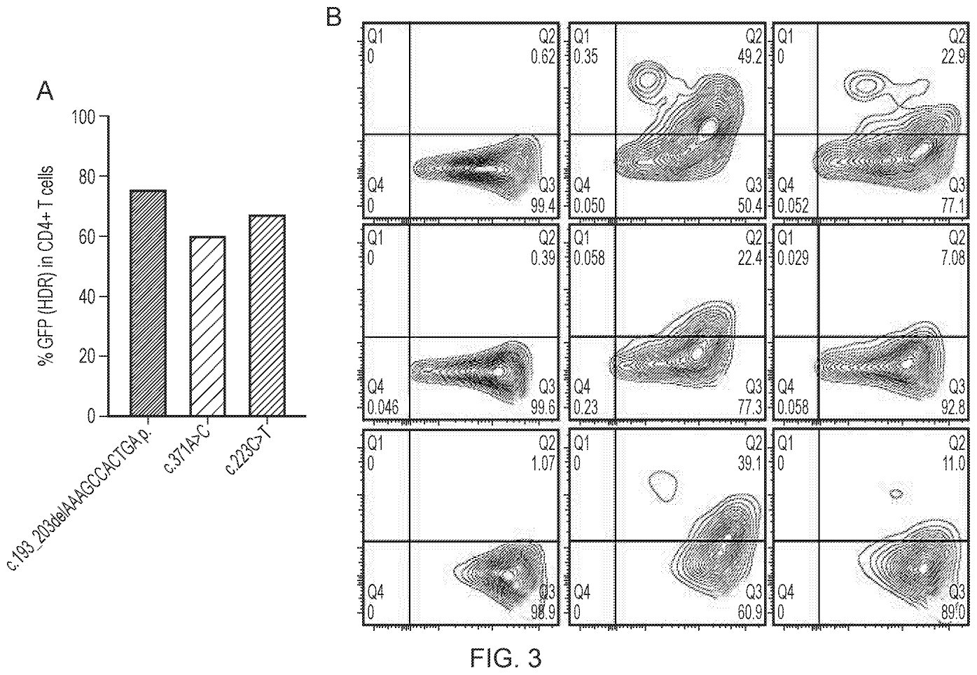

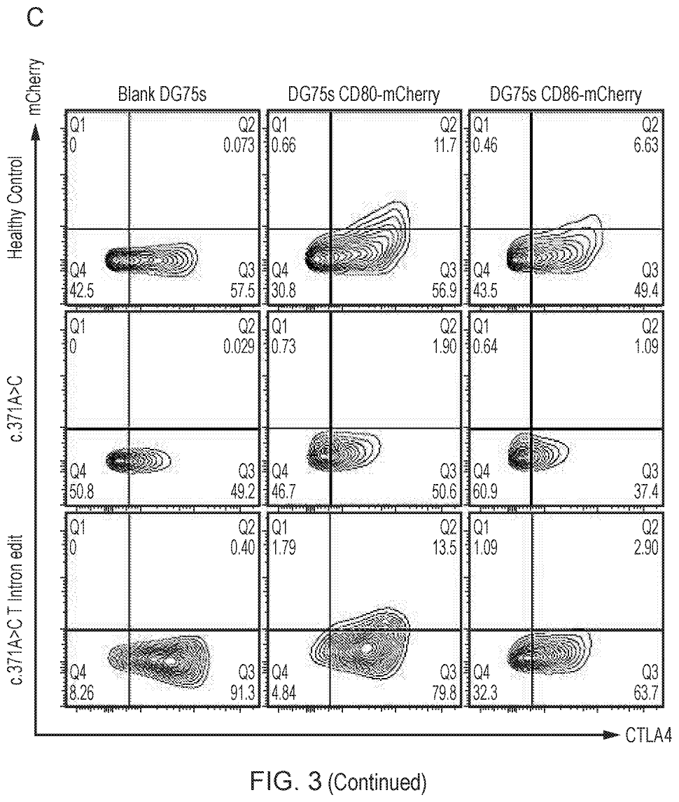

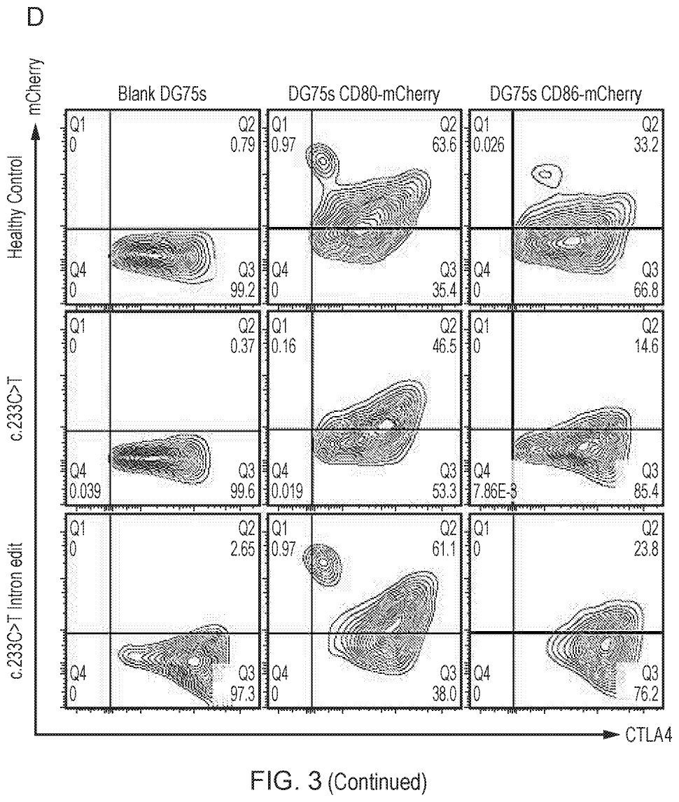

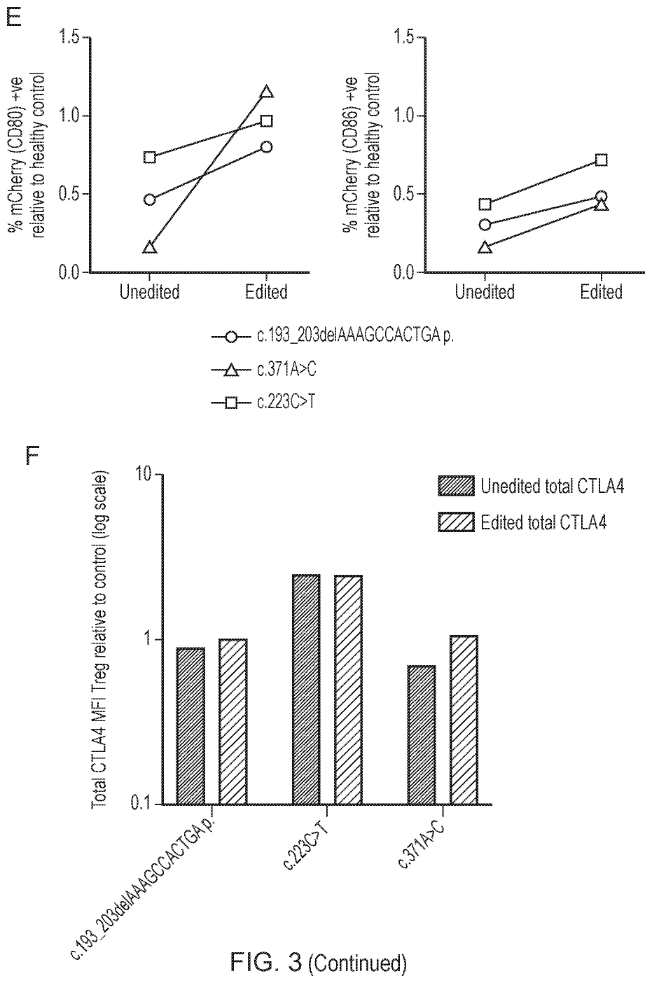

Similar or improved efficiencies of editing to the results in healthy donor T cells were obtained with HDR rates (determined by % GFP+) >60% in all patient samples tested (FIG. 3A). Editing restored CTLA4 surface expression to healthy donor levels (in mutants with reduced surface expression) (FIG. 3F). Patient cells have heterozygous mutations in CTLA4, and the differences in the rates of TE are best visualised in the T_{reg} fraction. Following overnight TE assay, cells were fixed and permeabilised and stained for total CTLA4 and the transcription factor FOXP3 so that TE could be examined in the T_{reg} fraction alone.

Cells from patients with three different mutations in CTLA4 were edited. In all three samples a reduction in TE compared to the healthy control was noted (FIGS. 3B, 3C, 3D, second rows). Following editing with the intronic strategy (WPRE-containing HDR donor) TE was restored to close to healthy donor levels (FIGS. 3B, 3C, 3D, bottom rows, and 3E).

### Example 10—Functional Assessment of Sorted Edited T_{regs} to Demonstrate Feasibility of Editing the T_{reg} Fraction Alone as a Therapeutic Approach

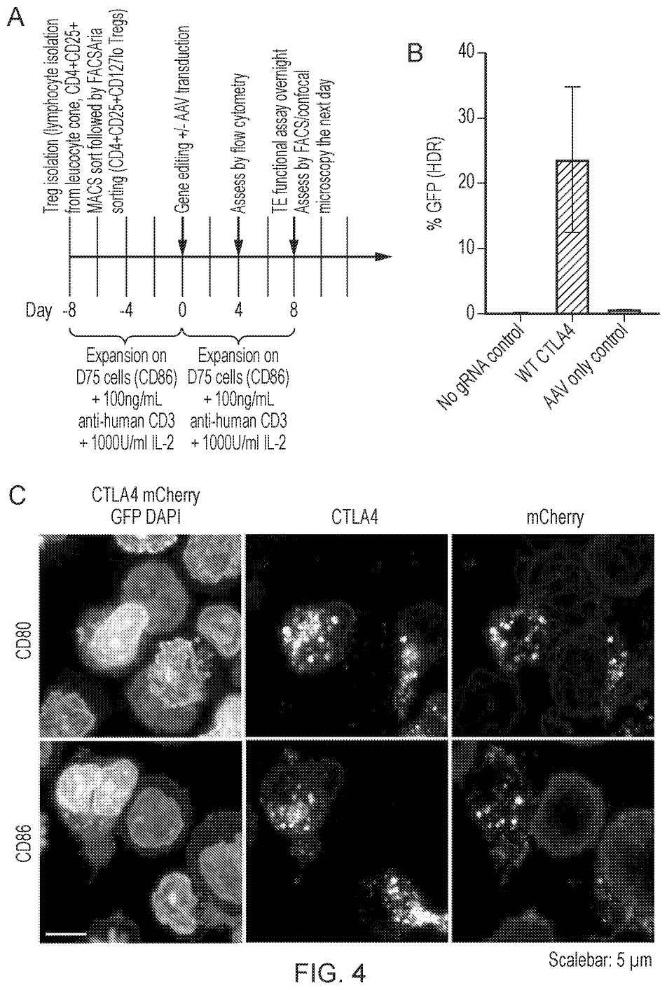

The profound autoimmunity seen in CTLA4 haploinsufficiency is the most problematic manifestation of the disorder. Given the emergence of T_{reg} therapies for other autoimmune conditions, editing the T_{reg} fraction alone was assessed, and the function of the edited T_{regs} examined. A T_{reg} based therapy for CTLA4 haploinsufficiency may be able to ‘rescue’ patients suffering with catastrophic autoimmune complications with minimal toxicity. Due to limitations on the number of patient samples available, experiments were performed on healthy donor T_{regs}. The experiment protocol was modified to facilitate expansion of T_{regs} with optimised culture conditions for this cell type (FIG. 4A). Using the intronic editing strategy (WPRE-containing HDR donor), HDR levels (determined by % GFP+) of up to 35% were achieved. Following editing, an overnight TE assay was performed. TE of CD80 and CD86 in the edited T_{regs} was equivalent to that observed in unedited T_{regs}. Further evidence of normal function of edited T_{regs} was obtained by performing confocal microscopy on the cells following overnight TE. Edited cells were identified by GFP positivity and colocalization of CTLA4, CD80 and CD86 (as observed in unedited cells) was demonstrated (FIG. 4C).

### Example 11—T Cell Gene Therapy for CTLA4 Insufficiency In Vivo Using a Murine Model

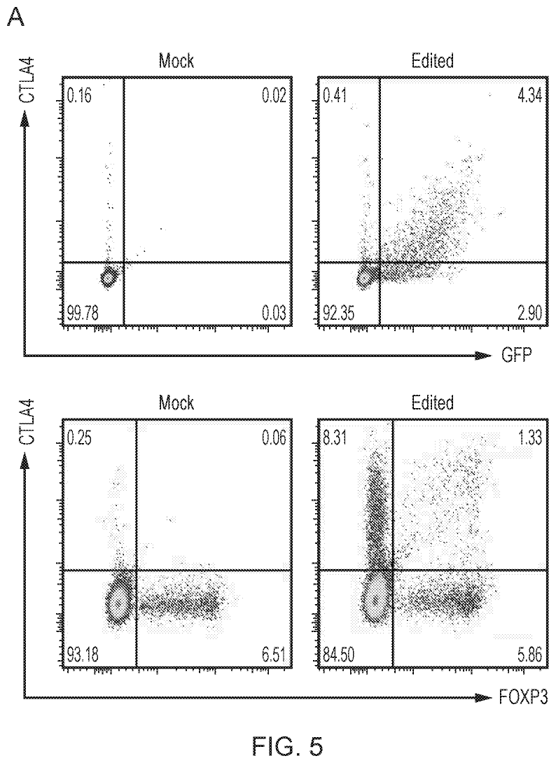

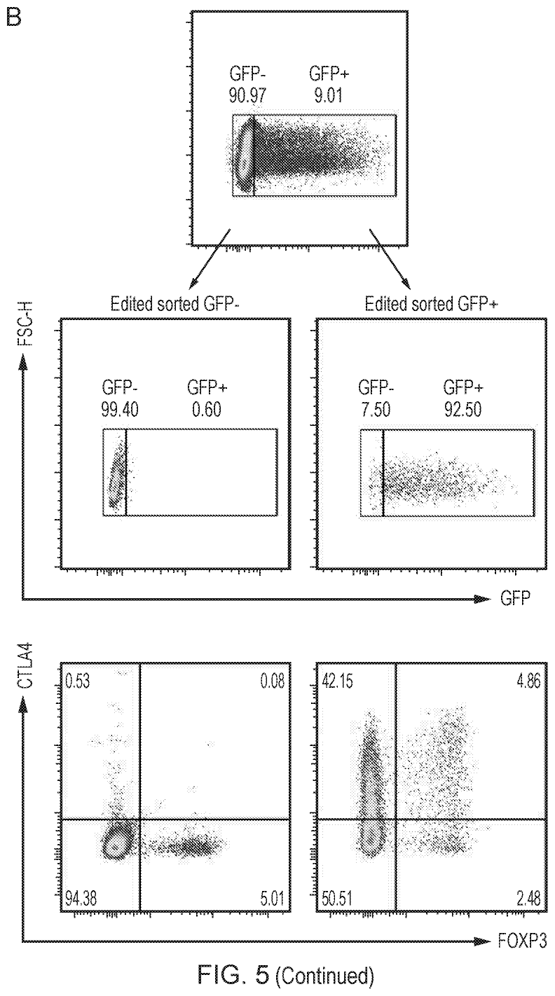

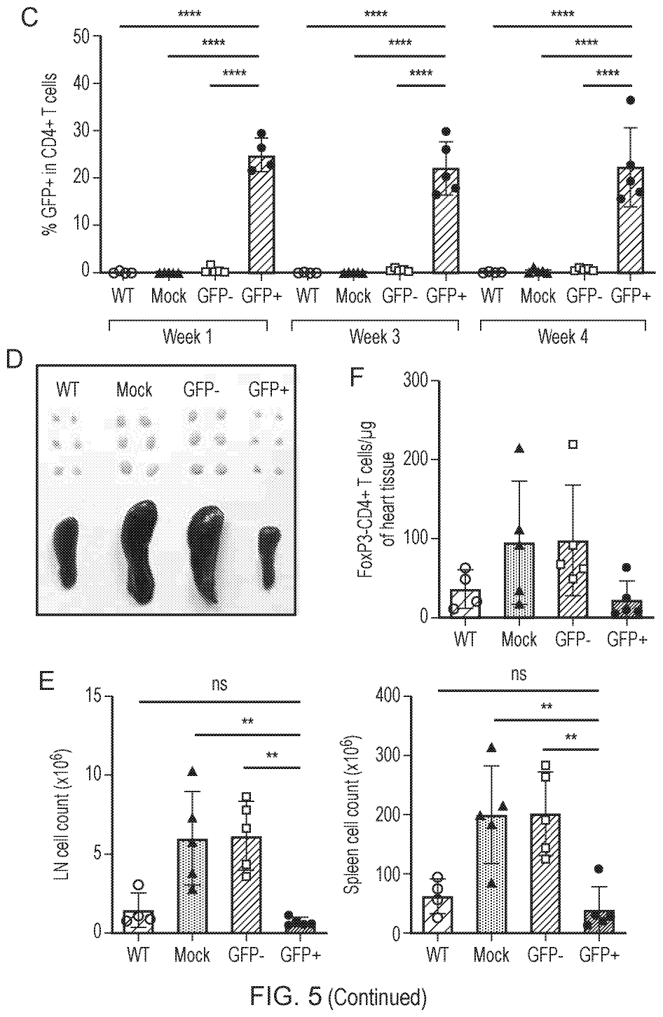

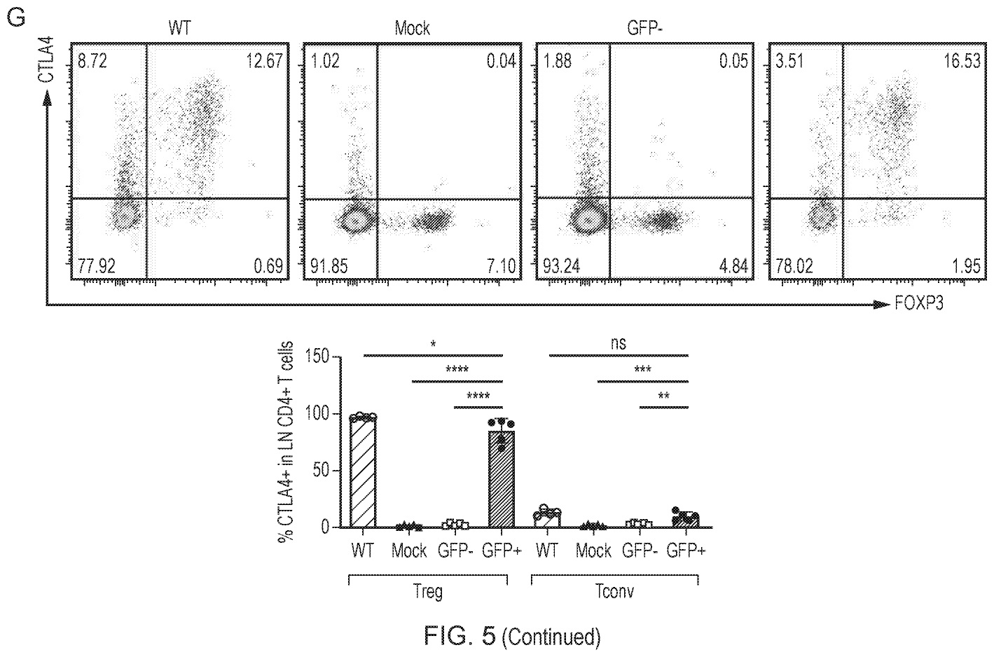

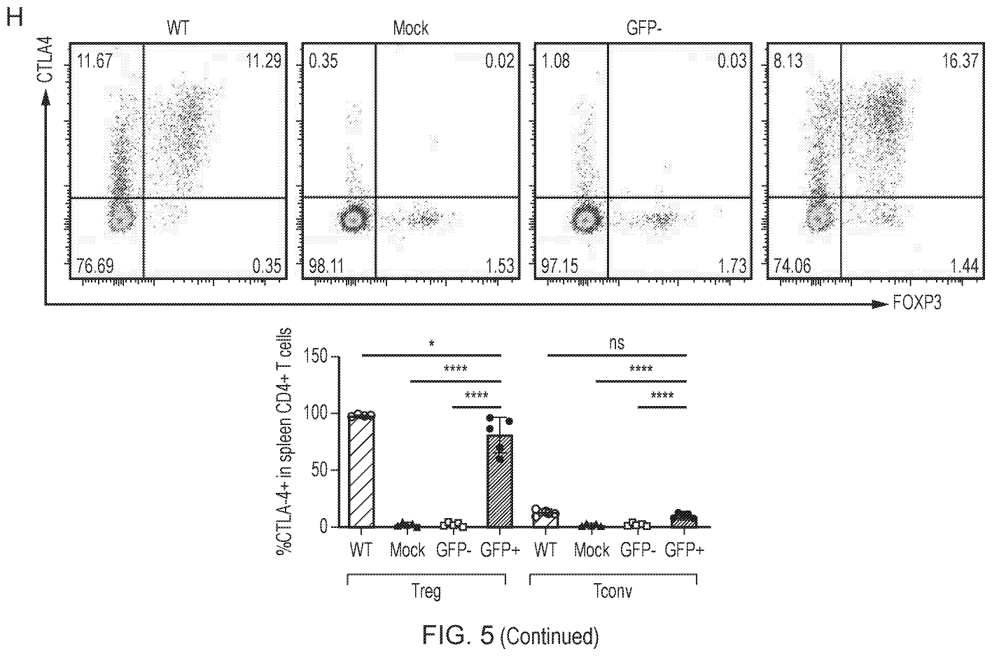

A gRNA was selected that causes a dsDNA break in the 3′ end of the first intron of murine CTLA4. An AAV6 HDR repair template was designed, replicating the architecture of the human template but containing the murine genomic sequence (donor 5). Editing efficiencies were lower than in human T cells however cycling CTLA4 molecules could only be detected in the GFP+ fraction of the CTLA4−/− cells, confirming successful gene expression (FIG. 5A, upper panel). Likewise, intracellular staining revealed restoration of CTLA4 expression in both Treg (Foxp3+) and Tconv (Foxp3−) compartments (FIG. 5A, lower panel). The lower editing efficiencies were mitigated by fluorescence-activated cell sorting (FACS) on GFP positivity to produce a cellular product with a high proportion of edited cells (FIG. 5B). The sorted GFP negative cells were used as a control population that were matched for activation/editing conditions having derived from the same wells as the GFP+ cells. Additional controls included mock-edited CTLA4−/− cells and unmanipulated WT T cells. 6×10⁵ edited or control cells were injected intravenously into Rag2−/− mice. Tail vein bleeds were performed 1, 3 and 4 weeks post adoptive transfer. In the mice which received the GFP+ edited cells a stable population of GFP+ cells were detectable at all timepoints demonstrating in vivo persistence as well as genetic stability (FIG. 5C).

All mice were sacrificed 4 weeks after cell transfer. To assess lymphoproliferation, the cellularity of peripheral lymph nodes and spleen were analyzed. When peripheral lymph nodes and spleens from all treatment groups were compared, lymphadenopathy and splenomegaly could be observed in mice that had received mock-edited and edited GFP-T cells (edited, but without repair) while lymph nodes and spleens from mice treated with edited GFP+ T cells did not differ from those found in the recipients of WT T cells (FIG. 5D). Furthermore, lymph nodes and spleens from recipients of edited GFP+ and WT T cells displayed equal cellularity whereas lymph nodes and spleens from mice treated with mock-edited and GFP− T cells contained significantly greater cell numbers (FIG. 5E). To assess lymphocytic organ infiltration, cardiac tissue was analysed: only mice from groups that had received mock-edited or GFP− T cells showed elevated cardiac tissue infiltration while Tconv numbers in mice treated with edited GFP+ cells did not exceed those seen in WT controls (FIG. 6F). Collectively, these findings indicated that the lymphoproliferative disease that occurred in recipients of CTLA4−/− cells was being controlled in mice that received edited GFP+ cells. Subsequent assessment of CTLA4 levels in cells from the lymph nodes (FIG. 5G) and spleens (FIG. 5H) of recipient mice revealed that CTLA4 was expressed in over 70% of lymph node Treg and over 60% of splenic Treg and in 6-15% of Tconv in mice that had received edited GFP+ T cells. Indeed, CTLA4 expression in edited GFP+ Treg was only marginally lower than that seen in WT Treg, while expression in edited Tconv was equivalent to WT levels.

Together these data demonstrated that CTLA4 edited T cells survived in vivo, expressed CTLA4 and were able to control the clinical phenotype of CTLA4 insufficiency, providing a powerful proof-of-principle.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of engineering a cell, comprising the steps of introducing into the cell:
i) a site-directed nuclease which is capable of cleaving a target nucleotide sequence at the 3′-end of Intron 1 of a Cytotoxic T-Lymphocyte Associated Protein 4 gene (CTLA4); and
ii) a nucleic acid construct which comprises a nucleic acid sequence comprising one or more of exon 2, exon 3 and exon 4 of CTLA4, or a sequence with at least 70% identity to exon 2, exon 3 and/or exon 4 of the CTLA4 gene, and 5′- and 3′-homology arms, wherein each of the 5′ and '3 homology arms is essentially complementary to a sequence flanking the target nucleotide sequence at the 3′-end of Intron 1 of CTLA4.

2. The method according to claim 1, wherein the target nucleotide sequence is within the sequence which corresponds to position 1268-2534, 1550-2400, 1900-2330 or 2097-2116 of SEQ ID No: 2.

3. The method according to claim 1 or claim 2 wherein the nuclease comprises a CRISPR-associated protein (Cas) and a gRNA molecule, wherein the gRNA is complementary to the target nucleotide sequence at the 3′-end of Intron 1 of CTLA4, preferably wherein the CRISPR-associated protein is Cas9.

4. The method according to claim 1 or claim 2, wherein the nuclease is a CRISPR-associated protein (Cas) in complex with a gRNA molecule, wherein the gRNA is complementary to the target nucleotide sequence at the 3′-end of Intron 1 of CTLA4, preferably wherein the CRISPR-associated protein is Cas9.

5. The method according to claim 3 or 4, wherein the gRNA comprises any one of SEQ ID Nos: 11, 14 or 17.

6. The method according to any of claims 1 to 5, wherein the nucleic acid construct is introduced into the cell in a vector, preferably wherein the vector is an adeno-associated virus (AAV) vector.

7. The method according to any of claims 1 to 6, wherein the cell is a haematopoietic stem cell (HSC) or CD3+ T cell, preferably a regulatory T cell.

8. The method according to any of claims 1 to 7, wherein the cell comprises a mutation in endogenous CTLA4 and/or is deficient in endogenous CTLA4 expression.

9. A site-directed nuclease which is capable of cleaving a target nucleotide sequence at the 3′-end of Intron 1 of CTLA4.

10. The site-directed nuclease according to claim 9, wherein the target nucleotide sequence is within the sequence which corresponds to position 1268-2534, 1550-2400, 1900-2330 or 2097-2116 of SEQ ID No: 2.

11. The site-directed nuclease according to claim 9 or claim 10, wherein the nuclease is a CRISPR-associated protein in complex with a gRNA molecule, wherein the gRNA is complementary to a nucleotide sequence at the 3′-end of Intron 1 of CTLA4, preferably wherein the CRISPR-associated protein is Cas9.

12. The site-directed nuclease according to claim 11, wherein the gRNA comprises any one of SEQ ID Nos: 11, 14 or 17.

13. A gRNA comprising any of SEQ ID Nos: 11, 14 or 17.

14. A vector comprising the gRNA of claim 13, preferably wherein the vector is an adeno-associated virus (AAV) vector.

15. A nucleic acid construct which comprises a nucleic acid sequence comprising one or more of exon 2, exon 3 and exon 4 of the CTLA4 gene, or a sequence with at least 70% identity to exon 2, exon 3 and/or exon 4 of the CTLA4 gene, and 5′- and 3′-homology arms, wherein each of the 5′ and 3′ the homology arms is essentially complementary to a sequence flanking a target nucleotide sequence at the 3′-end of Intron 1 of CTLA4.

16. The nucleic acid construct according to claim 15, wherein the target nucleotide sequence is within the sequence which corresponds to position 1268-2534, 1550-2400, 1900-2330 or 2097-2116 of SEQ ID No: 2.

17. A vector comprising the nucleic acid construct according to any of claims 15 to 16, preferably wherein the vector is an adeno-associated virus (AAV) vector.

18. A cell comprising the nucleic acid construct according to any of claims 15 to 16.

19. A cell engineered according to the method of any one of claims 1 to 8.

20. The cell according to claim 18 or 19, wherein the cell is a haematopoietic stem cell (HSC) or CD3+ T cell, preferably a regulatory T cell.

21. A pharmaceutical composition comprising the cell according to any of claims 18 to 20.

22. A method of treating and/or preventing a disease in a subject comprising administering the cell according to any of claims 18 to 20.

23. Use of a cell according to any of claims 18 to 20 in the manufacture of a medicament for the treatment and/or prevention of a disease.

24. A cell according to any of claims 18 to 20, for use in treating and/or preventing disease in a subject.

25. The method according to claim 22, use according to claim 23, or cell for use according to claim 24, wherein the disease is an immune deficiency disease, autoimmune disease, cancer or disease associated with solid organ and/or haematopoietic stem cell transplantation.

26. The method, use, or cell for use according to claim 25, wherein the disease is common variable immune deficiency (CVID), hypogammaglobulinemia, recurrent infections, granulomatous lymphocytic interstital lung disease (GLILD), fibrosis, bronchiectasis, type 1 diabetes, autoimmune thyroiditis, arthritis, rheumatoid arthritis, juvenile idiopathic arthritis, psoriasis, psoriatic arthritis, lupus, uveitis, vitiligo, myasthenia gravis, immune thrombocytopenia, enteropathy, autoimmune hemolytic anemia, autoimmune neutropenia, acute lymphoblastic leukemia (ALL), acute myelogenous leukemia (AML), chronic lymphocytic leukemia (CLL), chronic myelogenous leukemia (CML), acute monocytic leukemia (AMOL), Hodgkin's lymphoma, non-Hodgkin's lymphoma, transplant rejection and/or graft-versus-host disease (GvHD).


SEQUENCE LISTING (31 sequences)

SEQ ID NO: 1 - DNA, 6195 bp, Homo sapiens
  source 1..6195: mol_type = genomic DNA
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaaggtgagtgagacttttggagcatgaagatggaggaggtgtttctcctacctgggtttcatttgtttcagcagtcaaaggcagtgatttatagcaaagccagaagttaaaggtaaaactccaatctggcttggctggctctgtattccagggccagcagggagcagttgggcggcagcaaataaggcaaagagatagctcagaacagagcgccaggtatttagtaggggcttcatgaatgcatgtgagttggtttagtagagagacacaggcaatttcagacccttctatgagactggaagtgatttaagagggaaaggatagccatagtcctgaatacatttgagctgggtttcaggatgagctcacaagttcctttaaaaaaaattgacttaagcaaatcctgggaagagtttttttgctatacaattcaaggttttaaggtcctcggattcatatactttataaatgaattagccagcttgtttaaaatgtagggaaattgtgggaagaatgccttctttacttaattcaaggttttaaggttctcttaatcaattctactagctaattagccaattatttaaaaataaaagtttgaaattgccaaaaaaaaaagacaaggaaaaggaaagaaagaaagccaccagtctgtttggcatacaatacttaattgttgcctgacctacgtgtgggtttcagatgcagatcctcagttttcagctcttcagagactgacaccaggtttgttacacggcttaaaatgatgagtatatccattgaatctcaaccttatctctctctagaccttcttggttaagaaaccatgtagtttgtatgaagtaggtactcaaaagatatttgatgatttaatttttactggagaagaaatattcatatatgttttcttatttttacatgttttaaatatgtaaagattaaataaacactcttagaagtatttaaatttcctaaagtaaatttatctcaaccagtaacaggaccctcccaatactggaaagttgagtgtgaccgcatttagtggtgatgagtgtgagcttgcttggggagagggcaggacatttaggatttcttaagcttagagtcaatacaataaagattattgagtgctcacttgggtgggctataatcactgctcacaggagttcatgaaccacaagtaaaagagtgaggagatatgattagctcacaaataactttaatacagagcagaaagtaatgaactactgcaatggagttatcacagtgctaaggatgctcagagggcatctctgataggcagaggtgagggttagggaaggaagctgtagtctagctagctagagctgctggaatagacatgacaatggctgctgccaaactgttttctcttctgaggacagatgtcccgtgcaagtggcttggtggaagggactagtgtctctaatatagggtgatttataagcaggaaagtgtgtcctagaaattcagaccagagtgatagattggaattggatcatgggggactcattgaatgttatttattgtatttgtttttgcgatcagtgttagtaaagtgtcaaagggattgagcagatgagtgacatcatgcaacacaagttttgagtttcacttgtcagactgactggagaggggcctggttagttacaggaaggtaatttggcatgcagccactatttttgagttgatgcaagcctctctgtatggagagctggtctcctttatcctgtgggaaaagagaacaaaggagcatgggagtgttcaagggaaggagaaataaagggcagagaggcagcggtggtgtcaggggaagcccacaggagttaacagcagggttgcctcaacctagagaggaagcgacctggtgccctcggctctgtggcttccttcatctaacaacatcttccactctacaacaatgccagggaaggcggaggctggtacagtgcatcaagacacagctactcctgggtgacagaggttcagggccagctcactaagtaggcagaagtttttgacatatactttgagagataaagcaagattctgtacctcaaccttcagaatttcccctaccactcattatagttccggagctatatagctcctatcattctatcataaccttagaataccagagaacatatcatctcatctaattatctcttactatatgtgaaaaaaatgaaggacatgggggaagtgtgacttgccccaaatcacatatttcatggtagagccaggtcttctgtttgtcatatcagtgttcttcctgccacaaccatcttgaagaatctatttctcagtaagaaaatatctttatggagagtagctggaaaacagttgagagatggaggggaggctgggggtgtggagaggggaaggggtaagtgatagattcgttgaaggggggagaaaaggccgtggggatgaagctagaaggcagaagggcttgcctgggcttggccatgaaggagcatgagttcactgagttccctttggcttttccatgctagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattggtgagcaaagccatttcactgagttgacacctgttgcattgcagtcttctatgcacaaaaacagttttgttccttaatttcaggaggtttacttttaggactgtggacattctctttaagagttctgtaccacatggtagccttgcttattgtgggtggcaaccttaatagcattctgactgtaaaataaaatgatttggggaagttggggctctcgctctggagtgctaaccatcatgacgtttgatctgtacttttgatatgatatgatgctcctggggaagtagtcccaaatagccaaacctattggtgggctacccatgcaatttaggggtggacctcaaggcctggaagctctaatgtccttttttcaccaatgttggggagtagagccctagagtttaaaactgtctcagggaggctctgctttgttttctgttgcagatccagaaccgtgcccagattctgacttcctcctctggatccttgcagcagttagttcggggttgtttttttatagctttctcctcacagctgtttctttgagcaaaatggtgagtgtggtgctgatggtgcaccatgtctgatggggatacctttagtggtatcaactggccaaaagatgatgttgagtttagtgttcttgagatgagatgaggcaataaatgaagaggaaggacagtggtaaagaacgcactagaaccgtaggcattggcatttgaggtttcagaatgactaatattttagatgaatttgtttgacattgaatgttcatgtgcttctgagcagggtttcaatttgagtaaccgttgcaataacatggggcagctgttttgctctttgtcttcatgacaactgtacttaagctaacagccctgaaacatgagattaggctgggcagaatgctgctagagaggaccacttggatggtctttattctccttctccatgtccctctccatcacctggaagtcacctctgggtgccactctggtgccttccttgtcgaagctgtagctgctcacatgacacctatccctgttatccagtttgcttgactgggacgttttgccttccccttcagccaggaagtgaaagtcccagtttttatttatcacaggtgttggtattggtggtagaagaggtagaattatggaatcaggcctcctgtcaggatttctttttgacagtccctctcagacacctctgcctaaggccagctttgccattacaaactctcccttctccctctctcccttcttctcttcctcttccttcttctcgctctttctctctctctctttctccctctctgtctcttatacacatacacaaagatatactctattccaacatcctctacccaacctgacagagatgtcctttgctgtaggttcagcagtggggatgagaaatacagctctcaaacaggataactaaagcttattatcttatcaagcttgttcccttgcagacaagattgatcaattatcataggctttctgggtgttctttctgaagctttctcaaagtctctttctcctatcttccattcaaggcaaatgattgccatttaacatcaaaatcacagttatttatctaaaataaattttaatagctgaatcaagaaaatctcctgaggtttataattctgtatgctgtgaacattcatttttaaccagctagggacccaatatgtgttgagttctattatggttagaagtggcttccgtattcctcagtagtaattactgtttctttttgtgtttgacagctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 2 - DNA, 2534 bp, Homo sapiens
  source 1..2534: mol_type = genomic DNA
gtgagtgagacttttggagcatgaagatggaggaggtgtttctcctacctgggtttcatttgtttcagcagtcaaaggcagtgatttatagcaaagccagaagttaaaggtaaaactccaatctggcttggctggctctgtattccagggccagcagggagcagttgggcggcagcaaataaggcaaagagatagctcagaacagagcgccaggtatttagtaggggcttcatgaatgcatgtgagttggtttagtagagagacacaggcaatttcagacccttctatgagactggaagtgatttaagagggaaaggatagccatagtcctgaatacatttgagctgggtttcaggatgagctcacaagttcctttaaaaaaaattgacttaagcaaatcctgggaagagtttttttgctatacaattcaaggttttaaggtcctcggattcatatactttataaatgaattagccagcttgtttaaaatgtagggaaattgtgggaagaatgccttctttacttaattcaaggttttaaggttctcttaatcaattctactagctaattagccaattatttaaaaataaaagtttgaaattgccaaaaaaaaaagacaaggaaaaggaaagaaagaaagccaccagtctgtttggcatacaatacttaattgttgcctgacctacgtgtgggtttcagatgcagatcctcagttttcagctcttcagagactgacaccaggtttgttacacggcttaaaatgatgagtatatccattgaatctcaaccttatctctctctagaccttcttggttaagaaaccatgtagtttgtatgaagtaggtactcaaaagatatttgatgatttaatttttactggagaagaaatattcatatatgttttcttatttttacatgttttaaatatgtaaagattaaataaacactcttagaagtatttaaatttcctaaagtaaatttatctcaaccagtaacaggaccctcccaatactggaaagttgagtgtgaccgcatttagtggtgatgagtgtgagcttgcttggggagagggcaggacatttaggatttcttaagcttagagtcaatacaataaagattattgagtgctcacttgggtgggctataatcactgctcacaggagttcatgaaccacaagtaaaagagtgaggagatatgattagctcacaaataactttaatacagagcagaaagtaatgaactactgcaatggagttatcacagtgctaaggatgctcagagggcatctctgataggcagaggtgagggttagggaaggaagctgtagtctagctagctagagctgctggaatagacatgacaatggctgctgccaaactgttttctcttctgaggacagatgtcccgtgcaagtggcttggtggaagggactagtgtctctaatatagggtgatttataagcaggaaagtgtgtcctagaaattcagaccagagtgatagattggaattggatcatgggggactcattgaatgttatttattgtatttgtttttgcgatcagtgttagtaaagtgtcaaagggattgagcagatgagtgacatcatgcaacacaagttttgagtttcacttgtcagactgactggagaggggcctggttagttacaggaaggtaatttggcatgcagccactatttttgagttgatgcaagcctctctgtatggagagctggtctcctttatcctgtgggaaaagagaacaaaggagcatgggagtgttcaagggaaggagaaataaagggcagagaggcagcggtggtgtcaggggaagcccacaggagttaacagcagggttgcctcaacctagagaggaagcgacctggtgccctcggctctgtggcttccttcatctaacaacatcttccactctacaacaatgccagggaaggcggaggctggtacagtgcatcaagacacagctactcctgggtgacagaggttcagggccagctcactaagtaggcagaagtttttgacatatactttgagagataaagcaagattctgtacctcaaccttcagaatttcccctaccactcattatagttccggagctatatagctcctatcattctatcataaccttagaataccagagaacatatcatctcatctaattatctcttactatatgtgaaaaaaatgaaggacatgggggaagtgtgacttgccccaaatcacatatttcatggtagagccaggtcttctgtttgtcatatcagtgttcttcctgccacaaccatcttgaagaatctatttctcagtaagaaaatatctttatggagagtagctggaaaacagttgagagatggaggggaggctgggggtgtggagaggggaaggggtaagtgatagattcgttgaaggggggagaaaaggccgtggggatgaagctagaaggcagaagggcttgcctgggcttggccatgaaggagcatgagttcactgagttccctttggcttttccatgctag

SEQ ID NO: 3 - DNA, 348 bp, Homo sapiens
  source 1..348: mol_type = genomic DNA
caatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattg

SEQ ID NO: 4 - DNA, 110 bp, Homo sapiens
  source 1..110: mol_type = genomic DNA
atccagaaccgtgcccagattctgacttcctcctctggatccttgcagcagttagttcggggttgtttttttatagctttctcctcacagctgtttctttgagcaaaatg

SEQ ID NO: 5 - DNA, 1258 bp, Homo sapiens
  source 1..1258: mol_type = genomic DNA
ctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 6 - DNA, 6808 bp, Mus musculus
  source 1..6808: mol_type = genomic DNA
ctacacatatgtagcacgtaccttggatcaaagctgtctatataaagtccccgagtctgtgtgggttcaaacacatctcaaggcttctggatcctgttgggttttactctgctccctgaggacctcagcacatttgccccccagccatggcttgtcttggactccggaggtacaaagctcaactgcagctgccttctaggacttggccttttgtagccctgctcactcttcttttcatcccagtcttctctgaaggtgagtggagacttctggaacatggaggtggaggaggtgtttttcctacatgggtttcgttttttttttttctttcagaagctgaggacagtgatttatagaaatgccagaagttaaagccaaaccctctactttttggtttaacagcttcctacagacaagttccaagaagaatataggaggtctagcagtagcgaaggagctgagactagccacagcagcttgctgggatctcagctgggacttaaggaaccatgagagttccttaagtcccatatgggggagaacaagttctttgtcactgctagaaaatcctgtaaagttaagagatttaaggggggcatttgaaatcgtgagtacatttgatcttgatatcacagtaacattaaggtttttctccttacccccaatcccacccccaaaacttacctaaattcaaataaataagaagttttcctcattcactttaagattttgagacactcttagttctttaaattatcacacctaaggcttaggggacatagcagaagagggggcagaaagactatgagagccaaaggtacatagagtttactgagagattgtgtctcttaggatgtcaaatggtacacccataaagtctcaccaacacgactgcctaaacttgagctgaataaggacatgaataacaatagacatacccaagtggatggggaaagaccaagatgcttcagcactatagcaataactacaggaagccaaggaatatggagagtaggaaattgccaagggaaaagcacaccagctgactatccaataacatatggtcagccttcaaaacaaaaagcaaataacatatagaatgggcaggtttcattaagaatgactataactatagtctgtgcatatcacaacaaataatgaaaaggggggccatgaatttgaaagaaaagcaaggaggggtacatgagaaggtttggaggagagaaaggagaaataatgtaattatattaatctcaaagaatagaagaagtagtaagtaaagggaaaataaattagttaattattttaaagatggaatagttttaagtcgaaggaagaaagaaaggcaaagtgatagaaagacaagaaaaggggaggaaggaagagagaagagaggaaggaagaaaggaaggaaggaaggaagggaggaagggagggaaaaagaaagaagagagaagggaggaggacaggaaggagggagggagggagggaggaagggagaaagggagaaaggaagaaaacaattatcagctttttggcctaagagtctttttattattttttttcttcataaaatccttaattgtttcattaactatccatggatttttaagtacgaatccctgttttttcagatgaaatagaaacttttgaaaggctaataccaggcttgttatgtgccttgagatgtacatatctactggaccctgagcatctctctctagatcttttaaattaagaaatcacatagtttgtgtgacactgatgcacaaatgatatttgataatctaattttactaaataagtcatggacagtttcttgtctgaataggccacaaaaactttaatgtgggagagaacattttttaaacactctagtggggtttccatgagcaaagggtgcttgcagaaagtctctgatagtagagatgaaggctaggcagacacctgctgtttcacccgctaagctgatggagtaaccatggcaactgccaccatattgttctcttttctgaggacagatgctaatcagtacaggtgctttcagaagagactagggtatctatatagcctggtttatggataggagaggtggtcttggaaactaagcctggggtagtattcaagattgcaatacactgaaaactaattattgtcttgtttttacaatctatgttagtaaactaccaatgacattgttcagtttaagttttgggtgtaatcttcaatactgaccggaaaacatccaggttagttatgaaaaggcaatatgacagaaagccacttttgtgtgctgagagtacaacccgagatcgtgtgtattctaggcaagcactctaccaccgacctacatctccagcccttctgcctgtggttctttgtcttgtaaagcaatgtcttgttgtttagctgatgctggccttgcacttgctatgtagccttcatctgctggctgctaggactgcagatttgtaccaccaataccggactgcaaacccactaatttctaatgatgaaggcatgtttgtatagagagctggactcctttcttctgtagtatacagggaggaaagagaaaacaacaaaaacagcagcagcagcagcagcaacaacaacaacaacaaaaaccccaaggacaaggaaagtgttaagtgaaggaaagaagggaggcagaagaggtggcagggaagcaggggaagcccacagaagttaaagcagggttgtctcaacccagagaggaaatgaccctggtgccctcagctctgtggcttccttgactgatgtatacaccactctaccacagtgatgccaggaaaagggtgaccaatgcattgacctgaggttcaactgctcctggttgacagaggtacgcttataaataagtaggtaggaaaattttgaagcttactttgagagatgaggcaaggttctgcacctcaagctccaggaatgtctcgactgccattcactatgtttcctgcgtgatatagttctattatcaccaaagaaggcgctgtactgacatgtaggctacccccttttcttactgcaggggagaataaatgaaaggaagaattatttgccaaaatgacacattttatgagagccagatcttctttttgctataccagtattctccttgccatagccaactgtcttcaataaactatcaataaggggatcttggagagtgactgactacagctgaaagatgggaagtggagtgccagggtggatgggtggagaggcaaagggtgaagggagtgatgagtttgttgaggggtgagcttgcaggagttcatccaagatgaacctcccctggcctcaggtgtggcctaatagttcaaaccgtggatgatcatgagcccactaagtgccctttggactttccatgtcagccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattggtgagcaaagccattccactaagaacaagtctgttgcattattattgtctttacaccagaatagttttgttccttggtttggagtccttcatagttaggtctgtgatgcatagctaggaattccctagtagatagtagtcttgcttatactgagaagttacataaccatcactctgattgcaatgaaacgcatttggggatgtgttttttatactgcttgctgatagtctaggacacttgttcttgaagtttagtcttgtccctttgatggcactctgggaaagtcatgtattaaataagtagccagacttccctatagtttaccaatacaagttagggttgactagcaaaacctggaacctctaacttccttttactacccatgaggaactaggaccccacaattggaaactctctcaggaggttgatgcttcgtcttctgttgcagatccagaaccatgcccggattctgacttcctcctttggatccttgtcgcagttagcttggggttgtttttttacagtttcctggtcactgctgtttctttgagcaagatggtgagtgtgatgttgacgtttccccacagttaatggggatacttttagttgtaccctactgaccaattggtgttgagttgaagcaataaacaaggagcaggaaggatagggtaaagaacacgctagaaccccatgcacttgccttagaggtttcgggatgactaatactgtacgtgagcatgtttgacagtgaatgtttgtgtgcttctgagcagggtttcagtttgagtaactgtttgaacaacatggagcagctgttttggttgtcactgtcatggcaatgtccttaatcctaggacacacagcagtctctgggcaaccctttctagttagaaccacctagatggatttttgtcctttaccaagcaccatctcttggtccctcttgtgtcttctttgttcaaggcatagatacttgtagaatacccatccccattatctatgtacttacttaccttgccttgatgccctccctagctcagtgaatgaaagcccttgtttttatttagtatattggcagtgaaaagagagaagtgtagaatcaagtctccagtcagggttctgaacagtcctccaccatgtctgtgtaaggctggctttccttatcacaagcaatatcctttgaccccatctcttctcttctttttcttactcttcctactcctccattttcctcatctctctacctttgccactttctgtatctctttctgcatctatattcacacacacacacacacacacacacacacacacacacacacacacacatacacacacactttcacagaatctttgttctgttgctgtcttccttttttatctaccattaagctaccacccaggctggctgactagacatccattgttgaagtgttgaggtgacagagactggaaactaacacagacaaagaaaaagcactaacaaacataagaggaatggagatgcagaattcagcttcaaagcaggttatcagagccaccttcaaatatttcaggtgctttgattaccacagacacgatctagccaactatcttgggcattttggttgttcttggtgaagttgtctcaaagcccctttctcccttttgcagaattatttatctaaggtgaatttgactagctcagttaagaaaatctcccagagtttataattctgtatgtcgttgtggatgttttatttttgctggggagggacccgatatttactgggtcctattatgattgcaattggcttctatattctttgataataattgccttttaaaaaatgttttttgcagctaaagaaaagaagtcctcttacaacaggggtctatgtgaaaatgcccccaacagagccagaatgtgaaaagcaatttcagccttattttattcccatcaactgaaaggccgtttatgaagaagaaggagcatacttcagtctctaaaagctgaggcaatttcaactttccttttctctccagctatttttacctgtttgtatattttaaggagagtatgcctctctttaatagaaagctggatgcaaaattccaattaagcatactacaatttaaagctaaggagcatgaacagagagctgggatatttctgttgtgtcagaaccattttactaaaagcatcacttggaagcagcataaggatatagcattatggtgtggggtcaagggaacattagggaatggcacagcccaaagaaaggaagggggtgaaggaagagattatattgtacacatcttgtatttacctgagagatgtttatgacttaaataatttttaaatttttcatgctgttattttctttaacaatgtataattacacgaaggtttaaacatttattcacagagctatgtgacatagccagtggttccaaaggttgtagtgttccaagatgtatttttaagtaatattgtacatgggtgtttcatgtgctgttgtgtatttgctggtggtttgaatataaacactatgtatcagtgtcgtcccacagtgggtcctggggaggtttggctggggagcttaggacactaatccatcaggttggactcgaggtcctgcaccaactggcttggaaactagatgaggctgtcacagggctcagttgcataaaccgatggtgatggagtgtaaactgggtctttacactcattttattttttgtttctgcttttgttttcttcaatgatttgcaaggaaaccaaaagctggcagtgtttgtatgaacctgacagaacactgtcttcaaggaaatgcctcattcctgagaccagtaggtttgtttttttaggaagttccaatactaggaccccctacaagtactatggctcctcgaaaacacaaagttaatgccacaggaagcagcagatggtaggatgggatgcacaagagttcctgaaaactaacactgttagtgttttttttttaactcaatattttccatgaaaatgcaaccacatgtataatatttttaattaaataaaagtttcttgtgattgtttt

SEQ ID NO: 7 - DNA, 3143 bp, Mus musculus
  source 1..3143: mol_type = genomic DNA
gtgagtggagacttctggaacatggaggtggaggaggtgtttttcctacatgggtttcgttttttttttttctttcagaagctgaggacagtgatttatagaaatgccagaagttaaagccaaaccctctactttttggtttaacagcttcctacagacaagttccaagaagaatataggaggtctagcagtagcgaaggagctgagactagccacagcagcttgctgggatctcagctgggacttaaggaaccatgagagttccttaagtcccatatgggggagaacaagttctttgtcactgctagaaaatcctgtaaagttaagagatttaaggggggcatttgaaatcgtgagtacatttgatcttgatatcacagtaacattaaggtttttctccttacccccaatcccacccccaaaacttacctaaattcaaataaataagaagttttcctcattcactttaagattttgagacactcttagttctttaaattatcacacctaaggcttaggggacatagcagaagagggggcagaaagactatgagagccaaaggtacatagagtttactgagagattgtgtctcttaggatgtcaaatggtacacccataaagtctcaccaacacgactgcctaaacttgagctgaataaggacatgaataacaatagacatacccaagtggatggggaaagaccaagatgcttcagcactatagcaataactacaggaagccaaggaatatggagagtaggaaattgccaagggaaaagcacaccagctgactatccaataacatatggtcagccttcaaaacaaaaagcaaataacatatagaatgggcaggtttcattaagaatgactataactatagtctgtgcatatcacaacaaataatgaaaaggggggccatgaatttgaaagaaaagcaaggaggggtacatgagaaggtttggaggagagaaaggagaaataatgtaattatattaatctcaaagaatagaagaagtagtaagtaaagggaaaataaattagttaattattttaaagatggaatagttttaagtcgaaggaagaaagaaaggcaaagtgatagaaagacaagaaaaggggaggaaggaagagagaagagaggaaggaagaaaggaaggaaggaaggaagggaggaagggagggaaaaagaaagaagagagaagggaggaggacaggaaggagggagggagggagggaggaagggagaaagggagaaaggaagaaaacaattatcagctttttggcctaagagtctttttattattttttttcttcataaaatccttaattgtttcattaactatccatggatttttaagtacgaatccctgttttttcagatgaaatagaaacttttgaaaggctaataccaggcttgttatgtgccttgagatgtacatatctactggaccctgagcatctctctctagatcttttaaattaagaaatcacatagtttgtgtgacactgatgcacaaatgatatttgataatctaattttactaaataagtcatggacagtttcttgtctgaataggccacaaaaactttaatgtgggagagaacattttttaaacactctagtggggtttccatgagcaaagggtgcttgcagaaagtctctgatagtagagatgaaggctaggcagacacctgctgtttcacccgctaagctgatggagtaaccatggcaactgccaccatattgttctcttttctgaggacagatgctaatcagtacaggtgctttcagaagagactagggtatctatatagcctggtttatggataggagaggtggtcttggaaactaagcctggggtagtattcaagattgcaatacactgaaaactaattattgtcttgtttttacaatctatgttagtaaactaccaatgacattgttcagtttaagttttgggtgtaatcttcaatactgaccggaaaacatccaggttagttatgaaaaggcaatatgacagaaagccacttttgtgtgctgagagtacaacccgagatcgtgtgtattctaggcaagcactctaccaccgacctacatctccagcccttctgcctgtggttctttgtcttgtaaagcaatgtcttgttgtttagctgatgctggccttgcacttgctatgtagccttcatctgctggctgctaggactgcagatttgtaccaccaataccggactgcaaacccactaatttctaatgatgaaggcatgtttgtatagagagctggactcctttcttctgtagtatacagggaggaaagagaaaacaacaaaaacagcagcagcagcagcagcaacaacaacaacaacaaaaaccccaaggacaaggaaagtgttaagtgaaggaaagaagggaggcagaagaggtggcagggaagcaggggaagcccacagaagttaaagcagggttgtctcaacccagagaggaaatgaccctggtgccctcagctctgtggcttccttgactgatgtatacaccactctaccacagtgatgccaggaaaagggtgaccaatgcattgacctgaggttcaactgctcctggttgacagaggtacgcttataaataagtaggtaggaaaattttgaagcttactttgagagatgaggcaaggttctgcacctcaagctccaggaatgtctcgactgccattcactatgtttcctgcgtgatatagttctattatcaccaaagaaggcgctgtactgacatgtaggctacccccttttcttactgcaggggagaataaatgaaaggaagaattatttgccaaaatgacacattttatgagagccagatcttctttttgctataccagtattctccttgccatagccaactgtcttcaataaactatcaataaggggatcttggagagtgactgactacagctgaaagatgggaagtggagtgccagggtggatgggtggagaggcaaagggtgaagggagtgatgagtttgttgaggggtgagcttgcaggagttcatccaagatgaacctcccctggcctcaggtgtggcctaatagttcaaaccgtggatgatcatgagcccactaagtgccctttggactttccatgtcag

SEQ ID NO: 8 - DNA, 348 bp, Mus musculus
  source 1..348: mol_type = genomic DNA
ccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattg

SEQ ID NO: 9 - DNA, 110 bp, Mus musculus
  source 1..110: mol_type = genomic DNA
atccagaaccatgcccggattctgacttcctcctttggatccttgtcgcagttagcttggggttgtttttttacagtttcctggtcactgctgtttctttgagcaagatg

SEQ ID NO: 10 - DNA, 1220 bp, Mus musculus
  source 1..1220: mol_type = genomic DNA
ctaaagaaaagaagtcctcttacaacaggggtctatgtgaaaatgcccccaacagagccagaatgtgaaaagcaatttcagccttattttattcccatcaactgaaaggccgtttatgaagaagaaggagcatacttcagtctctaaaagctgaggcaatttcaactttccttttctctccagctatttttacctgtttgtatattttaaggagagtatgcctctctttaatagaaagctggatgcaaaattccaattaagcatactacaatttaaagctaaggagcatgaacagagagctgggatatttctgttgtgtcagaaccattttactaaaagcatcacttggaagcagcataaggatatagcattatggtgtggggtcaagggaacattagggaatggcacagcccaaagaaaggaagggggtgaaggaagagattatattgtacacatcttgtatttacctgagagatgtttatgacttaaataatttttaaatttttcatgctgttattttctttaacaatgtataattacacgaaggtttaaacatttattcacagagctatgtgacatagccagtggttccaaaggttgtagtgttccaagatgtatttttaagtaatattgtacatgggtgtttcatgtgctgttgtgtatttgctggtggtttgaatataaacactatgtatcagtgtcgtcccacagtgggtcctggggaggtttggctggggagcttaggacactaatccatcaggttggactcgaggtcctgcaccaactggcttggaaactagatgaggctgtcacagggctcagttgcataaaccgatggtgatggagtgtaaactgggtctttacactcattttattttttgtttctgcttttgttttcttcaatgatttgcaaggaaaccaaaagctggcagtgtttgtatgaacctgacagaacactgtcttcaaggaaatgcctcattcctgagaccagtaggtttgtttttttaggaagttccaatactaggaccccctacaagtactatggctcctcgaaaacacaaagttaatgccacaggaagcagcagatggtaggatgggatgcacaagagttcctgaaaactaacactgttagtgttttttttttaactcaatattttccatgaaaatgcaaccacatgtataatatttttaattaaataaaagtttcttgtgattgtttt

SEQ ID NO: 11 - RNA, 20 nt, synthetic construct
  source 1..20: mol_type = other RNA
agctccggaactataatgag

SEQ ID NO: 12 - DNA, 20 bp, synthetic construct
  source 1..20: mol_type = other DNA
agctccggaactataatgag

SEQ ID NO: 13 - DNA, 20 bp, synthetic construct
  source 1..20: mol_type = other DNA
ctcattatagttccggagct

SEQ ID NO: 14 - RNA, 20 nt, synthetic construct
  source 1..20: mol_type = other RNA
gagaaatagattcttcaaga

SEQ ID NO: 15 - DNA, 20 bp, synthetic construct
  source 1..20: mol_type = other DNA
gagaaatagattcttcaaga

SEQ ID NO: 16 - DNA, 20 bp, synthetic construct
  source 1..20: mol_type = other DNA
tcttgaagaatctatttctc

SEQ ID NO: 17 - RNA, 20 nt, synthetic construct
  source 1..20: mol_type = other RNA
gatatgacaaacagaagacc

SEQ ID NO: 18 - DNA, 20 bp, synthetic construct
  source 1..20: mol_type = other DNA
gatatgacaaacagaagacc

SEQ ID NO: 19 - DNA, 20 bp, synthetic construct
  source 1..20: mol_type = other DNA
ggtcttctgtttgtcatatc

SEQ ID NO: 20 - DNA, 2939 bp, synthetic construct
  source 1..2939: mol_type = other DNA
gcttttaattaaatttttgagttgatgcaagcctctctgtatggagagctggtctcctttatcctgtgggaaaagagaacaaaggagcatgggagtgttcaagggaaggagaaataaagggcagagaggcagcggtggtgtcaggggaagcccacaggagttaacagcagggttgcctcaacctagagaggaagcgacctggtgccctcggctctgtggcttccttcatctaacaacatcttccactctacaacaatgccagggaaggcggaggctggtacagtgcatcaagacacagctactcctgggtgacagaggttcagggccagctcactaagtaggcagaagtttttgacatatactttgagagataaagcaagattctgtacctcaaccttcagaatttcccctactgacctcttctcttcctcccacagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaatggaagcggagccaccaacttctccctgctgaagcaggccggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaagatacattgatgagtttggacaaaccacaactagaatgcagtgaaaaaaatgctttatttgtgaaatttgtgatgctattgctttatttgtaaccattataagctgcaataaacaagttattatagttccggagctatatagctcctatcattctatcataaccttagaataccagagaacatatcatctcatctaattatctcttactatatgtgaaaaaaatgaaggacatgggggaagtgtgacttgccccaaatcacatatttcatggtagagccaggtcttctgtttgtcatatcagtgttcttcctgccacaaccatcttgaagaatctatttctcagtaagaaaatatctttatggagagtagctggaaaacagttgagagatggaggggaggctgggggtgtggagaggggaaggggtaagtgatagattcgttgaaggggggagaaaaggccgtggggatgaagctagaaggcagaagggcttgcctgggcttggccatgaaggagcatgagttcactgagttccctaggcatgtctcatgctagctctcgat

SEQ ID NO: 21 - DNA, 3385 bp, synthetic construct
  source 1..3385: mol_type = other DNA
gtttaaaccctgcaggatttttgagttgatgcaagcctctctgtatggagagctggtctcctttatcctgtgggaaaagagaacaaaggagcatgggagtgttcaagggaaggagaaataaagggcagagaggcagcggtggtgtcaggggaagcccacaggagttaacagcagggttgcctcaacctagagaggaagcgacctggtgccctcggctctgtggcttccttcatctaacaacatcttccactctacaacaatgccagggaaggcggaggctggtacagtgcatcaagacacagctactcctgggtgacagaggttcagggccagctcactaagtaggcagaagtttttgacatatactttgagagataaagcaagattctgtacctcaaccttcagaatttcccctaactgacctcttctcttcctcccacagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaatggaagcggagccaccaacttctccctgctgaagcaggccggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttattatagttccggagctatatagctcctatcattctatcataaccttagaataccagagaacatatcatctcatctaattatctcttactatatgtgaaaaaaatgaaggacatgggggaagtgtgacttgccccaaatcacatatttcatggtagagccaggtcttctgtttgtcatatcagtgttcttcctgccacaaccatcttgaagaatctatttctcagtaagaaaatatctttatggagagtagctggaaaacagttgagagatggaggggaggctgggggtgtggagaggggaaggggtaagtgatagattcgttgaaggggggagaaaaggccgtggggatgaagctagaaggcagaagggcttgcctgggcttggccatgaaggagcatgagttcactgagttccctaggcatgtctcatgctagctctcgatgcttta

SEQ ID NO: 22 - DNA, 399 bp, synthetic construct
  source 1..399: mol_type = other DNA
atttttgagttgatgcaagcctctctgtatggagagctggtctcctttatcctgtgggaaaagagaacaaaggagcatgggagtgttcaagggaaggagaaataaagggcagagaggcagcggtggtgtcaggggaagcccacaggagttaacagcagggttgcctcaacctagagaggaagcgacctggtgccctcggctctgtggcttccttcatctaacaacatcttccactctacaacaatgccagggaaggcggaggctggtacagtgcatcaagacacagctactcctgggtgacagaggttcagggccagctcactaagtaggcagaagtttttgacatatactttgagagataaagcaagattctgtacctcaaccttcagaatttcccct

SEQ ID NO: 23 - DNA, 414 bp, synthetic construct
  source 1..414: mol_type = other DNA
attatagttccggagctatatagctcctatcattctatcataaccttagaataccagagaacatatcatctcatctaattatctcttactatatgtgaaaaaaatgaaggacatgggggaagtgtgacttgccccaaatcacatatttcatggtagagccaggtcttctgtttgtcatatcagtgttcttcctgccacaaccatcttgaagaatctatttctcagtaagaaaatatctttatggagagtagctggaaaacagttgagagatggaggggaggctgggggtgtggagaggggaaggggtaagtgatagattcgttgaaggggggagaaaaggccgtggggatgaagctagaaggcagaagggcttgcctgggcttggccatgaaggagcatgagttcactgagttc

SEQ ID NO: 24 - DNA, 26 bp, synthetic construct
  source 1..26: mol_type = other DNA
actgacctcttctcttcctcccacag

SEQ ID NO: 25 - DNA, 66 bp, synthetic construct
  source 1..66: mol_type = other DNA
ggaagcggagccaccaacttctccctgctgaagcaggccggcgacgtggaggagaaccccggcccc

SEQ ID NO: 26 - DNA, 21 bp, synthetic construct
  source 1..21: mol_type = other DNA
gctactcctgggtgacagagg

SEQ ID NO: 27 - DNA, 21 bp, synthetic construct
  source 1..21: mol_type = other DNA
tcatgtaggttgccgcacaga

SEQ ID NO: 28 - DNA, 20 bp, synthetic construct
  source 1..20: mol_type = other DNA
tggcttgccttggatttcag

SEQ ID NO: 29 - RNA, 20 nt, synthetic construct
  source 1..20: mol_type = other RNA
tggcttgccttggatttcag

SEQ ID NO: 30 - DNA, 11 bp, synthetic construct
  source 1..11: mol_type = other DNA
aaagccactga

SEQ ID NO: 31 - DNA, 560 bp, synthetic construct
  source 1..560: mol_type = other DNA
caatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaat